(19) United States
(12) Patent Application Publication
(10) Pub. No.: US 2025/0304648 A1
(43) Pub. Date: Oct. 2, 2025

(54) Variant Preproinsulin and Constructs for Insulin Expression and Treatment of Diabetes

(71) Applicant: Endsulin, Inc., Madison, WI (US)

(72) Inventor: Jennifer Gagne, Madison, WI (US)

(73) Assignee: Endsulin, Inc., Madison, WI (US)

(21) Appl. No.: 18/864,047

(22) PCT Filed: May 5, 2023
(86) PCT No.: PCT/US2023/066699

§ 371 Date: Nov. 8, 2024

Related U.S. Application Data
(60) Provisional application No. 63/339,910, filed on May 9, 2022.

(51) Int. Cl. C07K 14/62; A61K 38/00; C12N 15/86

(52) U.S. Cl. CPC C07K 14/62; C12N 15/86; A61K 38/00; C12N 2750/14143

(57) ABSTRACT

This invention relates to variant preproinsulin proteins and constructs encoding the same for the treatment of diabetes, including variant preproinsulin proteins having enzymatic cleavage sites that may be processed to form secreted, fully processed (or mature), active, wildtype insulin and mature wildtype C-peptide proteins.

### CROSS-REFERENCE TO RELATED APPLICATIONS

This application claims the benefit of priority of U.S. Provisional Application No. 63/339,910, filed May 9, 2022, which is incorporated by reference herein in its entirety for any purpose.

### REFERENCE TO SEQUENCE LISTING

The present application is filed with a sequence listing which has been submitted electronically in XML format. Said XML copy, created on Apr. 17, 2023, is named “01313-0001-00PCT_ST26.xml” and is 286,000 bytes in size. The information in the electronic format of the sequence listing is incorporated herein by reference in its entirety.

### FIELD

This disclosure relates to variant preproinsulin proteins and constructs encoding the same for the treatment of diabetes using cellular or gene therapies, such as hepatocyte-directed gene therapies, and includes variant preproinsulin proteins having enzymatic cleavage sites that may be processed to form secreted, fully processed (or mature), active, wildtype insulin and wildtype C-peptide proteins.

### BACKGROUND

Insulin is a protein normally produced in and secreted by the beta cells of the islets of Langerhans in the pancreas. The glucose responsive release of insulin from beta cells is a complex pathway involving gene expression, posttranslational modification, and secretion. Mature insulin is composed of two polypeptide chains, an A chain (21 amino acids) and a B chain (30 amino acids), held together by disulfide bonds. The precursor insulin protein product is preproinsulin, a single polypeptide chain that in addition to the B and A chains has an N-terminal signal sequence and an intervening sequence, named the C-peptide (31 amino acids), which is located between the B and A chains and connected on either side by two small two amino acid junctions. The sequence between the B chain and C-peptide is referred to as the “B/C junction,” while that between the C-peptide and A chain is referred to as the “C/A junction.” Cleavage of the signal peptide (24 amino acids) of preproinsulin yields proinsulin, which retains the C-peptide between the A and B chains.

Proinsulin is then folded into a complex three-dimensional structure with three disulfide bonds. Proper folding and disulfide bond formation is critical for insulin function and the accumulation of excess misfolded proinsulin has been shown to cause problems such as decreased insulin production, hyperglycemia, ER stress and even can cause forms of diabetes such as Mutant Ins-gene Induced Diabetes of Youth (MIDY). Liu, et al. (2010); Fonseca, et al. (2011). Following initial maturation and folding proinsulin is transported through the trans-Golgi, and packaged into secretory granules along with the endoproteases PC1/3 and PC2 that are unique to beta cells and required for processing of proinsulin to mature insulin. PC1/3 and PC2 recognize and cleave after the pairs of dibasic residues that comprise the B/C and C/A junctions of wildtype insulins. Following cleavage, the dibasic amino acids at the ends of the B-chain and C-peptide are removed by carboxypeptidases generating mature insulin and mature C-peptide. Mature (or fully processed) insulin and C-peptide, which are stored in secretory granules, are released extracellularly in response to elevated blood glucose levels. The detailed mechanism of insulin release is not completely understood, but the process involves migration to and fusion of the secretory granules with the plasma membrane prior to release.

In normally functioning beta cells, insulin production and release are affected by the glycolytic flux. Glucokinase and glucose transporter 2 (GLUT-2) are two proteins that are believed to be involved in sensing changes in the glucose concentration in beta cells. A reduction in GLUT-2, which is involved in glucose transport, is correlated with decreased expression of insulin and loss of glucokinase activity.

Diabetes occurs when the body is not able to take up glucose into its cells for use as energy which results in an accumulation of glucose in the bloodstream. There are multiple causes of diabetes. For example, autoimmune destruction of pancreatic beta cells causes insulin-dependent diabetes mellitus or Type I diabetes. Here with the partial or complete loss of beta cells, little or no insulin is secreted by the pancreas. This inadequate insulin production causes reduced glucose uptake and elevated blood glucose levels. Both reduced glucose uptake and high blood glucose levels are associated with very serious health problems, including amputations and cardiovascular and kidney diseases. In fact, without proper treatment, diabetes can be fatal.

One conventional treatment for diabetes involves the periodic administration of injectable exogenous insulin. This method has extended the life expectancy of millions of people with the disease. However, blood glucose levels must be carefully monitored to ensure that the individual receives an appropriate amount of insulin. Too much insulin can cause blood glucose levels to drop to dangerously low levels. Too little insulin will result in elevated blood glucose levels. Even with careful monitoring of blood glucose levels, control of diet, and insulin injections, the health of the vast majority of individuals with diabetes is adversely impacted in some way.

One alternative to the conventional periodic administration of injectable exogenous insulin is the replacement of beta cell function by allowing insulin to be secreted by other cells in response to glucose levels in the microenvironment. For example, replacing beta cell function with pancreas transplantation has met with some success. However, the supply of donors is quite limited, and this treatment requires long term immunosuppression. Additionally, pancreas transplantation is very costly and too problematic to be made widely available to those in need of beta cell function. Alternative methods of beta cell replacement have been proposed, including replacing beta cell function with donor beta cells or other insulin-secreting, pancreas-derived cell lines. Lacy et al. (1986). Unfortunately, because the immune system recognizes heterologous cells as foreign, these cells would need to be protected from immunoactive cells (e.g., T-cells and macrophages mediating cytolytic processes). Again, one approach to this is long-term immunosuppression while another approach to protect these heterologous cells is physical immunoisolation; however, immunoisolation itself poses significant problems.

One promising alternative to these potential methods which does not require long-term immunosuppression is gene therapy. Gene therapy is the treatment of a genetic disease by the introduction of specific cell function-altering genetic material into a patient. In one embodiment, gene delivery involves using vectors, either viral or non-viral vectors. Most commonly, viral vector-based gene therapy is achieved by in vivo delivery of the therapeutic gene into the patient by vectors based on retroviruses, adenoviruses (Ads) or adeno-associated viruses (AAVs). In another embodiment, a therapeutic transgene can be delivered ex vivo, whereby cells of a patient are extracted and cultured outside of the body. Cells are then genetically modified by introduction of a therapeutic transgene and re-introduced back into the patient. There are four basic gene therapy approaches as follows: gene replacement, the delivery of a functional gene to replace a non-working gene; gene silencing, inactivation of a mutated gene that has become toxic to cells; gene addition, expression of a “foreign” or exogenous gene to impact cellular function; and gene editing, a permanent manipulation of a gene in a patient's genome.

For an insulin gene therapy to be successful, the expressed insulin protein must undergo appropriate posttranslational folding and processing. The prohormone convertases, PC1/3 and PC2, required for insulin maturation are only expressed in β cells and other cells with the regulated secretory pathway (e.g., pituitary cells and intestinal K cells). Thus, a wildtype preproinsulin coding sequence that is expressed in the liver, for example, will result in unprocessed proinsulin (which has biological activity that is approximately 100-fold less than mature insulin) because the specific enzymes necessary for proteolytic processing are absent in liver cells.

To overcome the challenge of expressing and processing preproinsulin in constitutive secretory pathway cells, like those in the liver, the endoprotease sites of the insulin coding sequence may be modified to make it cleavable by another protease such as furin, thereby allowing proinsulin to be processed to mature insulin in constitutive secretory pathways cells, like those in the liver. For example, the human proinsulin coding sequence may be modified at the two junctions that are proteolytically processed: at the junction between the B-chain and C-peptide (from KTRR (SEQ ID NO: 204) to RTKR (SEQ ID NO: 205)) and at the junction between the C-peptide and A-chain (from LQKR (SEQ ID NO: 206) to RQKR (SEQ ID NO: 207)). See Simonson, et al. (1996).

However, the known variant proinsulin constructs whose amino acid sequences are modified to include furin cleavage sites generate a combination of non-wildtype products, including unprocessed variant proinsulin, which can only be reduced with furin co-expression, along with mature variant human insulin, and/or variant C-peptide. Groskreutz, et al. (1994); Yanagita, et al. (1992); Riu et al. (2002). It is known that inappropriate in vivo processing of proinsulin can carry significant health and safety risks. Specifically, unprocessed proinsulin molecules have been found to induce the unfolded protein response and undergo degradation in the endoplasmic reticulum, leading to severe endoplasmic reticulum stress and potentially p cell death by apoptosis. Støy, et al. (2007). Additionally, misfolded proinsulin proteins are known to cause problems such as decreased insulin production, hyperglycemia, and even can cause forms of diabetes such as Mutant Ins-gene Induced Diabetes of Youth (MIDY.) Liu, et al. (2010); Fonseca, et al. (2011).

If the known variant proinsulin constructs were used to treat patients, the production and accumulation of these variant products (e.g., unprocessed variant proinsulin, mature variant human insulin, and/or variant C-peptide) would pose serious health risks. First, as described above, there is a potential risk for R cell death due to ER toxicity caused by the accumulation of unprocessed proinsulin. Second, the accumulated unprocessed and processed modified proteins could be recognized as foreign by the immune system leading to autoimmune attacks against the organism's own cells if they were engineered to express the modified insulin. Shirley, et al. (2020). Finally, it has been suggested that there is an increased cancer risk for patients treated with certain insulin analogs, although further studies are needed. Some of these insulin analogs have similar mutations to those found in the current furin-cleavable variant proinsulin designs.

There are currently two variant proinsulin constructs in the art. One design (e.g., “the 1992 Yanagita design”) generates a combination of mature wildtype human insulin, a truncated C-peptide, and unprocessed variant proinsulin. Yanagita, et al. (1992). The other recombinant proinsulin design in the art (e.g., “the 1994 Groskreutz design”) generates a combination of mature variant human insulin, a variant C-peptide, and unprocessed variant proinsulin. Groskreutz, et al. (1994). Because the variant proinsulin constructs in the art result in production of unprocessed variant proinsulin, mature variant human insulin and/or variant C-peptide, as described above, those treatment options are associated with risks of protein degradation, cell death, immunological reaction, toxicity, cancer, and other health concerns. Therefore, there exists a need in the field for modified proinsulin constructs that can be expressed in cells, other than beta cells, and may be completely processed into mature wildtype insulin and wildtype C-peptide so as to avoid the safety concerns associated with the current options.

Furthermore, the existing variant proinsulin constructs based on the 1992 Yanagita and 1994 Groskreutz designs each produce a variant C-peptide which is not detectable in serum samples using many of the currently available c-peptide detection methods which are used as an indirect means of monitoring serum insulin levels. Hence, there remains a need for modified proinsulin constructs that are not only capable of being expressed and processed in cells, other than beta cells, but that also produce wildtype C-peptide, which can be detected and monitored using commercially available detection kits. The existence of such a variant proinsulin is especially critical for patient monitoring if variant proinsulins are eventually used in a therapeutic.

Unlike the other constructs, the variant preproinsulin constructs disclosed herein may be expressed and processed in constitutive secretory pathway cells, like those in the liver and may result in complete processing into mature wildtype insulin and wildtype C-peptide. Such constructs alleviate many safety concerns associated with the prior constructs and provide for improved patient monitoring.

### SUMMARY

This disclosure provides nucleic acid molecules comprising:
a) an N-terminal signal sequence,
b) a wildtype B-chain or full-length variant thereof comprising at least one amino acid substitution,
c) a variant B/C junction comprising at least 4 amino acids selected from histidine, lysine, and arginine,
d) a wildtype C-peptide or full-length variant thereof comprising at least one amino acid substitution,
e) a variant C/A junction comprising at least 4 amino acids selected from histidine, lysine, and arginine, and
f) a wildtype A-chain or full-length variant thereof comprising at least one amino acid substitution,
   wherein the variant B/C junction and the variant C/A junction each comprise an enzymatic cleavage site for a target proteolytic enzyme to cleave immediately after the C-terminal amino acid of the junction.

In some embodiments, the enzymatic cleavage site is a subtilisin-like proprotein convertase cleavage site. In some embodiments, the enzymatic cleavage site is a furin cleavage site.

In some embodiments, the preproinsulin polypeptide is capable of being processed into a mature wildtype insulin protein or full-length variant thereof comprising at least one amino acid substitution, and a mature wildtype C-peptide or full-length variant thereof comprising at least one amino acid substitution. In some embodiments, the preproinsulin polypeptide is capable of being processed by furin and a carboxypeptidase into a mature wildtype insulin protein or full-length variant thereof comprising at least one amino acid substitution, and a mature wildtype C-peptide or full-length variant thereof comprising at least one amino acid substitution.

In some embodiments, the mature wildtype insulin protein is a mature wildtype human insulin protein, mature wildtype canine insulin protein, or mature wildtype feline insulin protein; and wherein the mature wildtype C-peptide is a mature wildtype human C-peptide, mature wildtype canine C-peptide, or mature wildtype feline C-peptide. In some embodiments, the mature wildtype insulin protein is a mature wildtype human insulin protein; and wherein the mature wildtype C-peptide is a mature wildtype human C-peptide.

In some embodiments, the enzymatic cleavage site comprises an amino acid sequence of RX₁X₂R (SEQ ID NO: 45), wherein X₁ is histidine, lysine, or arginine and X₂ is lysine or arginine. In some embodiments, each enzymatic cleavage site comprises an amino acid sequence selected from RHKR (SEQ ID NO: 52), RHRR (SEQ ID NO: 53), RKKR (SEQ ID NO: 54), RKRR (SEQ ID NO: 55), RRKR (SEQ ID NO: 56), and RRRR (SEQ ID NO: 57). In some embodiments, each enzymatic cleavage site comprises an amino acid sequence of RRKR (SEQ ID NO: 56).

In some embodiments, the variant B/C junction and the variant C/A junction each comprise 4 to 10 amino acids selected from histidine, lysine, and arginine. In some embodiments, the variant B/C junction and the variant C/A junction each comprise 4 to 6 amino acids selected from histidine, lysine, and arginine. In some embodiments, the variant B/C junction and the variant C/A junction each comprise 4 amino acids selected from histidine, lysine, and arginine.

In some embodiments, the variant B/C junction comprises an amino acid sequence of XₙRX₁X₂R (SEQ ID NO: 64), wherein Xₙ is 0, 1, 2, 3, 4, 5, or 6 amino acids each chosen from histidine, lysine, or arginine, X₁ is histidine, lysine, or arginine, and X₂ is lysine or arginine. In some embodiments, the variant B/C junction comprises an amino acid sequence of SEQ ID NO: 52, SEQ ID NO: 53, SEQ ID NO: 54, SEQ ID NO: 55, SEQ ID NO: 56, SEQ ID NO: 57, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, or SEQ ID NO: 199. In some embodiments, the variant B/C junction comprises an amino acid sequence of SEQ ID NO: 52, SEQ ID NO: 53, SEQ ID NO: 54, SEQ ID NO: 55, SEQ ID NO: 56, SEQ ID NO: 57, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, or SEQ ID NO: 136. In some embodiments, the variant B/C junction comprises an amino acid sequence of RRKR (SEQ ID NO: 56).

In some embodiments, the variant C/A junction comprises an amino acid sequence of XₙRX₁X₂R (SEQ ID NO: 64), wherein Xₙ is 0, 1, 2, 3, 4, 5, or 6 amino acids each chosen from histidine, lysine, or arginine, X₁ is histidine, lysine, or arginine, and X₂ is lysine or arginine. In some embodiments, the variant C/A junction comprises an amino acid sequence of SEQ ID NO: 52, SEQ ID NO: 53, SEQ ID NO: 54, SEQ ID NO: 55, SEQ ID NO: 56, SEQ ID NO: 57, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, or SEQ ID NO: 199. In some embodiments, the variant C/A junction comprises an amino acid sequence of SEQ ID NO: 52, SEQ ID NO: 53, SEQ ID NO: 54, SEQ ID NO: 55, SEQ ID NO: 56, SEQ ID NO: 57, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, or SEQ ID NO: 136. In some embodiments, the variant C/A junction comprises an amino acid sequence of RRKR (SEQ ID NO: 56).

In some embodiments, the wildtype B-chain is a wildtype human B-chain, a wildtype canine B-chain, or a wildtype feline B-chain. In some embodiments, the wildtype B-chain comprises an amino acid sequence of SEQ ID NO: 59 or SEQ ID NO: 66. In some embodiments, the wildtype B-chain is a wildtype human B-chain. In some embodiments, the wildtype B-chain or full-length variant thereof comprises an amino acid sequence of SEQ ID NO: 59 or SEQ ID NO: 203.

In some embodiments, the wildtype C-peptide is a wildtype human C-peptide, a wildtype canine C-peptide, or a wildtype feline C-peptide. In some embodiments, the wildtype C-peptide comprises an amino acid sequence of SEQ ID NO: 60, SEQ ID NO: 67, or SEQ ID NO: 70. In some embodiments, the wildtype C-peptide is a wildtype human C-peptide. In some embodiments, the wildtype C-peptide comprises an amino acid sequence of SEQ ID NO: 60.

In some embodiments, the wildtype A-chain is a wildtype human A-chain, a wildtype canine A-chain, or a wildtype feline A-chain. In some embodiments, the wildtype A-chain comprises an amino acid sequence of SEQ ID NO: 61, SEQ ID NO: 68, or SEQ ID NO: 71. In some embodiments, the wildtype A-chain is a wildtype human A-chain. In some embodiments, the wildtype A-chain comprises an amino acid sequence of SEQ ID NO: 61.

In some embodiments, the preproinsulin polypeptide is capable of being processed into a proinsulin polypeptide comprising an amino acid sequence of SEQ ID NO: 2, SEQ ID NO: 72, or SEQ ID NO: 73. In some embodiments, the preproinsulin polypeptide is capable of being processed into a proinsulin polypeptide comprising an amino acid sequence of SEQ ID NO: 2. In some embodiments, the preproinsulin polypeptide is capable of being processed into a proinsulin polypeptide comprising an amino acid sequence of SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 74, or SEQ ID NO: 75. In some embodiments, the preproinsulin polypeptide is capable of being processed into a proinsulin polypeptide comprising an amino acid sequence of SEQ ID NO: 175.

This disclosure provides an nucleic acid molecule comprising a nucleic acid sequence encoding a proinsulin polypeptide comprising an amino acid sequence of SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 74, or SEQ ID NO: 75.

This disclosure provides a nucleic acid molecule comprising a nucleic acid sequence encoding a proinsulin polypeptide comprising an amino acid sequence of SEQ ID NO: 175.

In some embodiments, the N-terminal signal sequence comprises a wildtype N-terminal signal sequence. In some embodiments, the N-terminal signal sequence comprises a wildtype human N-terminal signal sequence, a wildtype canine N-terminal signal sequence, or a wildtype feline N-terminal signal sequence. In some embodiments, the N-terminal signal sequence comprises an amino acid sequence of SEQ ID NO: 43, SEQ ID NO: 65, or SEQ ID NO: 69. In some embodiments, the N-terminal signal sequence comprises a wildtype human N-terminal signal sequence. In some embodiments, the N-terminal signal sequence comprises an amino acid sequence of SEQ ID NO: 43. In some embodiments, the N-terminal signal sequence comprises a variant N-terminal signal sequence.

In some embodiments, the variant preproinsulin polypeptide comprises an amino acid sequence of SEQ ID NO: 1, SEQ ID NO: 48, or SEQ ID NO: 51. In some embodiments, the variant preproinsulin polypeptide comprises an amino acid sequence of SEQ ID NO: 1. In some embodiments, the variant preproinsulin polypeptide comprises an amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 47, or SEQ ID NO: 50. In some embodiments, the variant preproinsulin polypeptide comprises an amino acid sequence of SEQ ID NO: 31.

This disclosure provides a nucleic acid molecule comprising a nucleic acid sequence encoding a variant preproinsulin polypeptide comprising an amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 47, or SEQ ID NO: 50.

This disclosure provides a nucleic acid molecule encoding a variant preproinsulin polypeptide comprising an amino acid sequence of SEQ ID NO: 31.

In some embodiments, the nucleic acid molecule comprises a nucleic acid sequence of SEQ ID NO: 77.

This disclosure provides variant preproinsulin polypeptides comprising:
a) an N-terminal signal sequence,
b) a wildtype B-chain or full-length variant thereof comprising at least one amino acid substitution,
c) a variant B/C junction comprising at least 4 amino acids selected from histidine, lysine, and arginine,
d) a wildtype C-peptide or full-length variant thereof comprising at least one amino acid substitution,
e) a variant C/A junction comprising at least 4 amino acids selected from histidine, lysine, and arginine, and
f) a wildtype A-chain or full-length variant thereof comprising at least one amino acid substitution,
   wherein the variant B/C junction and the variant C/A junction each comprise an enzymatic cleavage site for a target proteolytic enzyme to cleave immediately after the C-terminal amino acid of the junction.

In some embodiments, the enzymatic cleavage site is a subtilisin-like proprotein convertase cleavage site. In some embodiments, the enzymatic cleavage site is a furin cleavage site.

In some embodiments, the preproinsulin polypeptide is capable of being processed into a mature wildtype insulin protein or full-length variant thereof comprising at least one amino acid substitution, and a mature wildtype C-peptide or full-length variant thereof comprising at least one amino acid substitution. In some embodiments, the preproinsulin polypeptide is capable of being processed by furin and a carboxypeptidase into a mature wildtype insulin protein or full-length variant thereof comprising at least one amino acid substitution, and a mature wildtype C-peptide or full-length variant thereof comprising at least one amino acid substitution.

In some embodiments, the mature wildtype insulin protein is a mature wildtype human insulin protein, mature wildtype canine insulin protein, or mature wildtype feline insulin protein; and wherein the mature wildtype C-peptide is a mature wildtype human C-peptide, mature wildtype canine C-peptide, or mature wildtype feline C-peptide. In some embodiments, the mature wildtype insulin protein is a mature wildtype human insulin protein; and wherein the mature wildtype C-peptide is a mature wildtype human C-peptide.

In some embodiments, the enzymatic cleavage site comprises an amino acid sequence of RX₁X₂R (SEQ ID NO: 45), wherein X₁ is histidine, lysine, or arginine and X₂ is lysine or arginine. In some embodiments, each enzymatic cleavage site comprises an amino acid sequence selected from RHKR (SEQ ID NO: 52), RHRR (SEQ ID NO: 53), RKKR (SEQ ID NO: 54), RKRR (SEQ ID NO: 55), RRKR (SEQ ID NO: 56), and RRRR (SEQ ID NO: 57). In some embodiments, each enzymatic cleavage site comprises an amino acid sequence of RRKR (SEQ ID NO: 56).

In some embodiments, the variant B/C junction and the variant C/A junction each comprise 4 to 10 amino acids selected from histidine, lysine, and arginine. In some embodiments, the variant B/C junction and the variant C/A junction each comprise 4 to 6 amino acids selected from histidine, lysine, and arginine. In some embodiments, the variant B/C junction and the variant C/A junction each comprise 4 amino acids selected from histidine, lysine, and arginine.

In some embodiments, the variant B/C junction comprises an amino acid sequence of XₙRX₁X₂R (SEQ ID NO: 64), wherein Xₙ is 0, 1, 2, 3, 4, 5, or 6 amino acids each chosen from histidine, lysine, or arginine, X₁ is histidine, lysine, or arginine, and X₂ is lysine or arginine. In some embodiments, the variant B/C junction comprises an amino acid sequence of SEQ ID NO: 52, SEQ ID NO: 53, SEQ ID NO: 54, SEQ ID NO: 55, SEQ ID NO: 56, SEQ ID NO: 57, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, or SEQ ID NO: 136. In some embodiments, the variant B/C junction comprises an amino acid sequence of RRKR (SEQ ID NO: 56).

In some embodiments, the variant C/A junction comprises an amino acid sequence of XₙRX₁X₂R (SEQ ID NO: 64), wherein Xₙ is 0, 1, 2, 3, 4, 5, or 6 amino acids each chosen from histidine, lysine, or arginine, X₁ is histidine, lysine, or arginine, and X₂ is lysine or arginine. In some embodiments, the variant C/A junction comprises an amino acid sequence of SEQ ID NO: 52, SEQ ID NO: 53, SEQ ID NO: 54, SEQ ID NO: 55, SEQ ID NO: 56, SEQ ID NO: 57, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, or SEQ ID NO: 136. In some embodiments, the variant C/A junction comprises an amino acid sequence of RRKR (SEQ ID NO: 56).

In some embodiments, the wildtype B-chain is a wildtype human B-chain, a wildtype canine B-chain, or a wildtype feline B-chain. In some embodiments, the wildtype B-chain comprises an amino acid sequence of SEQ ID NO: 59 or SEQ ID NO: 66. In some embodiments, the wildtype B-chain is a wildtype human B-chain. In some embodiments, the wildtype B-chain or full-length variant thereof comprises an amino acid sequence of SEQ ID NO: 59 or SEQ ID NO: 203.

In some embodiments, the wildtype C-peptide is a wildtype human C-peptide, a wildtype canine C-peptide, or a wildtype feline C-peptide. In some embodiments, the wildtype C-peptide comprises an amino acid sequence of SEQ ID NO: 60, SEQ ID NO: 67, or SEQ ID NO: 70. In some embodiments, the wildtype C-peptide is a wildtype human C-peptide. In some embodiments, the wildtype C-peptide comprises an amino acid sequence of SEQ ID NO: 60.

In some embodiments, the wildtype A-chain is a wildtype human A-chain, a wildtype canine A-chain, or a wildtype feline A-chain. In some embodiments, the wildtype A-chain comprises an amino acid sequence of SEQ ID NO: 61, SEQ ID NO: 68, or SEQ ID NO: 71. In some embodiments, the wildtype A-chain is a wildtype human A-chain. In some embodiments, the wildtype A-chain comprises an amino acid sequence of SEQ ID NO: 61.

In some embodiments, the preproinsulin polypeptide is capable of being processed into a proinsulin polypeptide comprising an amino acid sequence of SEQ ID NO: 2, SEQ ID N: 72, or SEQ ID NO: 73. In some embodiments, the preproinsulin polypeptide is capable of being processed into a proinsulin polypeptide comprising an amino acid sequence of SEQ ID NO: 2. In some embodiments, the preproinsulin polypeptide is capable of being processed into a proinsulin polypeptide comprising an amino acid sequence of SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 74, or SEQ ID NO: 75. In some embodiments, the preproinsulin polypeptide is capable of being processed into a proinsulin polypeptide comprising an amino acid sequence of SEQ ID NO: 175.

In some embodiments, the N-terminal signal sequence comprises a wildtype N-terminal signal sequence. In some embodiments, the N-terminal signal sequence comprises a wildtype human N-terminal signal sequence, a wildtype canine N-terminal signal sequence, or a wildtype feline N-terminal signal sequence. In some embodiments, the N-terminal signal sequence comprises an amino acid sequence of SEQ ID NO: 43, SEQ ID NO: 65, or SEQ ID NO: 69. In some embodiments, the N-terminal signal sequence comprises a wildtype human N-terminal signal sequence. In some embodiments, the N-terminal signal sequence comprises an amino acid sequence of SEQ ID NO: 43. In some embodiments, the N-terminal signal sequence comprises a variant N-terminal signal sequence.

In some embodiments, the variant preproinsulin polypeptide comprises an amino acid sequence of SEQ ID NO: 1, SEQ ID NO: 48, or SEQ ID NO: 51. In some embodiments, the variant preproinsulin polypeptide comprises an amino acid sequence of SEQ ID NO: 1. In some embodiments, the variant preproinsulin polypeptide comprises an amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 47, or SEQ ID NO: 50. In some embodiments, the variant preproinsulin polypeptide comprises an amino acid sequence of SEQ ID NO: 31.

This disclosure provides a nucleic acid molecule comprising a nucleic acid sequence encoding the variant preproinsulin polypeptide as described herein.

In some embodiments, the nucleic acid molecule further comprises a promoter operatively linked to the nucleic acid sequence encoding the variant preproinsulin polypeptide. In some embodiments, the promoter is a constitutive promoter. In some embodiments, the promoter is a regulated promoter. In some embodiments, the promoter is an albumin promoter. In some embodiments, the nucleic acid molecule further comprises at least one GIRE element.

This disclosure provides a vector comprising the nucleic acid as described herein. In some embodiments, the vector comprises a nucleic acid comprising a nucleic acid sequence of SEQ ID NO: 76 or SEQ ID NO: 77. In some embodiments, the vector is a virus vector. In some embodiments, the vector is an adeno-associated virus (AAV) vector, an adenovirus vector, a retrovirus vector, a herpesvirus vector, or a pox virus vector. In some embodiments, the vector is an adeno-associated virus (AAV) vector. In some embodiments, the vector is a self-complementary adeno-associated virus (scAAV) vector. In some embodiments, the vector is an adeno-associated virus (AAV) vector having a capsid serotype selected from the group consisting of AAV1, AAV2, AAV3, AAV4, AAV5, AAV6, AAV7, AAV8, AAV9, AAV10, AAV11, AAV12, AAV13, and any variant thereof. In some embodiments, the AAV vector has a capsid serotype of AAV8. In some embodiments, the vector is a synthetic mRNA. In some embodiments, the vector is a self-replicating RNA.

This disclosure provides a cultured host cell comprising a nucleic acid molecule as described herein, a variant preproinsulin polypeptide as described herein, or a vector as described herein.

This disclosure provides a pharmaceutical composition comprising a nucleic acid molecule as described herein, a variant preproinsulin polypeptide as described herein, a vector as described herein, or a cultured host cell as described herein and a pharmaceutically acceptable carrier.

This disclosure provides a method of treating a subject with diabetes comprising administering to the subject a nucleic acid molecule as described herein, a variant preproinsulin polypeptide as described herein, a vector as described herein, a cultured host cell as described herein, or a pharmaceutical composition as described herein. In some embodiments, the diabetes in Type 1 diabetes.

In some embodiments, the nucleic acid, the variant preproinsulin polypeptide, the vector, the cultured host cell, and/or the pharmaceutical composition is administered to the subject via intravenous injection, arterial injection, intramuscular injection, intradermal injection, intraperitoneal injection, and/or subcutaneous injection.

In some embodiments, the vector is administered to the subject at a dose of about 1×10¹⁰ vector genomes per kilogram, about 1×10¹¹ vector genomes per kilogram, about 1×10¹² vector genomes per kilogram, about 1×10¹³ vector genomes per kilogram, about 1×10¹⁰ to about 1×10¹³ vector genomes per kilogram, about 1×10¹⁰ to about 1×10¹ vector genomes per kilogram, about 1×10¹¹ to about 1×10¹² vector genomes per kilogram, or about 1×10¹⁰ to about 1×10¹¹ vector genomes per kilogram.

In some embodiments, the subject is a human, a dog, or a cat. In some embodiments, the subject is a human subject.

This disclosure provides a method of producing insulin in a cell, the method comprising transducing, transfecting, or transforming the cell with a nucleic acid molecule as described herein or a vector as described herein. In some embodiments, the cell is exposed to the nucleic acid or the vector ex vivo. In some embodiments, the cell is exposed to the nucleic acid or the vector in vivo. In some embodiments, the cell is a human cell, a canine cell, or a feline cell. In some embodiments, the cell is a liver cell or a muscle cell.

### BRIEF DESCRIPTION OF THE DRAWINGS

FIG. 1 provides an alignment of the human wildtype preproinsulin sequence (SEQ ID NO: 39) as compared to the 1994 Groskreutz-Human and 1992 Yanagita-Human sequences (SEQ ID NOs: 40 and 41, respectively). For the preproinsulin and proinsulin forms there are three amino acid changes between wildtype human preproinsulin and 1994 Groskreutz-Human and four amino acid changes between wildtype human preproinsulin and 1992 Yanagita-Human.

FIG. 2 shows an alignment of the human wildtype preproinsulin (SEQ ID NO: 39), 1994 Groskreutz-Human preproinsulin (SEQ ID NO: 40), and 1992 Yanagita-Human preproinsulin (SEQ ID NO: 41) sequences and their predicted processing sites. The cleavage sites and the locations of the amino acids removed by carboxypeptidase are similar for wildtype Human preproinsulin and 1994 Groskreutz-Human. For 1992 Yanagita-Human, one of the cleavage sites is shifted by two amino acids compared to wildtype human preproinsulin and four more amino acids end up being removed using carboxypeptidase.

FIG. 3 shows an alignment of the sequences of the final mature insulin B-chain and A-chain products generated by the cleavage of the different constructs. The mature insulin generated by 1992 Yanagita-Human (SEQ ID NO: 41) is identical to mature wildtype human (SEQ ID NO: 39) insulin. The mature insulin generated by 1994 Groskreutz-Human (SEQ ID NO: 40) has a single charge conserved (K→R) amino acid change at the penultimate amino acid of the B-chain.

FIG. 4 shows an alignment of the sequences of the final C-peptide products. The mature C-peptide generated by processing 1992 Yanagita-Human (SEQ ID NO: 41) is four amino acids smaller than the mature C-peptide generated by processing wildtype human insulin (SEQ ID NO: 39) but otherwise has the identical amino acid sequence. The mature C-peptide generated by 1994 Groskreutz-Human (SEQ ID NO: 40) has a single non-conserved (L→R) amino acid change at the penultimate amino acid when compared to mature wildtype human C-peptide.

FIG. 5 shows an alignment of the ENDSULIN101 (SEQ ID NO: 31) preproinsulin sequence and the processing sites thereof in comparison to that of wildtype Human (SEQ ID NO: 39), 1994 Groskreutz-Human (SEQ ID NO: 40) and 1992 Yanagita-Human preproinsulin (SEQ ID NO: 41). The preproinsulin and proinsulin forms of ENDSULIN101-Human have four additional amino acids than wildtype Human, 1994 Groskreutz-Human and 1992 Yanagita-Human preproinsulin. The final mature insulin and mature C-peptide generated from ENDSULIN101 are identical to the mature wildtype human insulin and mature C-peptide.

FIG. 6 is a schematic showing the sequence of insulin processing, beginning with the production of the preproinsulin protein. The figure is adapted from Yang, et al. (2010).

FIGS. 7A-B are schematics showing insulin processing intermediates, beginning with proinsulin. FIG. 7A shows insulin processing intermediates, including des 64,65 and des 31,32, as well as fully processed insulin and C-peptide. FIG. 7B shows wildtype, 1994 Groskreutz, and 1992 Yanagita proinsulin processing. As labeled the percentages reflect the amounts of unprocessed proinsulin, partially processed proinsulin, and mutated C-peptide or mutated insulin observed for 1994 Groskreutz or unprocessed proinsulin, partially processed proinsulin, insulin, and truncated C-peptide observed for 1992 Yanagita reported in the literature and based on ELISA results from Example 4.

FIG. 8 is a chart showing the concentration of mature human insulin expressed as detected by the Mercodia Mature Human Insulin ELISA according to Example 4B. Media from cells transduced with 5 different vectors (x-axis) was quantified for the presence of mature human insulin. The y-axis represents the concentration in ng/mL.

FIG. 9 is a chart showing the concentration of human C-peptide expressed as detected by the Mercodia Human C-peptide ELISA according to Example 4C. Media from cells transduced with 5 different vectors (x-axis) was quantified for the presence of human C-peptide. The y-axis represents the concentration in ng/mL.

FIG. 10 is a chart showing the concentration of human C-peptide expressed as detected by the Crystal Chem Human C-peptide ELISA according to Example 4D. Media from cells transduced with 5 different vectors (x-axis) was quantified for the presence of human C-peptide. The y-axis represents the concentration in ng/mL.

FIG. 11 is a chart showing the concentration of human proinsulin expressed as detected by the Mercodia Human Proinsulin ELISA according to Example 4E. Media from cells transduced with 5 different vectors (x-axis) was quantified for the presence of proinsulin. The y-axis represents the concentration in ng/mL.

FIG. 12 is a chart showing the concentration of all insulin products expressed as detected by the Mercodia Iso-Insulin ELISA according to Example 4F. Media from cells transduced with 5 different vectors (x-axis) was quantified for the presence of any insulin species regardless of processing or species. The y-axis represents the concentration in ng/mL.

FIG. 13 is a chart showing the concentration of rat insulin expressed as detected by the Mercodia Rat Insulin ELISA according to Example 4G. This ELISA recognizes insulins from other species with varying sensitivity. Media from cells transduced with 5 different vectors (x-axis) was quantified for the presence of rat insulin. The y-axis represents the concentration in ng/mL.

FIG. 14 is a chart showing the concentration of rat C-peptide expressed as detected by the Mercodia Rat C-peptide ELISA according to Example 4H. Media from cells transduced with 5 different vectors (x-axis) was quantified for the presence of rat C-peptide. The y-axis represents the concentration in ng/mL.

### DESCRIPTION OF CERTAIN SEQUENCES

Table 1 provides a listing of certain sequences referenced herein.

TABLE 1
Description of Certain Sequences

| | | SEQ ID |
|---|---|---|
| Description | Sequence | NO |
| Exemplary Variant | MALWMRLLPLLALLALWGPDPAAAFVNQHLCGSHL | 1 |
| Human | VEALYLVCGERGFFYTPKTXₙRX₁X₂REAEDLQVGQ | |
| Preproinsulin | VELGGGPGAGSLQPLALEGSLQXₙRX₁X₂RGIVEQC | |
| Consensus based on | CTSICSLYQLENYCN | |
| ENDSULIN101 | wherein Xₙ is 0, 1, 2, 3, 4, 5, or 6 amino acids each | |
| Preproinsulin | selected from H, K, and R; wherein X₁ is H, K, or R; | |
| | and wherein X₂ is R or K. | |
| Exemplary Variant | FVNQHLCGSHLVEALYLVCGERGFFYTPKTXₙRX₁X₂ | 2 |
| Human Proinsulin; | REAEDLQVGQVELGGGPGAGSLQPLALEGSLQXₙR | |
| Consensus based on | X₁X₂RGIVEQCCTSICSLYQLENYCN | |
| ENDSULIN101 | wherein Xₙ is 0, 1, 2, 3, 4, 5, or 6 amino acids each | |
| Proinsulin | selected from H, K, and R; wherein X₁ is H, K, or R; | |
| | and wherein X₂ is R or K. | |
| Exemplary | MALWMRLLPLLALLALWGPDPAAAFVNQHLCGSHL | 3 |
| preproinsulin 1 | VEALYLVCGERGFFYTPKTRHKREAEDLQVGQVEL | |
| | GGGPGAGSLQPLALEGSLQRHKRGIVEQCCTSICS | |
| | LYQLENYCN | |
| Exemplary | MALWMRLLPLLALLALWGPDPAAAFVNQHLCGSHL | 4 |
| preproinsulin 2 | VEALYLVCGERGFFYTPKTRHKREAEDLQVGQVEL | |
| | GGGPGAGSLQPLALEGSLQRHRRGIVEQCCTSICS | |
| | LYQLENYCN | |
| Exemplary | MALWMRLLPLLALLALWGPDPAAAFVNQHLCGSHL | 5 |
| preproinsulin 3 | VEALYLVCGERGFFYTPKTRHKREAEDLQVGQVEL | |
| | GGGPGAGSLQPLALEGSLQRKKRGIVEQCCTSICS | |
| | LYQLENYCN | |
| Exemplary | MALWMRLLPLLALLALWGPDPAAAFVNQHLCGSHL | 6 |
| preproinsulin 4 | VEALYLVCGERGFFYTPKTRHKREAEDLQVGQVEL | |
| | GGGPGAGSLQPLALEGSLQRKRRGIVEQCCTSICS | |
| | LYQLENYCN | |
| Exemplary | MALWMRLLPLLALLALWGPDPAAAFVNQHLCGSHL | 7 |
| preproinsulin 5 | VEALYLVCGERGFFYTPKTRHKREAEDLQVGQVEL | |
| | GGGPGAGSLQPLALEGSLQRRKRGIVEQCCTSICS | |
| | LYQLENYCN | |
| Exemplary | MALWMRLLPLLALLALWGPDPAAAFVNQHLCGSHL | 8 |
| preproinsulin 6 | VEALYLVCGERGFFYTPKTRHKREAEDLQVGQVEL | |
| | GGGPGAGSLQPLALEGSLQRRRRGIVEQCCTSICS | |
| | LYQLENYCN | |
| Exemplary | MALWMRLLPLLALLALWGPDPAAAFVNQHLCGSHL | 9 |
| preproinsulin 7 | VEALYLVCGERGFFYTPKTRHRREAEDLQVGQVEL | |
| | GGGPGAGSLQPLALEGSLQRHKRGIVEQCCTSICS | |
| | LYQLENYCN | |
| Exemplary | MALWMRLLPLLALLALWGPDPAAAFVNQHLCGSHL | 10 |
| preproinsulin 8 | VEALYLVCGERGFFYTPKTRHRREAEDLQVGQVEL | |
| | GGGPGAGSLQPLALEGSLQRHRRGIVEQCCTSICS | |
| | LYQLENYCN | |
| Exemplary | MALWMRLLPLLALLALWGPDPAAAFVNQHLCGSHL | 11 |
| preproinsulin 9 | VEALYLVCGERGFFYTPKTRHRREAEDLQVGQVEL | |
| | GGGPGAGSLQPLALEGSLQRKKRGIVEQCCTSICS | |
| | LYQLENYCN | |
| Exemplary | MALWMRLLPLLALLALWGPDPAAAFVNQHLCGSHL | 12 |
| preproinsulin 10 | VEALYLVCGERGFFYTPKTRHRREAEDLQVGQVEL | |
| | GGGPGAGSLQPLALEGSLQRKRRGIVEQCCTSICS | |
| | LYQLENYCN | |
| Exemplary | MALWMRLLPLLALLALWGPDPAAAFVNQHLCGSHL | 13 |
| preproinsulin 11 | VEALYLVCGERGFFYTPKTRHRREAEDLQVGQVEL | |
| | GGGPGAGSLQPLALEGSLQRRKRGIVEQCCTSICS | |
| | LYQLENYCN | |
| Exemplary | MALWMRLLPLLALLALWGPDPAAAFVNQHLCGSHL | 14 |
| preproinsulin 12 | VEALYLVCGERGFFYTPKTRHRREAEDLQVGQVEL | |
| | GGGPGAGSLQPLALEGSLQRRRRGIVEQCCTSICS | |
| | LYQLENYCN | |
| Exemplary | MALWMRLLPLLALLALWGPDPAAAFVNQHLCGSHL | 15 |
| preproinsulin 13 | VEALYLVCGERGFFYTPKTRKKREAEDLQVGQVEL | |
| | GGGPGAGSLQPLALEGSLQRHKRGIVEQCCTSICS | |
| | LYQLENYCN | |
| Exemplary | MALWMRLLPLLALLALWGPDPAAAFVNQHLCGSHL | 16 |
| preproinsulin 14 | VEALYLVCGERGFFYTPKTRKKREAEDLQVGQVEL | |
| | GGGPGAGSLQPLALEGSLQRHRRGIVEQCCTSICS | |
| | LYQLENYCN | |
| Exemplary | MALWMRLLPLLALLALWGPDPAAAFVNQHLCGSHL | 17 |
| preproinsulin 15 | VEALYLVCGERGFFYTPKTRKKREAEDLQVGQVEL | |
| | GGGPGAGSLQPLALEGSLQRKKRGIVEQCCTSICS | |
| | LYQLENYCN | |
| Exemplary | MALWMRLLPLLALLALWGPDPAAAFVNQHLCGSHL | 18 |
| preproinsulin 16 | VEALYLVCGERGFFYTPKTRKKREAEDLQVGQVEL | |
| | GGGPGAGSLQPLALEGSLQRKRRGIVEQCCTSICS | |
| | LYQLENYCN | |
| Exemplary | MALWMRLLPLLALLALWGPDPAAAFVNQHLCGSHL | 19 |
| preproinsulin 17 | VEALYLVCGERGFFYTPKTRKKREAEDLQVGQVEL | |
| | GGGPGAGSLQPLALEGSLQRRKRGIVEQCCTSICS | |
| | LYQLENYCN | |
| Exemplary | MALWMRLLPLLALLALWGPDPAAAFVNQHLCGSHL | 20 |
| preproinsulin 18 | VEALYLVCGERGFFYTPKTRKKREAEDLQVGQVEL | |
| | GGGPGAGSLQPLALEGSLQRRRRGIVEQCCTSICS | |
| | LYQLENYCN | |
| Exemplary | MALWMRLLPLLALLALWGPDPAAAFVNQHLCGSHL | 21 |
| preproinsulin 19 | VEALYLVCGERGFFYTPKTRKRREAEDLQVGQVEL | |
| | GGGPGAGSLQPLALEGSLQRHKRGIVEQCCTSICS | |
| | LYQLENYCN | |
| Exemplary | MALWMRLLPLLALLALWGPDPAAAFVNQHLCGSHL | 22 |
| preproinsulin 20 | VEALYLVCGERGFFYTPKTRKRREAEDLQVGQVEL | |
| | GGGPGAGSLQPLALEGSLQRHRRGIVEQCCTSICS | |
| | LYQLENYCN | |
| Exemplary | MALWMRLLPLLALLALWGPDPAAAFVNQHLCGSHL | 23 |
| preproinsulin 21 | VEALYLVCGERGFFYTPKTRKRREAEDLQVGQVEL | |
| | GGGPGAGSLQPLALEGSLQRKKRGIVEQCCTSICS | |
| | LYQLENYCN | |
| Exemplary | MALWMRLLPLLALLALWGPDPAAAFVNQHLCGSHL | 24 |
| preproinsulin 22 | VEALYLVCGERGFFYTPKTRKRREAEDLQVGQVEL | |
| | GGGPGAGSLQPLALEGSLQRKRRGIVEQCCTSICS | |
| | LYQLENYCN | |
| Exemplary | MALWMRLLPLLALLALWGPDPAAAFVNQHLCGSHL | 25 |
| preproinsulin 23 | VEALYLVCGERGFFYTPKTRKRREAEDLQVGQVEL | |
| | GGGPGAGSLQPLALEGSLQRRKRGIVEQCCTSICS | |
| | LYQLENYCN | |
| Exemplary | MALWMRLLPLLALLALWGPDPAAAFVNQHLCGSHL | 26 |
| preproinsulin 24 | VEALYLVCGERGFFYTPKTRKRREAEDLQVGQVEL | |
| | GGGPGAGSLQPLALEGSLQRRRRGIVEQCCTSICS | |
| | LYQLENYCN | |
| Exemplary | MALWMRLLPLLALLALWGPDPAAAFVNQHLCGSHL | 27 |
| preproinsulin 25 | VEALYLVCGERGFFYTPKTRRKREAEDLQVGQVEL | |
| | GGGPGAGSLQPLALEGSLQRHKRGIVEQCCTSICS | |
| | LYQLENYCN | |
| Exemplary | MALWMRLLPLLALLALWGPDPAAAFVNQHLCGSHL | 28 |
| preproinsulin 26 | VEALYLVCGERGFFYTPKTRRKREAEDLQVGQVEL | |
| | GGGPGAGSLQPLALEGSLQRHRRGIVEQCCTSICS | |
| | LYQLENYCN | |
| Exemplary | MALWMRLLPLLALLALWGPDPAAAFVNQHLCGSHL | 29 |
| preproinsulin 27 | VEALYLVCGERGFFYTPKTRRKREAEDLQVGQVEL | |
| | GGGPGAGSLQPLALEGSLQRKKRGIVEQCCTSICS | |
| | LYQLENYCN | |
| Exemplary | MALWMRLLPLLALLALWGPDPAAAFVNQHLCGSHL | 30 |
| preproinsulin 28 | VEALYLVCGERGFFYTPKTRRKREAEDLQVGQVEL | |
| | GGGPGAGSLQPLALEGSLQRKRRGIVEQCCTSICS | |
| | LYQLENYCN | |
| Exemplary | MALWMRLLPLLALLALWGPDPAAAFVNQHLCGSHL | 31 |
| preproinsulin 29 - | VEALYLVCGERGFFYTPKTRRKREAEDLQVGQVEL | |
| ENDSULIN101- | GGGPGAGSLQPLALEGSLQRRKRGIVEQCCTSICS | |
| Human sequence | LYQLENYCN | |
| Exemplary | MALWMRLLPLLALLALWGPDPAAAFVNQHLCGSHL | 32 |
| preproinsulin 30 | VEALYLVCGERGFFYTPKTRRKREAEDLQVGQVEL | |
| | GGGPGAGSLQPLALEGSLQRRRRGIVEQCCTSICS | |
| | LYQLENYCN | |
| Exemplary | MALWMRLLPLLALLALWGPDPAAAFVNQHLCGSHL | 33 |
| preproinsulin 31 | VEALYLVCGERGFFYTPKTRRRREAEDLQVGQVEL | |
| | GGGPGAGSLQPLALEGSLQRHKRGIVEQCCTSICS | |
| | LYQLENYCN | |
| Exemplary | MALWMRLLPLLALLALWGPDPAAAFVNQHLCGSHL | 34 |
| preproinsulin 32 | VEALYLVCGERGFFYTPKTRRRREAEDLQVGQVEL | |
| | GGGPGAGSLQPLALEGSLQRHRRGIVEQCCTSICS | |
| | LYQLENYCN | |
| Exemplary | MALWMRLLPLLALLALWGPDPAAAFVNQHLCGSHL | 35 |
| preproinsulin 33 | VEALYLVCGERGFFYTPKTRRRREAEDLQVGQVEL | |
| | GGGPGAGSLQPLALEGSLQRKKRGIVEQCCTSICS | |
| | LYQLENYCN | |
| Exemplary | MALWMRLLPLLALLALWGPDPAAAFVNQHLCGSHL | 36 |
| preproinsulin 34 | VEALYLVCGERGFFYTPKTRRRREAEDLQVGQVEL | |
| | GGGPGAGSLQPLALEGSLQRKRRGIVEQCCTSICS | |
| | LYQLENYCN | |
| Exemplary | MALWMRLLPLLALLALWGPDPAAAFVNQHLCGSHL | 37 |
| preproinsulin 35 | VEALYLVCGERGFFYTPKTRRRREAEDLQVGQVEL | |
| | GGGPGAGSLQPLALEGSLQRRKRGIVEQCCTSICS | |
| | LYQLENYCN | |
| Exemplary | MALWMRLLPLLALLALWGPDPAAAFVNQHLCGSHL | 38 |
| preproinsulin 36 | VEALYLVCGERGFFYTPKTRRRREAEDLQVGQVEL | |
| | GGGPGAGSLQPLALEGSLQRRRRGIVEQCCTSICS | |
| | LYQLENYCN | |
| Human Wildtype | MALWMRLLPLLALLALWGPDPAAAFVNQHLCGSHL | 39 |
| Preproinsulin | VEALYLVCGERGFFYTPKTRREAEDLQVGQVELGG | |
| | GPGAGSLQPLALEGSLQKRGIVEQCCTSICSLYQL | |
| | ENYCN | |
| 1994 Groskreutz- | MALWMRLLPLLALLALWGPDPAAAFVNQHLCGSHL | 40 |
| Human | VEALYLVCGERGFFYTPRTKREAEDLQVGQVELGG | |
| Preproinsulin | GPGAGSLQPLALEGSRQKRGIVEQCCTSICSLYQL | |
| | ENYCN | |
| 1992 Yanagita- | MALWMRLLPLLALLALWGPDPAAAFVNQHLCGSHL | 41 |
| Human | VEALYLVCGERGFFYTPKTRRKREDLQVGQVELGG | |
| Preproinsulin | GPGAGSLQPLALEGSRRKRGIVEQCCTSICSLYQL | |
| | ENYCN | |
| 1994 Groskreutz-Rat | MALWMRFLPLLALLVLWEPKPAQAFVKQHLCGPHL | 42 |
| preproinsulin | VEALYLVCGERGFFYTPRTKREVEDPQVPQLELGG | |
| | GPEAGDLQTLALEVARQKRGIVDQCCTSICSLYQL | |
| | ENYCN* | |
| Human Wildtype N- | MALWMRLLPLLALLALWGPDPAAA | 43 |
| terminal signal | | |
| sequence (24 AA) | | |
| Traditional 4 Amino | R - X - K/R - R* | 44 |
| Acid Furin | wherein Xis any amino acid. | |
| Consensus Sequence | | |
| Alternative 4 Amino | RX₁X₂R | 45 |
| Acid Furin | wherein X₁ is H, K, or R, and X₂ is R or K. | |
| Consensus Sequence | | |
| Canine Wildtype | MALWMRLLPLLALLALWAPAPTRAFVNQHLCGSHL | 46 |
| Preproinsulin | VEALYLVCGERGFFYTPKARREVEDLQVRDVELAG | |
| | APGEGGLQPLALEGALQKRGIVEQCCTSICSLYQL | |
| | ENYCN | |
| Exemplary variant | MALWMRLLPLLALLALWAPAPTRAFVNQHLCGSHL | 47 |
| canine preproinsulin | VEALYLVCGERGFFYTPKARRKREVEDLQVRDVEL | |
| ENDSULIN101- | AGAPGEGGLQPLALEGALQRRKRGIVEQCCTSICS | |
| Canine Preproinsulin | LYQLENYCN | |
| Exemplary Variant | MALWMRLLPLLALLALWAPAPTRAFVNQHLCGSHL | 48 |
| Canine | VEALYLVCGERGFFYTPKAXₙRX₁X₂REVEDLQVRD | |
| Preproinsulin; | VELAGAPGEGGLQPLALEGALQXₙRX₁X₂RGIVEQC | |
| Consensus based on | CTSICSLYQLENYCN | |
| ENDSULIN101- | wherein Xₙ is 0, 1, 2, 3, 4, 5, or 6 amino acids each | |
| Canine | selected from H, K, and R; wherein X₁ is H, K, or R; | |
| | and wherein X₂ is R or K. | |
| Feline Wildtype | MAPWTRLLPLLALLSLWIPAPTRAFVNQHLCGSHL | 49 |
| Preproinsulin | VEALYLVCGERGFFYTPKARREAEDLQGKDAELGE | |
| | APGAGGLQPSALEAPLQKRGIVEQCCASVCSLYQL | |
| | EHYCN | |
| Exemplary variant | MAPWTRLLPLLALLSLWIPAPTRAFVNQHLCGSHL | 50 |
| feline preproinsulin | VEALYLVCGERGFFYTPKARRKREAEDLQGKDAEL | |
| ENDSULIN101- | GEAPGAGGLQPSALEAPLQRRKRGIVEQCCASVCS | |
| Feline Preproinsulin | LYQLEHYCN | |
| Exemplary Variant | MAPWTRLLPLLALLSLWIPAPTRAFVNQHLCGSHL | 51 |
| Feline Preproinsulin; | VEALYLVCGERGFFYTPKAXₙRX₁X₂REAEDLQGKD | |
| Consensus based on | AELGEAPGAGGLQPSALEAPLQXₙRX₁X₂RGIVEQC | |
| ENDSULIN101- | CASVCSLYQLEHYCN | |
| Feline | wherein Xₙ is 0, 1, 2, 3, 4, 5, or 6 amino acids each | |
| | selected from H, K, and R; wherein X₁ is H, K, or R; | |
| | and wherein X₂ is R or K. | |
| Exemplary Variant | RHKR | 52 |
| Junction Sequence 1 | | |
| Exemplary Variant | RHRR | 53 |
| Junction Sequence 2 | | |
| Exemplary Variant | RKKR | 54 |
| Junction Sequence 3 | | |
| Exemplary Variant | RKRR | 55 |
| Junction Sequence 4 | | |
| Exemplary Variant | RRKR | 56 |
| Junction Sequence 5 | | |
| Exemplary Variant | RRRR | 57 |
| Junction Sequence 6 | | |
| Human Wildtype B- | FVNQHLCGSHLVEALYLVCGERGFFYTPKT | 59 |
| chain | | |
| Exemplary Modified | FVNQHLCGSDLVEALYLVCGERGFFYTPKT | 203 |
| Human B-Chain | | |
| Human Wildtype C- | EAEDLQVGQVELGGGPGAGSLQPLALEGSLQ | 60 |
| peptide | | |
| Human Wildtype A- | GIVEQCCTSICSLYQLENYCN | 61 |
| chain | | |
| Human, Canine, | RR | 62 |
| Feline Wildtype B/C | | |
| junction | | |
| Human, Canine, | KR | 63 |
| Feline Wildtype C/A | | |
| junction | | |
| Exemplary Variant | XₙRX₁X₂R | 64 |
| Human, Canine, | wherein Xₙ is 0, 1, 2, 3, 4, 5, or 6 amino acids each | |
| Feline junction; | selected from H, K, and R; wherein X₁ is H, K, or R; | |
| Variant B/C junction | and wherein X₂ is R or K. | |
| or | | |
| Variant C/A junction | | |
| Canine; Wildtype | MALWMRLLPLLALLALWAPAPTRA | 65 |
| N-terminal signal | | |
| sequence | | |
| Canine and Feline; | FVNQHLCGSHLVEALYLVCGERGFFYTPKA | 66 |
| Wildtype B-Chain | | |
| Canine; Wildtype | EVEDLQVRDVELAGAPGEGGLQPLALEGALQ | 67 |
| C-Peptide | | |
| Canine; Wildtype | GIVEQCCTSICSLYQLENYCN | 68 |
| A-Chain | | |
| Feline; N-terminal | MAPWTRLLPLLALLSLWIPAPTRA | 69 |
| signal sequence | | |
| Feline; Wildtype | EAEDLQGKDAELGEAPGAGGLQPSALEAPLQ | 70 |
| C-Peptide | | |
| Feline; Wildtype | GIVEQCCASVCSLYQLEHYCN | 71 |
| A-Chain | | |
| Variant Canine | FVNQHLCGSHLVEALYLVCGERGFFYTPKAXₙRX₁X₂ | 72 |
| Proinsulin; | REVEDLQVRDVELAGAPGEGGLQPLALEGALQXₙR | |
| Consensus based on | X₁X₂RGIVEQCCTSICSLYQLENYCN | |
| ENDSULIN101- | wherein Xₙ is 0, 1, 2, 3, 4, 5, or 6 amino acids each | |
| Canine | selected from H, K, and R; wherein X₁ is H, K, or R; | |
| | and wherein X₂ is R or K. | |
| Variant Feline | FVNQHLCGSHLVEALYLVCGERGFFYTPKAXₙRX₁X₂ | 73 |
| Proinsulin; | REAEDLQGKDAELGEAPGAGGLQPSALEAPLQXₙR | |
| Consensus based on | X₁X₂RGIVEQCCASVCSLYQLEHYCN | |
| ENDSULIN101- | wherein Xₙ is 0, 1, 2, 3, 4, 5, or 6 amino acids each | |
| Feline | selected from H, K, and R; wherein Xiis H, K, or R; | |
| | and wherein X₂ is R or K. | |
| ENDSULIN101- | FVNQHLCGSHLVEALYLVCGERGFFYTPKARRKRE | 74 |
| Canine Proinsulin | VEDLQVRDVELAGAPGEGGLQPLALEGALQRRKRG | |
| | IVEQCCTSICSLYQLENYCN | |
| ENDSULIN101- | FVNQHLCGSHLVEALYLVCGERGFFYTPKARRKRE | 75 |
| Feline Proinsulin | AEDLQGKDAELGEAPGAGGLQPSALEAPLQRRKRG | |
| | IVEQCCASVCSLYQLEHYCN | |
| Human | CAGCTGCGCGCTCGCTCGCTCACTGAGGCCGCCCG | 76 |
| ENDSULIN101 ITR | GGCAAAGCCCGGGCGTCGGGCGACCTTTGGTCGCC | |
| to ITR | CGGCCTCAGTGAGCGAGCGAGCGCGCAGAGAGGGA | |
| | GTGGGGTTCCTGCTATTCCCATATGATTCTAGAGC | |
| | TCGTCGACCCGCCCCCTTCACCAAGATCTTTTTGA | |
| | TGGCAGAGTTCAGTTTACCGGGTCACATTGTACCT | |
| | GGGAAGATTCAAGGATTTATGGAAAAAGTCAACAA | |
| | CAGGAGTCAGAGCAGCCGGAAAAGCATGGACTCTG | |
| | TACTTAGGACTGCGCTTTGAGCAATGGCACAGCAA | |
| | GCTTTAACCCTGTTTGCAGTCAGCACACAAACTGT | |
| | GGTTCAAAGCTCCACTTTATCTCTTCTTGTGGAAT | |
| | TCAGATATCAGATCAGTTTAAACCTTGCGGCCGCC | |
| | AGTTCTCACGTGGTGGCCACGTGCTTGGGCACTCT | |
| | AGTGCTCAAATGGGAGACAAAGAGATTAAGCTCTT | |
| | ATGTAAAATTTGCTGTTTTACATAACTTTAATGAA | |
| | TGGACAAAGTCTTGTGCATGGGGGTGGGGGTGGGG | |
| | TTAGAGGGGAACAGCTCCAGATGGCAAACATACGC | |
| | AAGGGATTTAGTCAAACAACTTTTTGGCAAAGATG | |
| | GTATGATTTTGTAATGGGGTAGGAACCAATGAAAT | |
| | GCGAGGTAAGTATGGTTAATAATCTACAGTTATTG | |
| | GTTAAAGAAGTATATTAGAGCGAGTCTTTCTGCAC | |
| | ACAGATCACCTTCCTATCAACCCCACTAGCCTCTG | |
| | GCAAAGGTACCAGCGCAGAGGCTTGGGGCAGCCGA | |
| | GCGGCAGCCAGGCCCCGGCCCGGGCCTCGGTTCCA | |
| | GAAGGGAGAGGAGCCCGCCAAGGCGCGCAAGAGAG | |
| | CGGGCTGCCTCGCAGTCCGAGCCGGAGAGGGAGCG | |
| | CGAGCCGCGCCGGCCCCGGACGGCCTCCGAAACCA | |
| | TGGCCCTGTGGATGCGCCTCCTGCCCCTGCTGGCG | |
| | CTGCTGGCCCTCTGGGGACCTGACCCAGCCGCAGC | |
| | CTTTGTGAACCAACACCTGTGCGGCTCACACCTGG | |
| | TGGAAGCTCTCTACCTAGTGTGCGGGGAACGAGGC | |
| | TTCTTCTACACACCCAAGACCCGCCGGaagagaGA | |
| | GGCAGAGGACCTGCAGGTGGGGCAGGTGGAGCTGG | |
| | GCGGGGGCCCTGGTGCAGGCAGCCTGCAGCCCTTG | |
| | GCCCTGGAGGGGTCCCTGCAGagacggAAGCGTGG | |
| | CATTGTGGAACAATGCTGTACCAGCATCTGCTCCC | |
| | TCTACCAGCTGGAGAACTACTGCAACTAGACGCAG | |
| | CCTGCAGGCAGCGTCGAGTAACATCACATTTAAAA | |
| | GCATCTCAGGTAACTATATTTTGAATTTTTTAAAA | |
| | AAGTAACTATAATAGTTATTATTAAAATAGCAAAG | |
| | ATTGACCATTTCCAAGAGCCATATAGACCAGCACC | |
| | GACCACTATTCTAAACTATTTATGTATGTAAATAT | |
| | TAGCTTTTAAAATTCTCAAAATAGTTGCTGAGTTG | |
| | GGAACCACTATTATTTCTATCGATTCAGCAGCCGT | |
| | AAGTCTAGGACAGGCTTAAATTGTTTTCACTGGTG | |
| | TAAATTGCAGAAAGATGATCTAAGTAATTTGGCAT | |
| | TTATTTTAATAGGTTTGAAAAACACATGCCATTTT | |
| | ACAAATAAGACTTATATTTGTCCTTTTGTTTTTCA | |
| | GCCTACCATGAGAATAAGAGAAAGAAAATGAAGAT | |
| | CAAAAGCTTATTCATCTGTTTTTCTTTTTCGTTGG | |
| | TGTAAAGCCAACACCCTGTCTAAAAAACATAAATT | |
| | TCTTTAATCATTTTGCCTCTTTTCTCTGTGCTTCA | |
| | ATTAATAAAAAATGGAAAGAATCTAATAGAGTGGT | |
| | ACAGCACTGTTATTTTTCAAAGATGTGTTGCTATC | |
| | CTGAAAATTCTGTAGGTTCTGTGGAAGTTCCAGTG | |
| | TTCTCTCTTATTCCACTTCGGTAGAGGATTTCTAG | |
| | TTTCTTGTGGGCTAATTAAATAAATCATTAATACT | |
| | CTTCTAAGTTATGGATTATAAACATTCAAAATAAT | |
| | ATTTTGACATTATGATAATTCTGAATAAAAGAACA | |
| | AAAACCATGGTATAGGTAAGGAATATAAAACATGG | |
| | CTTTTACCTTAGAAAAAACAATTCTAAAATTCATA | |
| | TGATTCTAGAACTAGTGGGGTACCGAATTCAGATC | |
| | GAGCATGGCTACGTAGATAAGTAGCATGGCGGGTT | |
| | AATCATTAACTACAAGGAACCCCTAGTGATGGAGT | |
| | TGGCCACTCCCTCTCTGCGCGCTCGCTCGCTCACT | |
| | GAGGCCGGGCGACCAAAGGTCGCCCGACGCCCGGG | |
| | CTTTGCCCGGGCGGCCTCAGTGAGCGAGCGAGCGC | |
| | GCAGCTGGCG | |
| Human | ATGGCCCTGTGGATGCGCCTCCTGCCCCTGCTGGC | 77 |
| ENDSULIN101 | GCTGCTGGCCCTCTGGGGACCTGACCCAGCCGCAG | |
| Encoding Sequence | CCTTTGTGAACCAACACCTGTGCGGCTCACACCTG | |
| | GTGGAAGCTCTCTACCTAGTGTGCGGGGAACGAGG | |
| | CTTCTTCTACACACCCAAGACCCGCCGGaagagaG | |
| | AGGCAGAGGACCTGCAGGTGGGGCAGGTGGAGCTG | |
| | GGCGGGGGCCCTGGTGCAGGCAGCCTGCAGCCCTT | |
| | GGCCCTGGAGGGGTCCCTGCAGagacggAAGCGTG | |
| | GCATTGTGGAACAATGCTGTACCAGCATCTGCTCC | |
| | CTCTACCAGCTGGAGAACTACTGCAACTAG | |
| Exemplary Variant | HRHKR | 78 |
| Junction Sequence 7 | | |
| Exemplary Variant | HRHRR | 79 |
| Junction Sequence 8 | | |
| Exemplary Variant | HRKKR | 80 |
| Junction Sequence 9 | | |
| Exemplary Variant | HRKRR | 81 |
| Junction Sequence | | |
| 10 | | |
| Exemplary Variant | HRRKR | 82 |
| Junction Sequence | | |
| 11 | | |
| Exemplary Variant | HRRRR | 83 |
| Junction Sequence | | |
| 12 | | |
| Exemplary Variant | KRHKR | 84 |
| Junction Sequence | | |
| 13 | | |
| Exemplary Variant | KRHRR | 85 |
| Junction Sequence | | |
| 14 | | |
| Exemplary Variant | KRKKR | 86 |
| Junction Sequence | | |
| 15 | | |
| Exemplary Variant | KRKRR | 87 |
| Junction Sequence | | |
| 16 | | |
| Exemplary Variant | KRRKR | 88 |
| Junction Sequence | | |
| 17 | | |
| Exemplary Variant | KRRRR | 89 |
| Junction Sequence | | |
| 18 | | |
| Exemplary Variant | RRHKR | 90 |
| Junction Sequence | | |
| 19 | | |
| Exemplary Variant | RRHRR | 91 |
| Junction Sequence | | |
| 20 | | |
| Exemplary Variant | RRKKR | 92 |
| Junction Sequence | | |
| 21 | | |
| Exemplary Variant | RRRKR | 93 |
| Junction Sequence | | |
| 22 | | |
| Exemplary Variant | RRKRR | 187 |
| Junction Sequence | | |
| 23 | | |
| Exemplary Variant | RRRRR | 188 |
| Junction Sequence | | |
| 24 | | |
| Exemplary Variant | HHRHKR | 94 |
| Junction Sequence | | |
| 25 | | |
| Exemplary Variant | HHRHRR | 95 |
| Junction Sequence | | |
| 26 | | |
| Exemplary Variant | HHRKKR | 96 |
| Junction Sequence | | |
| 27 | | |
| Exemplary Variant | HHRKRR | 97 |
| Junction Sequence | | |
| 28 | | |
| Exemplary Variant | HHRRKR | 98 |
| Junction Sequence | | |
| 29 | | |
| Exemplary Variant | HHRRRR | 99 |
| Junction Sequence | | |
| 30 | | |
| Exemplary Variant | HKRHKR | 100 |
| Junction Sequence | | |
| 31 | | |
| Exemplary Variant | HKRHRR | 101 |
| Junction Sequence | | |
| 32 | | |
| Exemplary Variant | HKRKKR | 102 |
| Junction Sequence | | |
| 33 | | |
| Exemplary Variant | HKRKRR | 103 |
| Junction Sequence | | |
| 34 | | |
| Exemplary Variant | HKRRKR | 104 |
| Junction Sequence | | |
| 35 | | |
| Exemplary Variant | HKRRRR | 105 |
| Junction Sequence | | |
| 36 | | |
| Exemplary Variant | HRRHKR | 106 |
| Junction Sequence | | |
| 37 | | |
| Exemplary Variant | HRRHRR | 107 |
| Junction Sequence | | |
| 38 | | |
| Exemplary Variant | HRRKKR | 108 |
| Junction Sequence | | |
| 39 | | |
| Exemplary Variant | HRRRKR | 109 |
| Junction Sequence | | |
| 40 | | |
| Exemplary Variant | KHRHKR | 110 |
| Junction Sequence | | |
| 41 | | |
| Exemplary Variant | KHRHRR | 111 |
| Junction Sequence | | |
| 42 | | |
| Exemplary Variant | KHRKKR | 112 |
| Junction Sequence | | |
| 43 | | |
| Exemplary Variant | KHRKRR | 113 |
| Junction Sequence | | |
| 44 | | |
| Exemplary Variant | KHRRKR | 114 |
| Junction Sequence | | |
| 45 | | |
| Exemplary Variant | KHRRRR | 115 |
| Junction Sequence | | |
| 46 | | |
| Exemplary Variant | KKRHKR | 116 |
| Junction Sequence | | |
| 47 | | |
| Exemplary Variant | KKRHRR | 117 |
| Junction Sequence | | |
| 48 | | |
| Exemplary Variant | KKRKKR | 118 |
| Junction Sequence | | |
| 49 | | |
| Exemplary Variant | KKRKRR | 119 |
| Junction Sequence | | |
| 50 | | |
| Exemplary Variant | KKRRKR | 120 |
| Junction Sequence | | |
| 51 | | |
| Exemplary Variant | KKRRRR | 121 |
| Junction Sequence | | |
| 52 | | |
| Exemplary Variant | KRRHKR | 122 |
| Junction Sequence | | |
| 53 | | |
| Exemplary Variant | KRRHRR | 123 |
| Junction Sequence | | |
| 54 | | |
| Exemplary Variant | KRRKKR | 124 |
| Junction Sequence | | |
| 55 | | |
| Exemplary Variant | KRRRKR | 125 |
| Junction Sequence | | |
| 56 | | |
| Exemplary Variant | RHRHKR | 126 |
| Junction Sequence | | |
| 57 | | |
| Exemplary Variant | RHRHRR | 127 |
| Junction Sequence | | |
| 58 | | |
| Exemplary Variant | RHRKKR | 128 |
| Junction Sequence | | |
| 59 | | |
| Exemplary Variant | RHRKRR | 129 |
| Junction Sequence | | |
| 60 | | |
| Exemplary Variant | RKRHKR | 130 |
| Junction Sequence | | |
| 61 | | |
| Exemplary Variant | RKRHRR | 131 |
| Junction Sequence | | |
| 62 | | |
| Exemplary Variant | RKRKKR | 132 |
| Junction Sequence | | |
| 63 | | |
| Exemplary Variant | RKRKRR | 133 |
| Junction Sequence | | |
| 64 | | |
| Exemplary Variant | RRRHKR | 134 |
| Junction Sequence | | |
| 65 | | |
| Exemplary Variant | RRRHRR | 135 |
| Junction Sequence | | |
| 66 | | |
| Exemplary Variant | RRRKKR | 136 |
| Junction Sequence | | |
| 67 | | |
| Exemplary Variant | HRRKRR | 189 |
| Junction Sequence | | |
| 68 | | |
| Exemplary Variant | HRRRRR | 190 |
| Junction Sequence | | |
| 69 | | |
| Exemplary Variant | KRRKRR | 191 |
| Junction Sequence | | |
| 70 | | |
| Exemplary Variant | KRRRRR | 192 |
| Junction Sequence | | |
| 71 | | |
| Exemplary Variant | RHRRKR | 193 |
| Junction Sequence | | |
| 72 | | |
| Exemplary Variant | RHRRRR | 194 |
| Junction Sequence | | |
| 73 | | |
| Exemplary Variant | RKRRKR | 195 |
| Junction Sequence | | |
| 74 | | |
| Exemplary Variant | RKRRRR | 196 |
| Junction Sequence | | |
| 75 | | |
| Exemplary Variant | RRRKRR | 197 |
| Junction Sequence | | |
| 76 | | |
| Exemplary Variant | RRRRKR | 198 |
| Junction Sequence | | |
| 77 | | |
| Exemplary Variant | RRRRRR | 199 |
| Junction Sequence | | |
| 78 | | |
| Wildtype _31_32 | CGERGFFYTPKTRREAEDLQ | 137 |
| Wildtype _64_65 | SLQPLALEGSLQKRGIVEQC | 138 |
| 1994 Groskreutz-Rat | CGERGFFYTPRTKREVEDPQ | 139 |
| _31_32 | | |
| 1994 Groskreutz-Rat | DLQTLALEVARQKRGIVDQC | 140 |
| _64_65 | | |
| 1994 Groskreutz- | CGERGFFYTPRTKREAEDLQ | 141 |
| Human _31_32 | | |
| 1994 Groskreutz- | SLQPLALEGSRQKRGIVEQC | 142 |
| Human _64_65 | | |
| 1992 Yanagita- | ERGFFYTPKTRRKREDLQVG | 143 |
| Human _31_32 | | |
| 1992 Yanagita- | SLQPLALEGSRRKRGIVEQC | 144 |
| Human _64_65 | | |
| ENDSULIN101_31_ | ERGFFYTPKTRRKREAEDLQ | 145 |
| 32 | | |
| ENDSULIN101_64_ | QPLALEGSLQRRKRGIVEQC | 146 |
| 65 | | |
| Exemplary | FVNQHLCGSHLVEALYLVCGERGFFYTPKTRHKRE | 147 |
| proinsulin 1 | AEDLQVGQVELGGGPGAGSLQPLALEGSLQRHKRG | |
| | IVEQCCTSICSLYQLENYCN | |
| Exemplary | FVNQHLCGSHLVEALYLVCGERGFFYTPKTRHKRE | 148 |
| proinsulin 2 | AEDLQVGQVELGGGPGAGSLQPLALEGSLQRHRRG | |
| | IVEQCCTSICSLYQLENYCN | |
| Exemplary | FVNQHLCGSHLVEALYLVCGERGFFYTPKTRHKRE | 149 |
| proinsulin 3 | AEDLQVGQVELGGGPGAGSLQPLALEGSLQRKKRG | |
| | IVEQCCTSICSLYQLENYCN | |
| Exemplary | FVNQHLCGSHLVEALYLVCGERGFFYTPKTRHKRE | 150 |
| proinsulin 4 | AEDLQVGQVELGGGPGAGSLQPLALEGSLQRKRRG | |
| | IVEQCCTSICSLYQLENYCN | |
| Exemplary | FVNQHLCGSHLVEALYLVCGERGFFYTPKTRHKRE | 151 |
| proinsulin 5 | AEDLQVGQVELGGGPGAGSLQPLALEGSLQRRKRG | |
| | IVEQCCTSICSLYQLENYCN | |
| Exemplary | FVNQHLCGSHLVEALYLVCGERGFFYTPKTRHKRE | 152 |
| proinsulin 6 | AEDLQVGQVELGGGPGAGSLQPLALEGSLQRRRRG | |
| | IVEQCCTSICSLYQLENYCN | |
| Exemplary | FVNQHLCGSHLVEALYLVCGERGFFYTPKTRHRRE | 153 |
| proinsulin 7 | AEDLQVGQVELGGGPGAGSLQPLALEGSLQRHKRG | |
| | IVEQCCTSICSLYQLENYCN | |
| Exemplary | FVNQHLCGSHLVEALYLVCGERGFFYTPKTRHRRE | 154 |
| proinsulin 8 | AEDLQVGQVELGGGPGAGSLQPLALEGSLQRHRRG | |
| | IVEQCCTSICSLYQLENYCN | |
| Exemplary | FVNQHLCGSHLVEALYLVCGERGFFYTPKTRHRRE | 155 |
| proinsulin 9 | AEDLQVGQVELGGGPGAGSLQPLALEGSLQRKKRG | |
| | IVEQCCTSICSLYQLENYCN | |
| Exemplary | FVNQHLCGSHLVEALYLVCGERGFFYTPKTRHRRE | 156 |
| proinsulin 10 | AEDLQVGQVELGGGPGAGSLQPLALEGSLQRKRRG | |
| | IVEQCCTSICSLYQLENYCN | |
| Exemplary | FVNQHLCGSHLVEALYLVCGERGFFYTPKTRHRRE | 157 |
| proinsulin 11 | AEDLQVGQVELGGGPGAGSLQPLALEGSLQRRKRG | |
| | IVEQCCTSICSLYQLENYCN | |
| Exemplary | FVNQHLCGSHLVEALYLVCGERGFFYTPKTRHRRE | 158 |
| proinsulin 12 | AEDLQVGQVELGGGPGAGSLQPLALEGSLQRRRRG | |
| | IVEQCCTSICSLYQLENYCN | |
| Exemplary | FVNQHLCGSHLVEALYLVCGERGFFYTPKTRKKRE | 159 |
| proinsulin 13 | AEDLQVGQVELGGGPGAGSLQPLALEGSLQRHKRG | |
| | IVEQCCTSICSLYQLENYCN | |
| Exemplary | FVNQHLCGSHLVEALYLVCGERGFFYTPKTRKKRE | 160 |
| proinsulin 14 | AEDLQVGQVELGGGPGAGSLQPLALEGSLQRHRRG | |
| | IVEQCCTSICSLYQLENYCN | |
| Exemplary | FVNQHLCGSHLVEALYLVCGERGFFYTPKTRKKRE | 161 |
| proinsulin 15 | AEDLQVGQVELGGGPGAGSLQPLALEGSLQRKKRG | |
| | IVEQCCTSICSLYQLENYCN | |
| Exemplary | FVNQHLCGSHLVEALYLVCGERGFFYTPKTRKKRE | 162 |
| proinsulin 16 | AEDLQVGQVELGGGPGAGSLQPLALEGSLQRKRRG | |
| | IVEQCCTSICSLYQLENYCN | |
| Exemplary | FVNQHLCGSHLVEALYLVCGERGFFYTPKTRKKRE | 163 |
| proinsulin 17 | AEDLQVGQVELGGGPGAGSLQPLALEGSLQRRKRG | |
| | IVEQCCTSICSLYQLENYCN | |
| Exemplary | FVNQHLCGSHLVEALYLVCGERGFFYTPKTRKKRE | 164 |
| proinsulin 18 | AEDLQVGQVELGGGPGAGSLQPLALEGSLQRRRRG | |
| | IVEQCCTSICSLYQLENYCN | |
| Exemplary | FVNQHLCGSHLVEALYLVCGERGFFYTPKTRKRRE | 165 |
| proinsulin 19 | AEDLQVGQVELGGGPGAGSLQPLALEGSLQRHKRG | |
| | IVEQCCTSICSLYQLENYCN | |
| Exemplary | FVNQHLCGSHLVEALYLVCGERGFFYTPKTRKRRE | 166 |
| proinsulin 20 | AEDLQVGQVELGGGPGAGSLQPLALEGSLQRHRRG | |
| | IVEQCCTSICSLYQLENYCN | |
| Exemplary | FVNQHLCGSHLVEALYLVCGERGFFYTPKTRKRRE | 167 |
| proinsulin 21 | AEDLQVGQVELGGGPGAGSLQPLALEGSLQRKKRG | |
| | IVEQCCTSICSLYQLENYCN | |
| Exemplary | FVNQHLCGSHLVEALYLVCGERGFFYTPKTRKRRE | 168 |
| proinsulin 22 | AEDLQVGQVELGGGPGAGSLQPLALEGSLQRKRRG | |
| | IVEQCCTSICSLYQLENYCN | |
| Exemplary | FVNQHLCGSHLVEALYLVCGERGFFYTPKTRKRRE | 169 |
| proinsulin 23 | AEDLQVGQVELGGGPGAGSLQPLALEGSLQRRKRG | |
| | IVEQCCTSICSLYQLENYCN | |
| Exemplary | FVNQHLCGSHLVEALYLVCGERGFFYTPKTRKRRE | 170 |
| proinsulin 24 | AEDLQVGQVELGGGPGAGSLQPLALEGSLQRRRRG | |
| | IVEQCCTSICSLYQLENYCN | |
| Exemplary | FVNQHLCGSHLVEALYLVCGERGFFYTPKTRRKRE | 171 |
| proinsulin 25 | AEDLQVGQVELGGGPGAGSLQPLALEGSLQRHKRG | |
| | IVEQCCTSICSLYQLENYCN | |
| Exemplary | FVNQHLCGSHLVEALYLVCGERGFFYTPKTRRKRE | 172 |
| proinsulin 26 | AEDLQVGQVELGGGPGAGSLQPLALEGSLQRHRRG | |
| | IVEQCCTSICSLYQLENYCN | |
| Exemplary | FVNQHLCGSHLVEALYLVCGERGFFYTPKTRRKRE | 173 |
| proinsulin 27 | AEDLQVGQVELGGGPGAGSLQPLALEGSLQRKKRG | |
| | IVEQCCTSICSLYQLENYCN | |
| Exemplary | FVNQHLCGSHLVEALYLVCGERGFFYTPKTRRKRE | 174 |
| proinsulin 28 | AEDLQVGQVELGGGPGAGSLQPLALEGSLQRKRRG | |
| | IVEQCCTSICSLYQLENYCN | |
| Exemplary | FVNQHLCGSHLVEALYLVCGERGFFYTPKTRRKRE | 175 |
| proinsulin 29 - | AEDLQVGQVELGGGPGAGSLQPLALEGSLQRRKRG | |
| ENDSULIN101- | IVEQCCTSICSLYQLENYCN | |
| Human sequence | | |
| Exemplary | FVNQHLCGSHLVEALYLVCGERGFFYTPKTRRKRE | 176 |
| proinsulin 30 | AEDLQVGQVELGGGPGAGSLQPLALEGSLQRRRRG | |
| | IVEQCCTSICSLYQLENYCN | |
| Exemplary | FVNQHLCGSHLVEALYLVCGERGFFYTPKTRRRRE | 177 |
| proinsulin 31 | AEDLQVGQVELGGGPGAGSLQPLALEGSLQRHKRG | |
| | IVEQCCTSICSLYQLENYCN | |
| Exemplary | FVNQHLCGSHLVEALYLVCGERGFFYTPKTRRRRE | 178 |
| proinsulin 32 | AEDLQVGQVELGGGPGAGSLQPLALEGSLQRHRRG | |
| | IVEQCCTSICSLYQLENYCN | |
| Exemplary | FVNQHLCGSHLVEALYLVCGERGFFYTPKTRRRRE | 179 |
| proinsulin 33 | AEDLQVGQVELGGGPGAGSLQPLALEGSLQRKKRG | |
| | IVEQCCTSICSLYQLENYCN | |
| Exemplary | FVNQHLCGSHLVEALYLVCGERGFFYTPKTRRRRE | 180 |
| proinsulin 34 | AEDLQVGQVELGGGPGAGSLQPLALEGSLQRKRRG | |
| | IVEQCCTSICSLYQLENYCN | |
| Exemplary | FVNQHLCGSHLVEALYLVCGERGFFYTPKTRRRRE | 181 |
| proinsulin 35 | AEDLQVGQVELGGGPGAGSLQPLALEGSLQRRKRG | |
| | IVEQCCTSICSLYQLENYCN | |
| Exemplary | FVNQHLCGSHLVEALYLVCGERGFFYTPKTRRRRE | 182 |
| proinsulin 36 | AEDLQVGQVELGGGPGAGSLQPLALEGSLQRRRRG | |
| | IVEQCCTSICSLYQLENYCN | |
| 1994 Groskreutz-Rat | CAGCTGCGCGCTCGCTCGCTCACTGAGGCCGCCCG | 183 |
| from ITR to ITR | GGCAAAGCCCGGGCGTCGGGCGACCTTTGGTCGCC | |
| | CGGCCTCAGTGAGCGAGCGAGCGCGCAGAGAGGGA | |
| | GTGGGGTTCCTGCTATTCCCATATGATTCTAGAGC | |
| | TCGTCGACCCGCCCCCTTCACCAAGATCTTTTTGA | |
| | TGGCAGAGTTCAGTTTACCGGGTCACATTGTACCT | |
| | GGGAAGATTCAAGGATTTATGGAAAAAGTCAACAA | |
| | CAGGAGTCAGAGCAGCCGGAAAAGCATGGACTCTG | |
| | TACTTAGGACTGCGCTTTGAGCAATGGCACAGCAA | |
| | GCTTTAACCCTGTTTGCAGTCAGCACACAAACTGT | |
| | GGTTCAAAGCTCCACTTTATCTCTTCTTGTGGAAT | |
| | TCAGATATCAGATCAGTTTAAACCTTGCGGCCGCC | |
| | AGTTCTCACGTGGTGGCCACGTGCTTGGGCACTCT | |
| | AGTGCTCAAATGGGAGACAAAGAGATTAAGCTCTT | |
| | ATGTAAAATTTGCTGTTTTACATAACTTTAATGAA | |
| | TGGACAAAGTCTTGTGCATGGGGGTGGGGGTGGGG | |
| | TTAGAGGGGAACAGCTCCAGATGGCAAACATACGC | |
| | AAGGGATTTAGTCAAACAACTTTTTGGCAAAGATG | |
| | GTATGATTTTGTAATGGGGTAGGAACCAATGAAAT | |
| | GCGAGGTAAGTATGGTTAATAATCTACAGTTATTG | |
| | GTTAAAGAAGTATATTAGAGCGAGTCTTTCTGCAC | |
| | ACAGATCACCTTCCTATCAACCCCACTAGCCTCTG | |
| | GCAAAGGTACCAGCGCAGAGGCTTGGGGCAGCCGA | |
| | GCGGCAGCCAGGCCCCGGCCCGGGCCTCGGTTCCA | |
| | GAAGGGAGAGGAGCCCGCCAAGGCGCGCAAGAGAG | |
| | CGGGCTGCCTCGCAGTCCGAGCCGGAGAGGGAGCG | |
| | CGAGCCGCGCCGGCCCCGGACGGCCTCCGAAACCA | |
| | TGGCTCTGTGGATGAGATTCCTGCCTCTGCTGGCC | |
| | CTGCTGGTGCTGTGGGAACCTAAGCCTGCCCAGGC | |
| | CTTCGTGAAGCAGCACCTGTGTGGACCCCACCTGG | |
| | TGGAAGCCCTGTACCTCGTGTGTGGCGAGAGAGGC | |
| | TTCTTCTACACCCCTAGGACCAAGAGAGAGGTGGA | |
| | AGATCCCCAGGTGCCCCAGCTGGAACTGGGCGGAG | |
| | GACCTGAAGCTGGCGACCTGCAGACACTGGCCCTG | |
| | GAAGTGGCCAGACAGAAAAGGGGCATCGTGGACCA | |
| | GTGCTGCACCAGCATCTGCAGCCTGTATCAGCTGG | |
| | AAAACTACTGCAACTGAACGCAGCCTGCAGGCAGC | |
| | GTCGAGTAACATCACATTTAAAAGCATCTCAGGTA | |
| | ACTATATTTTGAATTTTTTAAAAAAGTAACTATAA | |
| | TAGTTATTATTAAAATAGCAAAGATTGACCATTTC | |
| | CAAGAGCCATATAGACCAGCACCGACCACTATTCT | |
| | AAACTATTTATGTATGTAAATATTAGCTTTTAAAA | |
| | TTCTCAAAATAGTTGCTGAGTTGGGAACCACTATT | |
| | ATTTCTATCGATTCAGCAGCCGTAAGTCTAGGACA | |
| | GGCTTAAATTGTTTTCACTGGTGTAAATTGCAGAA | |
| | AGATGATCTAAGTAATTTGGCATTTATTTTAATAG | |
| | GTTTGAAAAACACATGCCATTTTACAAATAAGACT | |
| | TATATTTGTCCTTTTGTTTTTCAGCCTACCATGAG | |
| | AATAAGAGAAAGAAAATGAAGATCAAAAGCTTATT | |
| | CATCTGTTTTTCTTTTTCGTTGGTGTAAAGCCAAC | |
| | ACCCTGTCTAAAAAACATAAATTTCTTTAATCATT | |
| | TTGCCTCTTTTCTCTGTGCTTCAATTAATAAAAAA | |
| | TGGAAAGAATCTAATAGAGTGGTACAGCACTGTTA | |
| | TTTTTCAAAGATGTGTTGCTATCCTGAAAATTCTG | |
| | TAGGTTCTGTGGAAGTTCCAGTGTTCTCTCTTATT | |
| | CCACTTCGGTAGAGGATTTCTAGTTTCTTGTGGGC | |
| | TAATTAAATAAATCATTAATACTCTTCTAAGTTAT | |
| | GGATTATAAACATTCAAAATAATATTTTGACATTA | |
| | TGATAATTCTGAATAAAAGAACAAAAACCATGGTA | |
| | TAGGTAAGGAATATAAAACATGGCTTTTACCTTAG | |
| | AAAAAACAATTCTAAAATTCATATGATTCTAGAAC | |
| | TAGTGGGGTACCGAATTCAGATCGAGCATGGCTAC | |
| | GTAGATAAGTAGCATGGCGGGTTAATCATTAACTA | |
| | CAAGGAACCCCTAGTGATGGAGTTGGCCACTCCCT | |
| | CTCTGCGCGCTCGCTCGCTCACTGAGGCCGGGCGA | |
| | CCAAAGGTCGCCCGACGCCCGGGCTTTGCCCGGGC | |
| | GGCCTCAGTGAGCGAGCGAGCGCGCAGCTGGCG | |
| 1994 Groskreutz- | CAGCTGCGCGCTCGCTCGCTCACTGAGGCCGCCCG | 184 |
| Human from ITR to | GGCAAAGCCCGGGCGTCGGGCGACCTTTGGTCGCC | |
| ITR | CGGCCTCAGTGAGCGAGCGAGCGCGCAGAGAGGGA | |
| | GTGGGGTTCCTGCTATTCCCATATGATTCTAGAGC | |
| | TCGTCGACCCGCCCCCTTCACCAAGATCTTTTTGA | |
| | TGGCAGAGTTCAGTTTACCGGGTCACATTGTACCT | |
| | GGGAAGATTCAAGGATTTATGGAAAAAGTCAACAA | |
| | CAGGAGTCAGAGCAGCCGGAAAAGCATGGACTCTG | |
| | TACTTAGGACTGCGCTTTGAGCAATGGCACAGCAA | |
| | GCTTTAACCCTGTTTGCAGTCAGCACACAAACTGT | |
| | GGTTCAAAGCTCCACTTTATCTCTTCTTGTGGAAT | |
| | TCAGATATCAGATCAGTTTAAACCTTGCGGCCGCC | |
| | AGTTCTCACGTGGTGGCCACGTGCTTGGGCACTCT | |
| | AGTGCTCAAATGGGAGACAAAGAGATTAAGCTCTT | |
| | ATGTAAAATTTGCTGTTTTACATAACTTTAATGAA | |
| | TGGACAAAGTCTTGTGCATGGGGGTGGGGGTGGGG | |
| | TTAGAGGGGAACAGCTCCAGATGGCAAACATACGC | |
| | AAGGGATTTAGTCAAACAACTTTTTGGCAAAGATG | |
| | GTATGATTTTGTAATGGGGTAGGAACCAATGAAAT | |
| | GCGAGGTAAGTATGGTTAATAATCTACAGTTATTG | |
| | GTTAAAGAAGTATATTAGAGCGAGTCTTTCTGCAC | |
| | ACAGATCACCTTCCTATCAACCCCACTAGCCTCTG | |
| | GCAAAGGTACCAGCGCAGAGGCTTGGGGCAGCCGA | |
| | GCGGCAGCCAGGCCCCGGCCCGGGCCTCGGTTCCA | |
| | GAAGGGAGAGGAGCCCGCCAAGGCGCGCAAGAGAG | |
| | CGGGCTGCCTCGCAGTCCGAGCCGGAGAGGGAGCG | |
| | CGAGCCGCGCCGGCCCCGGACGGCCTCCGAAACCA | |
| | TGGCCCTGTGGATGCGCCTCCTGCCCCTGCTGGCG | |
| | CTGCTGGCCCTCTGGGGACCTGACCCAGCCGCAGC | |
| | CTTTGTGAACCAACACCTGTGCGGCTCACACCTGG | |
| | TGGAAGCTCTCTACCTAGTGTGCGGGGAACGAGGC | |
| | TTCTTCTACACACCCAgGACCaagCGGGAGGCAGA | |
| | GGACCTGCAGGTGGGGCAGGTGGAGCTGGGCGGGG | |
| | GCCCTGGTGCAGGCAGCCTGCAGCCCTTGGCCCTG | |
| | GAGGGGTCCCgGCAGAAGCGTGGCATTGTGGAACA | |
| | ATGCTGTACCAGCATCTGCTCCCTCTACCAGCTGG | |
| | AGAACTACTGCAACTAGACGCAGCCTGCAGGCAGC | |
| | GTCGAGTAACATCACATTTAAAAGCATCTCAGGTA | |
| | ACTATATTTTGAATTTTTTAAAAAAGTAACTATAA | |
| | TAGTTATTATTAAAATAGCAAAGATTGACCATTTC | |
| | CAAGAGCCATATAGACCAGCACCGACCACTATTCT | |
| | AAACTATTTATGTATGTAAATATTAGCTTTTAAAA | |
| | TTCTCAAAATAGTTGCTGAGTTGGGAACCACTATT | |
| | ATTTCTATCGATTCAGCAGCCGTAAGTCTAGGACA | |
| | GGCTTAAATTGTTTTCACTGGTGTAAATTGCAGAA | |
| | AGATGATCTAAGTAATTTGGCATTTATTTTAATAG | |
| | GTTTGAAAAACACATGCCATTTTACAAATAAGACT | |
| | TATATTTGTCCTTTTGTTTTTCAGCCTACCATGAG | |
| | AATAAGAGAAAGAAAATGAAGATCAAAAGCTTATT | |
| | CATCTGTTTTTCTTTTTCGTTGGTGTAAAGCCAAC | |
| | ACCCTGTCTAAAAAACATAAATTTCTTTAATCATT | |
| | TTGCCTCTTTTCTCTGTGCTTCAATTAATAAAAAA | |
| | TGGAAAGAATCTAATAGAGTGGTACAGCACTGTTA | |
| | TTTTTCAAAGATGTGTTGCTATCCTGAAAATTCTG | |
| | TAGGTTCTGTGGAAGTTCCAGTGTTCTCTCTTATT | |
| | CCACTTCGGTAGAGGATTTCTAGTTTCTTGTGGGC | |
| | TAATTAAATAAATCATTAATACTCTTCTAAGTTAT | |
| | GGATTATAAACATTCAAAATAATATTTTGACATTA | |
| | TGATAATTCTGAATAAAAGAACAAAAACCATGGTA | |
| | TAGGTAAGGAATATAAAACATGGCTTTTACCTTAG | |
| | AAAAAACAATTCTAAAATTCATATGATTCTAGAAC | |
| | TAGTGGGGTACCGAATTCAGATCGAGCATGGCTAC | |
| | GTAGATAAGTAGCATGGCGGGTTAATCATTAACTA | |
| | CAAGGAACCCCTAGTGATGGAGTTGGCCACTCCCT | |
| | CTCTGCGCGCTCGCTCGCTCACTGAGGCCGGGCGA | |
| | CCAAAGGTCGCCCGACGCCCGGGCTTTGCCCGGGC | |
| | GGCCTCAGTGAGCGAGCGAGCGCGCAGCTGGCG | |
| 1992 Yanagita- | CAGCTGCGCGCTCGCTCGCTCACTGAGGCCGCCCG | 185 |
| Human from ITR to | GGCAAAGCCCGGGCGTCGGGCGACCTTTGGTCGCC | |
| ITR | CGGCCTCAGTGAGCGAGCGAGCGCGCAGAGAGGGA | |
| | GTGGGGTTCCTGCTATTCCCATATGATTCTAGAGC | |
| | TCGTCGACCCGCCCCCTTCACCAAGATCTTTTTGA | |
| | TGGCAGAGTTCAGTTTACCGGGTCACATTGTACCT | |
| | GGGAAGATTCAAGGATTTATGGAAAAAGTCAACAA | |
| | CAGGAGTCAGAGCAGCCGGAAAAGCATGGACTCTG | |
| | TACTTAGGACTGCGCTTTGAGCAATGGCACAGCAA | |
| | GCTTTAACCCTGTTTGCAGTCAGCACACAAACTGT | |
| | GGTTCAAAGCTCCACTTTATCTCTTCTTGTGGAAT | |
| | TCAGATATCAGATCAGTTTAAACCTTGCGGCCGCC | |
| | AGTTCTCACGTGGTGGCCACGTGCTTGGGCACTCT | |
| | AGTGCTCAAATGGGAGACAAAGAGATTAAGCTCTT | |
| | ATGTAAAATTTGCTGTTTTACATAACTTTAATGAA | |
| | TGGACAAAGTCTTGTGCATGGGGGTGGGGGTGGGG | |
| | TTAGAGGGGAACAGCTCCAGATGGCAAACATACGC | |
| | AAGGGATTTAGTCAAACAACTTTTTGGCAAAGATG | |
| | GTATGATTTTGTAATGGGGTAGGAACCAATGAAAT | |
| | GCGAGGTAAGTATGGTTAATAATCTACAGTTATTG | |
| | GTTAAAGAAGTATATTAGAGCGAGTCTTTCTGCAC | |
| | ACAGATCACCTTCCTATCAACCCCACTAGCCTCTG | |
| | GCAAAGGTACCAGCGCAGAGGCTTGGGGCAGCCGA | |
| | GCGGCAGCCAGGCCCCGGCCCGGGCCTCGGTTCCA | |
| | GAAGGGAGAGGAGCCCGCCAAGGCGCGCAAGAGAG | |
| | CGGGCTGCCTCGCAGTCCGAGCCGGAGAGGGAGCG | |
| | CGAGCCGCGCCGGCCCCGGACGGCCTCCGAAACCA | |
| | TGGCCCTGTGGATGCGCCTCCTGCCCCTGCTGGCG | |
| | CTGCTGGCCCTCTGGGGACCTGACCCAGCCGCAGC | |
| | CTTTGTGAACCAACACCTGTGCGGCTCACACCTGG | |
| | TGGAAGCTCTCTACCTAGTGTGCGGGGAACGAGGC | |
| | TTCTTCTACACACCCAAGACCCGCCGGaAGagAGA | |
| | GGACCTGCAGGTGGGGCAGGTGGAGCTGGGCGGGG | |
| | GCCCTGGTGCAGGCAGCCTGCAGCCCTTGGCCCTG | |
| | GAGGGGTCCCgGCgGAAGCGTGGCATTGTGGAACA | |
| | ATGCTGTACCAGCATCTGCTCCCTCTACCAGCTGG | |
| | AGAACTACTGCAACTAGACGCAGCCTGCAGGCAGC | |
| | GTCGAGTAACATCACATTTAAAAGCATCTCAGGTA | |
| | ACTATATTTTGAATTTTTTAAAAAAGTAACTATAA | |
| | TAGTTATTATTAAAATAGCAAAGATTGACCATTTC | |
| | CAAGAGCCATATAGACCAGCACCGACCACTATTCT | |
| | AAACTATTTATGTATGTAAATATTAGCTTTTAAAA | |
| | TTCTCAAAATAGTTGCTGAGTTGGGAACCACTATT | |
| | ATTTCTATCGATTCAGCAGCCGTAAGTCTAGGACA | |
| | GGCTTAAATTGTTTTCACTGGTGTAAATTGCAGAA | |
| | AGATGATCTAAGTAATTTGGCATTTATTTTAATAG | |
| | GTTTGAAAAACACATGCCATTTTACAAATAAGACT | |
| | TATATTTGTCCTTTTGTTTTTCAGCCTACCATGAG | |
| | AATAAGAGAAAGAAAATGAAGATCAAAAGCTTATT | |
| | CATCTGTTTTTCTTTTTCGTTGGTGTAAAGCCAAC | |
| | ACCCTGTCTAAAAAACATAAATTTCTTTAATCATT | |
| | TTGCCTCTTTTCTCTGTGCTTCAATTAATAAAAAA | |
| | TGGAAAGAATCTAATAGAGTGGTACAGCACTGTTA | |
| | TTTTTCAAAGATGTGTTGCTATCCTGAAAATTCTG | |
| | TAGGTTCTGTGGAAGTTCCAGTGTTCTCTCTTATT | |
| | CCACTTCGGTAGAGGATTTCTAGTTTCTTGTGGGC | |
| | TAATTAAATAAATCATTAATACTCTTCTAAGTTAT | |
| | GGATTATAAACATTCAAAATAATATTTTGACATTA | |
| | TGATAATTCTGAATAAAAGAACAAAAACCATGGTA | |
| | TAGGTAAGGAATATAAAACATGGCTTTTACCTTAG | |
| | AAAAAACAATTCTAAAATTCATATGATTCTAGAAC | |
| | TAGTGGGGTACCGAATTCAGATCGAGCATGGCTAC | |
| | GTAGATAAGTAGCATGGCGGGTTAATCATTAACTA | |
| | CAAGGAACCCCTAGTGATGGAGTTGGCCACTCCCT | |
| | CTCTGCGCGCTCGCTCGCTCACTGAGGCCGGGCGA | |
| | CCAAAGGTCGCCCGACGCCCGGGCTTTGCCCGGGC | |
| | GGCCTCAGTGAGCGAGCGAGCGCGCAGCTGGCG | |
| Negative Control | CAGCTGCGCGCTCGCTCGCTCACTGAGGCCGCCCG | 186 |
| from ITR to ITR | GGCAAAGCCCGGGCGTCGGGCGACCTTTGGTCGCC | |
| | CGGCCTCAGTGAGCGAGCGAGCGCGCAGAGAGGGA | |
| | GTGGGGTTCCTGCTATTCCCATATGATTCTAGAGC | |
| | TCGTCGACCCGCCCCCTTCACCAAGATCTTTTTGA | |
| | TGGCAGAGTTCAGTTTACCGGGTCACATTGTACCT | |
| | GGGAAGATTCAAGGATTTATGGAAAAAGTCAACAA | |
| | CAGGAGTCAGAGCAGCCGGAAAAGCATGGACTCTG | |
| | TACTTAGGACTGCGCTTTGAGCAATGGCACAGCAA | |
| | GCTTTAACCCTGTTTGCAGTCAGCACACAAACTGT | |
| | GGTTCAAAGCTCCACTTTATCTCTTCTTGTGGAAT | |
| | TCAGATATCAGATCAGTTTAAACCTTGCGGCCGCC | |
| | AGTTCTCACGTGGTGGCCACGTGCTTGGGCACTCT | |
| | AGTGCTCAAATGGGAGACAAAGAGATTAAGCTCTT | |
| | ATGTAAAATTTGCTGTTTTACATAACTTTAATGAA | |
| | TGGACAAAGTCTTGTGCATGGGGGTGGGGGTGGGG | |
| | TTAGAGGGGAACAGCTCCAGATGGCAAACATACGC | |
| | AAGGGATTTAGTCAAACAACTTTTTGGCAAAGATG | |
| | GTATGATTTTGTAATGGGGTAGGAACCAATGAAAT | |
| | GCGAGGTAAGTATGGTTAATAATCTACAGTTATTG | |
| | GTTAAAGAAGTATATTAGAGCGAGTCTTTCTGCAC | |
| | ACAGATCACCTTCCTATCAACCCCACTAGCCTCTG | |
| | GCAAAGGTACCAGCGCAGAGGCTTGGGGCAGCCGA | |
| | GCGGCAGCCAGGCCCCGGCCCGGGCCTCGGTTCCA | |
| | GAAGGGAGAGGAGCCCGCCAAGGCGCGCAAGAGAG | |
| | CGGGCTGCCTCGCAGTCCGAGCCGGAGAGGGAGCG | |
| | CGAGCCGCGCCGGCCCCGGACGGCCTCCGAAACCt | |
| | TGGCCCTGTGGtTGCGCCTCCTGCCCCTGCTGGCG | |
| | CTGCTGGCCCTCTGGGGACCTGACCCAGCCGCAGC | |
| | CTTTGTGAACCAACACCTGTGCGGCTCACACCTGG | |
| | TGGAAGCTCTCTACCTAGTGTGCGGGGAACGAGGC | |
| | TTCTTCTACACACCCAgGACCaagCGGGAGGCAGA | |
| | GGACCTGCAGGTGGGGCAGGTGGAGCTGGGCGGGG | |
| | GCCCTGGTGCAGGCAGCCTGCAGCCCTTGGCCCTG | |
| | GAGGGGTCCCgGCAGAAGCGTGGCATTGTGGAACA | |
| | ATGCTGTACCAGCATCTGCTCCCTCTACCAGCTGG | |
| | AGAACTACTGCAACTAGACGCAGCCTGCAGGCAGC | |
| | GTCGAGTAACATCACATTTAAAAGCATCTCAGGTA | |
| | ACTATATTTTGAATTTTTTAAAAAAGTAACTATAA | |
| | TAGTTATTATTAAAATAGCAAAGATTGACCATTTC | |
| | CAAGAGCCATATAGACCAGCACCGACCACTATTCT | |
| | AAACTATTTATGTATGTAAATATTAGCTTTTAAAA | |
| | TTCTCAAAATAGTTGCTGAGTTGGGAACCACTATT | |
| | ATTTCTATCGATTCAGCAGCCGTAAGTCTAGGACA | |
| | GGCTTAAATTGTTTTCACTGGTGTAAATTGCAGAA | |
| | AGATGATCTAAGTAATTTGGCATTTATTTTAATAG | |
| | GTTTGAAAAACACATGCCATTTTACAAATAAGACT | |
| | TATATTTGTCCTTTTGTTTTTCAGCCTACCATGAG | |
| | AATAAGAGAAAGAAAATGAAGATCAAAAGCTTATT | |
| | CATCTGTTTTTCTTTTTCGTTGGTGTAAAGCCAAC | |
| | ACCCTGTCTAAAAAACATAAATTTCTTTAATCATT | |
| | TTGCCTCTTTTCTCTGTGCTTCAATTAATAAAAAA | |
| | TGGAAAGAATCTAATAGAGTGGTACAGCACTGTTA | |
| | TTTTTCAAAGATGTGTTGCTATCCTGAAAATTCTG | |
| | TAGGTTCTGTGGAAGTTCCAGTGTTCTCTCTTATT | |
| | CCACTTCGGTAGAGGATTTCTAGTTTCTTGTGGGC | |
| | TAATTAAATAAATCATTAATACTCTTCTAAGTTAT | |
| | GGATTATAAACATTCAAAATAATATTTTGACATTA | |
| | TGATAATTCTGAATAAAAGAACAAAAACCATGGTA | |
| | TAGGTAAGGAATATAAAACATGGCTTTTACCTTAG | |
| | AAAAAACAATTCTAAAATTCATATGATTCTAGAAC | |
| | TAGTGGGGTACCGAATTCAGATCGAGCATGGCTAC | |
| | GTAGATAAGTAGCATGGCGGGTTAATCATTAACTA | |
| | CAAGGAACCCCTAGTGATGGAGTTGGCCACTCCCT | |
| | CTCTGCGCGCTCGCTCGCTCACTGAGGCCGGGCGA | |
| | CCAAAGGTCGCCCGACGCCCGGGCTTTGCCCGGGC | |
| | GGCCTCAGTGAGCGAGCGAGCGCGCAGCTGGCG | |
| Human Wildtype | FVNQHLCGSHLVEALYLVCGERGFFYTPKTRREAE | 200 |
| Proinsulin | DLQVGQVELGGGPGAGSLQPLALEGSLQKRGIVEQ | |
| | CCTSICSLYQLENYCN | |
| Canine Wildtype | FVNQHLCGSHLVEALYLVCGERGFFYTPKARREVE | 201 |
| Proinsulin | DLQVRDVELAGAPGEGGLQPLALEGALQKRGIVEQ | |
| | CCTSICSLYQLENYCN | |
| Feline Wildtype | FVNQHLCGSHLVEALYLVCGERGFFYTPKARREAE | 202 |
| Proinsulin | DLQGKDAELGEAPGAGGLQPSALEAPLQKRGIVEQ | |
| | CCASVCSLYQLEHYCN | |

### DESCRIPTION OF CERTAIN EXEMPLARY EMBODIMENTS

### A. Overview

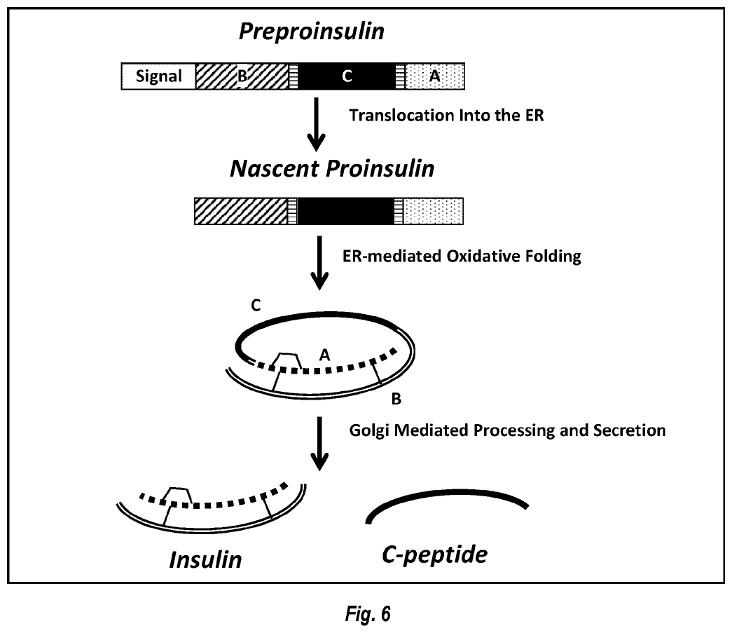

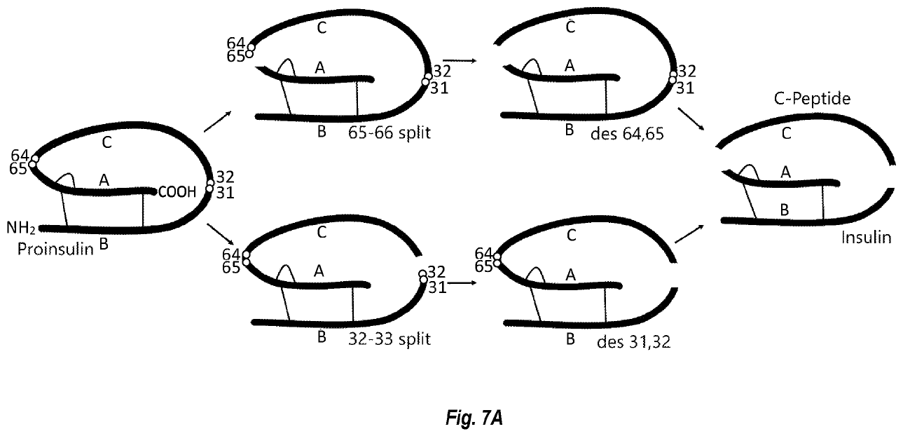

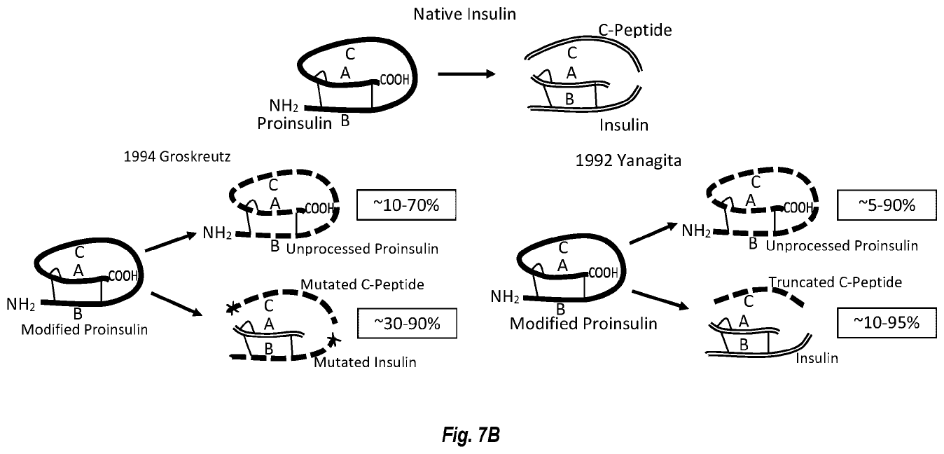

The present disclosure is directed to variant preproinsulin proteins that can be processed by protease enzymes to produce fully processed wildtype human insulin and C-peptide. As is described in Table 1 and the Examples, a number of variant preproinsulin proteins were designed that each contain variant B/C and C/A junctions comprising an enzymatic cleavage site to induce the proper processing into the desired insulin products. Preproinsulin is a biologically, nearly inactive precursor to insulin. Preproinsulin is converted into proinsulin by signal peptidases, which remove its signal peptide from its N-terminus. The process is described in FIGS. 6 and 7A. Ultimately, proinsulin is processed into the bioactive hormone insulin by proteolytic removal of the C-peptide.

The variant preproinsulin proteins and constructs encoding the same of the present disclosure may be used to treat human or non-human animals, such as dogs and cats. In some embodiments, the constructs may encode variant rat preproinsulin proteins which are then used to treat other species of animals, for example, dogs, and cats, in which rat insulin is tolerated by the recipient animal.

This disclosure provides nucleic acid molecules comprising a nucleic acid sequence encoding a variant preproinsulin polypeptide comprising an N-terminal signal sequence, a wildtype B chain or full-length variant thereof comprising at least one amino acid substitution, a variant B/C junction, a wildtype C-peptide or full-length variant thereof comprising at least one amino acid substitution, a variant C/A junction, and a wildtype A-chain or full-length variant thereof comprising at least one amino acid substitution, wherein the variant B/C junction and the variant C/A junction each comprise at least 4 amino acids selected from histidine, lysine, and arginine, wherein the variant B/C junction and the variant C/A junction each comprise an enzymatic cleavage site for a target proteolytic enzyme to cleave immediately after the C-terminal amino acid of the variant junction.

Additionally, this disclosure provides variant preproinsulin polypeptides comprising an N-terminal signal sequence, a wildtype B-chain or full-length variant thereof comprising at least one amino acid substitution, a variant B/C junction, a wildtype C-peptide or full-length variant thereof comprising at least one amino acid substitution, a variant C/A junction, and a wildtype A-chain or full-length variant thereof comprising at least one amino acid substitution, wherein the variant B/C junction and the variant C/A junction each comprise at least 4 amino acids selected from histidine, lysine, and arginine, wherein the variant B/C junction and the variant C/A junction each comprise an enzymatic cleavage site for a target proteolytic enzyme to cleave immediately after the C-terminal amino acid of the variant junction.

In some embodiments, the preproinsulin polypeptide is capable of being processed into a mature wildtype insulin protein or full-length variant thereof comprising at least one amino acid substitution and a mature wildtype C-peptide or full-length variant thereof comprising at least one amino acid substitution. In further embodiments, the preproinsulin polypeptide is capable of being processed by furin and a carboxypeptidase. into a mature wildtype insulin protein or full-length variant thereof comprising at least one amino acid substitution and a mature wildtype C-peptide or full-length variant thereof comprising at least one amino acid substitution

In some embodiments, the preproinsulin polypeptide is capable of being processed into a mature wildtype human insulin protein and a mature wildtype human C-peptide. In some embodiments, the preproinsulin polypeptide is capable of being processed into a mature wildtype canine insulin protein and a mature wildtype canine C-peptide. In some embodiments, the preproinsulin polypeptide is capable of being processed into a mature wildtype feline insulin protein and a mature wildtype feline C-peptide.

This disclosure also provides pharmaceutical compositions comprising a nucleic acid molecule described herein, a variant preproinsulin polypeptide described herein, a vector described herein, and/or a cultured host cell described herein and a pharmaceutically acceptable carrier.

For the convenience of the reader, the following definitions of terms used herein are provided.

As used herein, numerical terms are calculated based upon scientific measurements and, thus, are subject to appropriate measurement error. In some instances, a numerical term may include numerical values that are rounded to the nearest significant figure.

As used herein, “a” or “an” means “at least one” or “one or more” unless otherwise specified. As used herein, the term “or” means “and/or” unless specified otherwise. In the context of a multiple dependent claim, the use of “or” when referring back to other claims refers to those claims in the alternative only.

### B. Exemplary Nucleic Acid Molecules and Polypeptides

As used herein, “nucleic acid molecule” or “polynucleotide” are used interchangeably herein to refer to a polymer of nucleotides. A nucleotide is composed of a base, specifically a purine or pyrimidine base (i.e., cytosine (C), guanine (G), adenine (A), thymine (T) or uracil (U)); a sugar (i.e., deoxyribose or ribose); and a phosphate group. A nucleic acid molecule may be described by the nucleotide sequence representing its primary linear structure. A nucleotide sequence is typically represented from 5′ to 3′. Nucleic acid molecules include, for example, deoxyribonucleic acid (DNA) including genomic DNA, mitochondrial DNA, methylated DNA, and the like; ribonucleic acid (RNA), including messenger RNA (mRNA), small interfering RNA (siRNA), microRNA (miRNA), non-coding RNAs. A nucleic acid molecule can be single-stranded or double-stranded DNA or RNA. Alternatively, a nucleic acid molecule may be a DNA-RNA duplex.

A nucleotide base may be represented using the International Union of Pure and Applied Chemistry (IUPAC) nucleotide code shown in Table 2.

TABLE 2

| IUPAC | |
|---|---|
| nucleotide code | Base |
| A | Adenine |
| C | Cytosine |
| G | Guanine |
| T | Thymine |
| (or U) | (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |

A nucleic acid molecule or polypeptide described herein may be from any source unless otherwise indicated. The source may be a vertebrate source, including mammals such as primates (e.g., humans or cynomolgus monkeys), rodents (e.g., mice and rats), etc.

“Amino acid sequence,” means a sequence of amino acids residues in a polypeptide or protein. The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“Wildtype polypeptide,” as used herein, refers to a non-mutated version of a polypeptide that occurs in nature, or a fragment thereof. A wildtype polypeptide may be produced recombinantly.

“Variant polypeptide,” as used herein, refers to a polypeptide that differs from a reference polypeptide by a single or multiple non-native amino acid substitutions, deletions, and/or additions. In some embodiments, a variant polypeptide retains at least one biological activity of the reference polypeptide (e.g., a corresponding wildtype polypeptide). A variant polypeptide includes, for instance, polypeptides wherein one or more amino acid residues are added, deleted, within the polypeptide or at the N- or C-terminus of the polypeptide.

In some embodiments, a variant polypeptide has at least 1, 2, 3, 4, 5, or 6 amino acid substitutions compared to a reference polypeptide. In some embodiments, the variant polypeptide has at least one amino acid substitution compared to a reference polypeptide. In some embodiments, the variant polypeptide has at least two amino acid substitutions compared to a reference polypeptide. In some embodiments, the variant polypeptide has at least three amino acid substitutions compared to a reference polypeptide. In some embodiments, the variant polypeptide has at least four amino acid substitutions compared to a reference polypeptide. In some embodiments, the variant polypeptide has at least five amino acid substitutions compared to a reference polypeptide. In some embodiments, the variant polypeptide has at least six amino acid substitutions compared to a reference polypeptide. In some embodiments, the variant polypeptide has one amino acid substitution compared to a reference polypeptide. In some embodiments, the variant polypeptide has two amino acid substitutions compared to a reference polypeptide. In some embodiments, the variant polypeptide has three amino acid substitutions compared to a reference polypeptide. In some embodiments, the variant polypeptide has four amino acid substitutions compared to a reference polypeptide. In some embodiments, the variant polypeptide has five amino acid substitutions compared to a reference polypeptide. In some embodiments, the variant polypeptide has six amino acid substitutions compared to a reference polypeptide.

In some embodiments, a variant has at least about 50% sequence identity with the reference nucleic acid molecule or polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, deleted, at the N- or C-terminus of the polypeptide. In some embodiments, a variant has at least about 50% sequence identity, at least about 60% sequence identity, at least about 65% sequence identity, at least about 70% sequence identity, at least about 75% sequence identity, at least about 80% sequence identity, at least about 85% sequence identity, at least about 90% sequence identity, at least about 95% sequence identity, at least about 97% sequence identity, at least about 98% sequence identity, or at least about 99% sequence identity with the sequence of the reference nucleic acid or polypeptide.

As used herein, “percent (%) amino acid sequence identity” and “homology” with respect to a peptide, polypeptide, or antibody sequence are defined as the percentage of amino acid residues in a candidate sequence that are identical with the amino acid residues in the specific peptide or polypeptide sequence, after aligning the sequences and introducing gaps, if necessary to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALINE™ (DNASTAR) software. Those skilled in the art can determine appropriate parameters for measuring alignment, including any algorithms needed to achieve maximal alignment over the full length of sequences being compared.

An “amino acid substitution” refers to the replacement of one amino acid in a polypeptide with another amino acid. In some embodiments, an amino acid substitution is a conservative substitution. Non-limiting exemplary conservative amino acid substitutions are shown in Table 3. Amino acid substitutions may be introduced into a molecule of interest and the products screened for a desired activity, for example, retained/improved activity, decreased immunogenicity, or improved or enhanced pharmacokinetics.

TABLE 3

| Original | |
|---|---|
| Residue | Exemplary Substitutions |
| Ala (A) | Val; Leu; Ile |
| Arg (R) | Lys; Gln; Asn |
| Asn (N) | Gln; His; Asp; Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may also be grouped and substituted according to common side chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes with another class.

As used herein, the term “insulin” refers to a mature, active hormone that is a heteroduplex polypeptide comprising an A-chain and a B-chain linked together by disulfide bonds. Insulin may be from any vertebrate source, including mammals such as primates (e.g., humans and cynomolgus monkeys), rodents (e.g., mice and rats), and companion animals (e.g., dogs, cats, and horses), unless otherwise indicated. “Insulin” includes wildtype insulin and variant insulin polypeptides that substantially retain at least one biological activity of a wildtype insulin protein.

The present invention provides preproinsulin nucleic acid constructs which are processed into: (a) a signal peptide; (b) a B-chain; (c) a C-peptide; (d) an A-chain. Embedded within the construct are: (a) furin or endoprotease cleavage sites; (b) a Carboxypeptidase E cleavage sites; and (c) a signal peptide cleavage site.

In some embodiments, insulin is a human insulin, such as a wildtype human insulin or a variant human insulin. In some embodiments, a wildtype human insulin is a 51 amino acid heteroduplex comprising the A-chain of SEQ ID NO: 61 and the B-chain of SEQ ID NO: 59.

In some embodiments, insulin is a dog insulin, such as a wildtype dog insulin or a variant dog insulin. In some embodiments, a wildtype dog insulin is a 51 amino acid heteroduplex comprising the A-chain of SEQ ID NO: 68 and the B-chain of SEQ ID NO: 66.

In some embodiments, insulin is a cat insulin, such as a wildtype cat insulin or a variant cat insulin. In some embodiments, a wildtype cat insulin is a 51 amino acid heteroduplex comprising the A-chain of SEQ ID NO: 71 and the B-chain of SEQ ID NO: 66.

In some embodiments, the wildtype A-chain is a wildtype human A-chain, a wildtype canine A-chain, or a wildtype feline A-chain. In some embodiments, the wildtype A-chain comprises an amino acid sequence of SEQ ID NO: 61, SEQ ID NO: 68, or SEQ ID NO: 71. In some embodiments, the wildtype A-chain is a wildtype human A-chain. In some embodiments, the wildtype A-chain comprises an amino acid sequence of SEQ ID NO: 61. In some embodiments, the A-chain is a full-length variant A-chain comprising at least one amino acid substitution compared to a wildtype A-chain, such as compared to a wildtype human A-chain, wildtype canine A-chain, or a wildtype feline A-chain.

In some embodiments, the wildtype B-chain is a wildtype human B-chain, a wildtype canine B-chain, or a wildtype feline B-chain. In some embodiments, the wildtype B-chain comprises an amino acid sequence of SEQ ID NO: 59 or SEQ ID NO: 66. In some embodiments, the wildtype B-chain is a wildtype human B-chain. In some embodiments, the wildtype B-chain or full-length variant thereof comprises an amino acid sequence of SEQ ID NO: 59 or SEQ ID NO: 203. In some embodiments, the B-chain is a full-length variant B-chain comprising at least one amino acid substitution compared to a wildtype B-chain, such as compared to a wildtype human B-chain, wildtype canine B-chain, or a wildtype feline B-chain. In some embodiments, the B-chain comprises a histidine (H) to aspartic acid (D) substitution at position 10.

As used herein, “preproinsulin” refers a polypeptide (NH₂— Signal Peptide-B chain-B/C junction-C-peptide-C/A junction-A-chain-COOH; see FIG. 6), which may be sequentially processed into proinsulin, and finally insulin. Preproinsulin may be from any vertebrate source, including mammals such as primates (e.g., humans and cynomolgus monkeys), rodents (e.g., mice and rats), and companion animals (e.g., dogs, cats, and horses), unless otherwise indicated. Preproinsulin includes wildtype preproinsulin polypeptides and variant preproinsulin polypeptides at least some percentage of which are capable of being processed into proinsulin.

In one embodiment, preproinsulin comprises a histidine (H) to aspartic acid (D) substitution at position 10 of the B-chain, which is believed to increase the stability of mature insulin and increase insulin's affinity for its receptor. Groskreutz et al. (1994).

One aspect of the present disclosure is the cleavage sites and locations of the amino acids removed by protease enzymes for preproinsulin processing. In vivo, preproinsulin is translocated via SRP/SRP-R into the endoplasmic reticulum where it is cleaved into nascent proinsulin. The nascent proinsulin then undergoes endoplasmic reticulum-mediated oxidating folding and subsequent trans-Golgi processing to yield mature insulin and C-peptide.

The first processing step of a preproinsulin is the proteolytic elimination of the N-terminal signal peptide, which serves as a hydrophobic signal sequence for the transfer of the resulting chain through the membrane of the rough endoplasmic reticulum. In wildtype human preproinsulin, the length of the signal peptide is 24 amino acids (SEQ ID NO: 43).

In some embodiments, the N-terminal signal sequence comprises a wildtype N-terminal signal sequence. In some embodiments, the N-terminal signal sequence comprises a wildtype human N-terminal signal sequence, a wildtype canine N-terminal signal sequence, or a wildtype feline N-terminal signal sequence. In some embodiments, the N terminal signal sequence comprises an amino acid sequence of SEQ ID NO: 43, SEQ ID NO: 65, or SEQ ID NO: 69. In some embodiments, the N-terminal signal sequence comprises a wildtype human N-terminal signal sequence. In some embodiments, the N-terminal signal sequence comprises an amino acid sequence of SEQ ID NO: 43. In some embodiments, the N-terminal signal sequence comprises a variant N-terminal signal sequence.

In some embodiments, the variant preproinsulin polypeptide comprises an amino acid sequence of SEQ ID NO: 1, SEQ ID NO: 48, or SEQ ID NO: 51. In some embodiments, the variant preproinsulin polypeptide comprises an amino acid sequence of SEQ ID NO: 1. In some embodiments, the variant preproinsulin polypeptide comprises an amino acid sequence of SEQ ID NO: 48. In some embodiments, the variant preproinsulin polypeptide comprises an amino acid sequence of SEQ ID NO: 51.

In some embodiments, the variant preproinsulin polypeptide comprises an amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 47, or SEQ ID NO: 50. In some embodiments, the variant preproinsulin polypeptide comprises an amino acid sequence of SEQ ID NO: 31.

This disclosure provides a nucleic acid molecule comprising a nucleic acid sequence encoding the variant preproinsulin polypeptide as described herein. For example, this disclosure provides a nucleic acid molecule comprising a nucleic acid sequence encoding a variant preproinsulin polypeptide comprising an amino acid sequence of SEQ ID NO: 1, SEQ ID NO: 48, or SEQ ID NO: 51.

This disclosure also provides an nucleic acid molecule comprising a nucleic acid sequence encoding a variant preproinsulin polypeptide comprising an amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 47, or SEQ ID NO: 50.

This disclosure provides a nucleic acid molecule encoding a variant preproinsulin polypeptide comprising an amino acid sequence of SEQ ID NO: 31.

This disclosure further provides a nucleic acid molecule comprising a nucleic acid sequence of SEQ ID NO: 77.

In some embodiments, the preproinsulin polypeptide is capable of being processed into a proinsulin polypeptide comprising an amino acid sequence of SEQ ID NO: 2, SEQ ID NO: 72, or SEQ ID NO: 73. In some embodiments, the preproinsulin polypeptide is capable of being processed into a proinsulin polypeptide comprising an amino acid sequence of SEQ ID NO: 2.

In some embodiments, the preproinsulin polypeptide is capable of being processed into a proinsulin polypeptide comprising an amino acid sequence of SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 74, or SEQ ID NO: 75. In some embodiments, the preproinsulin polypeptide is capable of being processed into a proinsulin polypeptide comprising an amino acid sequence of SEQ ID NO: 175.

As used herein, “proinsulin” refers to a polypeptide comprising the B-chain, B/C junction, C-peptide, C/A junction, and A-chain of insulin, in order from the N-terminal to the C-terminal end. Proinsulin may be from any vertebrate source, including mammals such as primates (e.g., humans and cynomolgus monkeys), rodents (e.g., mice and rats), and companion animals (e.g., dogs, cats, and horses), unless otherwise indicated. Proinsulin includes wildtype proinsulin polypeptides and variant proinsulin polypeptides at least some percentage of which are capable of being processed into insulin and a C-peptide.

In one embodiment, histidine (H) is mutated to aspartic acid (D) at position 10 in the B-chain, which is believed to increase the stability of mature insulin and increase insulin's affinity for its receptor. Groskreutz et al. (1994).

For example, wildtype human proinsulin (SEQ ID NO: 200) comprises a B-chain (SEQ ID NO: 59), a C-peptide (SEQ ID NO: 60), and an A-chain (SEQ ID NO: 61).

As another example, wildtype canine proinsulin (SEQ ID NO: 201) comprises a B-chain (SEQ ID NO: 66), a C-peptide (SEQ ID NO: 67), and an A-chain (SEQ ID NO: 68).

As a further example, wildtype feline proinsulin (SEQ ID NO: 202) comprises a B-chain (SEQ ID NO: 66), a C-peptide (SEQ ID NO: 70), and an A-chain (SEQ ID NO: 71).

In some embodiments, the wildtype C-peptide is a wildtype human C-peptide, a wildtype canine C-peptide, or a wildtype feline C-peptide. In some embodiments, the wildtype C-peptide comprises an amino acid sequence of SEQ ID NO: 60, SEQ ID NO: 67, or SEQ ID NO: 70. In some embodiments, the wildtype C-peptide is a wildtype human C-peptide. In some embodiments, the wildtype C-peptide comprises an amino acid sequence of SEQ ID NO: 60. In some embodiments, the C-peptide is a full-length variant C-peptide comprising at least one amino acid substitution compared to a wildtype C-peptide, such as compared to a wildtype human C-peptide, wildtype canine C-peptide, or a wildtype feline C-peptide.

In some embodiments, the wildtype B-chain is a wildtype human B-chain, a wildtype canine B-chain, or a wildtype feline B-chain. In some embodiments, the wildtype B-chain comprises an amino acid sequence of SEQ ID NO: 59, or SEQ ID NO: 66. In some embodiments, the wildtype B-chain is a wildtype human B-chain. In some embodiments, the wildtype B-chain comprises an amino acid sequence of SEQ ID NO: 59. In some embodiments, the B-chain is a full-length variant B-chain comprising at least one amino acid substitution compared to a wildtype B-chain, such as compared to a wildtype human B-chain, wildtype canine B-chain, or a wildtype feline B-chain. In some embodiments, the B-chain comprises a histidine (H) to aspartic acid (D) substitution at position 10.

In some embodiments, the wildtype A-chain is a wildtype human A-chain, a wildtype canine A-chain, or a wildtype feline A-chain. In some embodiments, the wildtype A-chain comprises an amino acid sequence of SEQ ID NO: 61, SEQ ID NO: 68, or SEQ ID NO: 71. In some embodiments, the wildtype A-chain is a wildtype human A-chain. In some embodiments, the wildtype A-chain comprises an amino acid sequence of SEQ ID NO: 61. In some embodiments, the A-chain is a full-length variant A-chain comprising at least one amino acid substitution compared to a wildtype A-chain, such as compared to a wildtype human A-chain, wildtype canine A-chain, or a wildtype feline A-chain.

In some embodiments, the proinsulin polypeptide comprises the amino acid sequence of SEQ ID NO: SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 74, or SEQ ID NO: 75.

This disclosure provides an nucleic acid molecule comprising a nucleic acid sequence encoding a proinsulin polypeptide comprising an amino acid sequence of SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 74, or SEQ ID NO: 75.

Further, this disclosure provides a nucleic acid molecule comprising a nucleic acid sequence encoding a proinsulin polypeptide comprising an amino acid sequence of SEQ ID NO: 175.

In some embodiments, the preproinsulin or proinsulin construct includes two enzymatic cleavage sites. In some embodiments, the enzymatic cleavage sites are subtilisin-like proprotein convertase cleavage sites. Bergeron, et al. (2000).

In various embodiments, the disclosure provides an enzyme recognition site comprising an amino acid sequence comprising between 4 and 20 amino acids, wherein the last 4 amino acids of the recognition site generate a 4 amino acid furin cleavage site, and wherein those four amino acids are selected from the basic amino acids histidine (H), lysine (K), and arginine (R). In some embodiments, the invention provides an enzyme recognition site comprising an amino acid sequence comprising between 4 and 20 amino acids, wherein the last 4 amino acids of the recognition site generate the only furin cleavage site of the recognition site, and wherein the amino acids are selected from basic amino acids histidine (H), lysine (K), and arginine (R). In some embodiments, furin cleaves at the recognition site, and carboxypeptidase follows furin to remove any remaining C-terminal 4-10 basic amino acids. Iwaki, et al. (2021). In some embodiments, the carboxypeptidase targets basic amino acids. Skidgel (1988). In further embodiments, variant junctions comprise amino acids selected from lysine (K) and arginine (R).

As used herein, the term “B/C junction” means the amino acids located between the B-chain and C-peptide. A B/C junction may be from any vertebrate source, including mammals such as primates (e.g., humans and cynomolgus monkeys), rodents (e.g., mice and rats), and companion animals (e.g., dogs, cats, and horses), unless otherwise indicated. B/C junction includes wildtype B/C junctions and variant B/C junctions that are capable of being cleaved by a target proteolytic enzyme.

In some embodiments, a variant B/C junction comprises between 4 and 10 basic amino acids, selected from histidine (“His” or “H”), lysine (“Lys” or “K”), and arginine (“Arg” or “K”), wherein the four C-terminal amino acids each generate a four amino acid furin cleavage site. In some embodiments, a variant B/C junction comprises a four amino acid furin cleavage site.

As an example, the human B/C junction may comprise a wildtype B/C junction comprising SEQ ID NO: 62. As an example, the canine B/C junction may comprise a wildtype B/C junction comprising SEQ ID NO: 62. As an example, the feline B/C junction may comprise a wildtype B/C junction comprising SEQ ID NO: 62.

As used herein, the term “C/A junction” means the amino acids located between the C-peptide and the A-chain. A C/A junction may be from any vertebrate source, including mammals such as primates (e.g., humans and cynomolgus monkeys), rodents (e.g., mice and rats), and companion animals (e.g., dogs, cats, and horses), unless otherwise indicated. C/A junction includes wildtype C/A junctions and variant C/A junctions that are capable of being cleaved by a target proteolytic enzyme.

As an example, the human C/A junction may comprise a wildtype C/A junction comprising SEQ ID NO: 63. As an example, the canine C/A junction may comprise a wildtype C/A junction comprising SEQ ID NO: 63. As an example, the feline C/A junction may comprise a wildtype C/A junction comprising SEQ ID NO: 63.

In some embodiments, a variant C/A junction comprises between 4 and 10 basic amino acids, selected from histidine (“His” or “H”), lysine (“Lys” or “K”), and arginine (“Arg” or “K”), wherein the four C-terminal amino acids each generate a four amino acid furin cleavage site. In some embodiments, a variant C/A junction comprises a four amino acid furin cleavage site.

As used herein, the term “enzymatic cleavage site” means a portion of a polypeptide that is recognized by and cleaved by a proteolytic enzyme.

In some embodiments, the enzymatic cleavage site is a subtilisin-like proprotein convertase cleavage site. In some embodiments, the enzymatic cleavage site is a furin cleavage site.

As used herein, the term “furin cleavage site” means a portion of a polypeptide that is recognized by and cleaved by furin. A furin cleavage site may be referred to by a four amino acid recognition sequence or by an expanded 20 amino acid furin consensus cleavage site.

In some embodiments, the enzymatic cleavage site comprises an amino acid sequence of RX₁X₂R (SEQ ID NO: 45), wherein X₁ is histidine, lysine, or arginine and X₂ is lysine or arginine. In some embodiments, each enzymatic cleavage site comprises an amino acid sequence selected from RHKR (SEQ ID NO: 52), RHRR (SEQ ID NO: 53), RKKR (SEQ ID NO: 54), RKRR (SEQ ID NO: 55), RRKR (SEQ ID NO: 56), and RRRR (SEQ ID NO: 57). In some embodiments, each enzymatic cleavage site comprises an amino acid sequence of RRKR (SEQ ID NO: 56).

In some embodiments, the furin cleavage site comprises the amino acid sequence of any one of SEQ ID NOs: 52 to 57.

In some embodiments, the variant B/C junction and the variant C/A junction each comprise 4 to 10 amino acids selected from histidine, lysine, and arginine. In some embodiments, the variant B/C junction and the variant C/A junction each comprise 4 to 6 amino acids selected from histidine, lysine, and arginine. In some embodiments, the variant B/C junction and the variant C/A junction each comprise 4 amino acids selected from histidine, lysine, and arginine.

In some embodiments, the variant B/C junction comprises an amino acid sequence of XₙRX₁X₂R (SEQ ID NO: 64), wherein Xₙ is 0, 1, 2, 3, 4, 5, or 6 amino acids each chosen from histidine, lysine, or arginine, X₁ is histidine, lysine, or arginine, and X₂ is lysine or arginine. In some embodiments, the B/C and C/A junctions are the same. In some embodiments, the B/C and C/A junctions are different.

In some embodiments, the variant B/C junction comprises an amino acid sequence of SEQ ID NO: 52, SEQ ID NO: 53, SEQ ID NO: 54, SEQ ID NO: 55, SEQ ID NO: 56, SEQ ID NO: 57, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, or SEQ ID NO: 199.

In some embodiments, the variant B/C junction comprises an amino acid sequence of SEQ ID NO: 52, SEQ ID NO: 53, SEQ ID NO: 54, SEQ ID NO: 55, SEQ ID NO: 56, SEQ ID NO: 57, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, or SEQ ID NO: 136.

In some embodiments, the variant B/C junction comprises an amino acid sequence of RRKR (SEQ ID NO: 56).

In some embodiments, the variant C/A junction comprises an amino acid sequence of XₙRX₁X₂R (SEQ ID NO: 64), wherein Xₙ is 0, 1, 2, 3, 4, 5, or 6 amino acids each chosen from histidine, lysine, or arginine, X₁ is histidine, lysine, or arginine, and X₂ is lysine or arginine.

In some embodiments, the variant C/A junction comprises an amino acid sequence of SEQ ID NO: 52, SEQ ID NO: 53, SEQ ID NO: 54, SEQ ID NO: 55, SEQ ID NO: 56, SEQ ID NO: 57, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, or SEQ ID NO: 199.

In some embodiments, the variant C/A junction comprises an amino acid sequence of SEQ ID NO: 52, SEQ ID NO: 53, SEQ ID NO: 54, SEQ ID NO: 55, SEQ ID NO: 56, SEQ ID NO: 57, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, or SEQ ID NO: 136.

In some embodiments, the variant C/A junction comprises an amino acid sequence of RRKR (SEQ ID NO: 56).

In some embodiments, modification or substitution of the amino acids that comprise this amino acid sequence can dictate furin binding efficiency. In further embodiments, O-linked glycosylation in the furin binding pocket (P6-P1 and P1′-P2′ region) can alter the physical properties of this region and effect furin binding strength. In some embodiments, O-linked glycosylation modification is found on the Threonine (T) located in either position P6 or P5 of the 20 amino acid furin cleavage site. Steentoft, et al. (2013). In some embodiments, the presence of proline (P) in the P5 position is hypothesized to be overly rigid and disrupt the necessary structure or conformation needed for furin cleavage. In some embodiments, the presence of aspartic acid (D) in a P2′ position may dramatically increase the amount of overall negative charge in this region, which may in turn reduce binding.

Multiple exemplary variant preproinsulin constructs were designed, including those described in Table 4, below.

TABLE 4

| SEQ | N-Terminal Sig- | | | | | A-Chain |
|---|---|---|---|---|---|---|
| ID | nal Sequence | B-Chain | B/C | C-Peptide | | (SEQ ID |
| NO | (SEQ ID NO: 43) | (SEQ ID NO: 59) | Junction | (SEQ ID NO: 60) | C/A Junction | NO: 61) |
| 39 | MALWMRLLPLLAL | FVNQHLCGSHLVEALYLVC | RR | EAEDLQVGQVELGGGP | KR | GIVEQCCTSICSL |
| | LALWGPDPAAA | GERGFFYTPKT | (SEQ ID | GAGSLOPLALEGSLQ | (SEQ ID NO: 63) | YQLENYCN |
| | | | NO: 62) | | | |
| 3 | MALWMRLLPLLAL | FVNQHLCGSHLVEALYLVC | RHKR | EAEDLQVGQVELGGGP | RHKR | GIVEQCCTSICSL |
| | LALWGPDPAAA | GERGFFYTPKT | (SEQ ID | GAGSLQPLALEGSLQ | (SEQ ID NO: 52) | YQLENYCN |
| | | | NO: 52) | | | |
| 4 | MALWMRLLPLLAL | FVNQHLCGSHLVEALYLVC | RHKR | EAEDLQVGQVELGGGP | RHRR | GIVEQCCTSICSL |
| | LALWGPDPAAA | GERGFFYTPKT | (SEQ ID | GAGSLQPLALEGSLQ | (SEQ ID NO: 53) | YQLENYCN |
| | | | NO: 52) | | | |
| 5 | MALWMRLLPLLAL | FVNQHLCGSHLVEALYLVC | RHKR | EAEDLQVGQVELGGGP | RKKR | GIVEQCCTSICSL |
| | LALWGPDPAAA | GERGFFYTPKT | (SEQ ID | GAGSLQPLALEGSLQ | (SEQ ID NO: 54) | YQLENYCN |
| | | | NO: 52) | | | |
| 6 | MALWMRLLPLLAL | FVNQHLCGSHLVEALYLVC | RHKR | EAEDLQVGQVELGGGP | RKRR | GIVEQCCTSICSL |
| | LALWGPDPAAA | GERGFFYTPKT | (SEQ ID | GAGSLQPLALEGSLQ | (SEQ ID NO: 55) | YQLENYCN |
| | | | NO: 52) | | | |
| 7 | MALWMRLLPLLAL | FVNQHLCGSHLVEALYLVC | RHKR | EAEDLQVGQVELGGGP | RRKR | GIVEQCCTSICSL |
| | LALWGPDPAAA | GERGFFYTPKT | (SEQ ID | GAGSLQPLALEGSLQ | (SEQ ID NO: 56) | YQLENYCN |
| | | | NO: 52) | | | |
| 8 | MALWMRLLPLLAL | FVNQHLCGSHLVEALYLVC | RHKR | EAEDLQVGQVELGGGP | RRRR | GIVEQCCTSICSL |
| | LALWGPDPAAA | GERGFFYTPKT | (SEQ ID | GAGSLOPLALEGSLQ | (SEQ ID NO: 57) | YQLENYCN |
| | | | NO: 52) | | | |
| 9 | MALWMRLLPLLAL | FVNQHLCGSHLVEALYLVC | RHRR | EAEDLQVGQVELGGGP | RHKR | GIVEQCCTSICSL |
| | LALWGPDPAAA | GERGFFYTPKT | (SEQ ID | GAGSLQPLALEGSLQ | (SEQ ID NO: 52) | YQLENYCN |
| | | | NO: 53) | | | |
| 10 | MALWMRLLPLLAL | FVNQHLCGSHLVEALYLVC | RHRR | EAEDLQVGQVELGGGP | RHRR | GIVEQCCTSICSL |
| | LALWGPDPAAA | GERGFFYTPKT | (SEQ ID | GAGSLQPLALEGSLQ | (SEQ ID NO: 53) | YQLENYCN |
| | | | NO: 53) | | | |
| 11 | MALWMRLLPLLAL | FVNQHLCGSHLVEALYLVC | RHRR | EAEDLQVGQVELGGGP | RKKR | GIVEQCCTSICSL |
| | LALWGPDPAAA | GERGFFYTPKT | (SEQ ID | GAGSLQPLALEGSLQ | (SEQ ID NO: 54) | YQLENYCN |
| | | | NO: 53) | | | |
| 12 | MALWMRLLPLLAL | FVNQHLCGSHLVEALYLVC | RHRR | EAEDLQVGQVELGGGP | RKRR | GIVEQCCTSICSL |
| | LALWGPDPAAA | GERGFFYTPKT | (SEQ ID | GAGSLQPLALEGSLQ | (SEQ ID NO: 55) | YQLENYCN |
| | | | NO: 53) | | | |
| 13 | MALWMRLLPLLAL | FVNQHLCGSHLVEALYLVC | RHRR | EAEDLQVGQVELGGGP | RRKR | GIVEQCCTSICSL |
| | LALWGPDPAAA | GERGFFYTPKT | (SEQ ID | GAGSLQPLALEGSLQ | (SEQ ID NO: 56) | YQLENYCN |
| | | | NO: 53) | | | |
| 14 | MALWMRLLPLLAL | FVNQHLCGSHLVEALYLVC | RHRR | EAEDLQVGQVELGGGP | RRRR | GIVEQCCTSICSL |
| | LALWGPDPAAA | GERGFFYTPKT | (SEQ ID | GAGSLQPLALEGSLQ | (SEQ ID NO: 57) | YQLENYCN |
| | | | NO: 53) | | | |
| 15 | MALWMRLLPLLAL | FVNQHLCGSHLVEALYLVC | RKKR | EAEDLQVGQVELGGGP | RHKR | GIVEQCCTSICSL |
| | LALWGPDPAAA | GERGFFYTPKT | (SEQ ID | GAGSLQPLALEGSLQ | (SEQ ID NO: 52) | YQLENYCN |
| | | | NO: 54) | | | |
| 16 | MALWMRLLPLLAL | FVNQHLCGSHLVEALYLVC | RKKR | EAEDLQVGQVELGGGP | RHRR | GIVEQCCTSICSL |
| | LALWGPDPAAA | GERGFFYTPKT | (SEQ ID | GAGSLQPLALEGSLQ | (SEQ ID NO: 53) | YQLENYCN |
| | | | NO: 54) | | | |
| 17 | MALWMRLLPLLAL | FVNQHLCGSHLVEALYLVC | RKKR | EAEDLQVGQVELGGGP | RKKR | GIVEQCCTSICSL |
| | LALWGPDPAAA | GERGFFYTPKT | (SEQ ID | GAGSLQPLALEGSLQ | (SEQ ID NO: 54) | YQLENYCN |
| | | | NO: 54) | | | |
| 18 | MALWMRLLPLLAL | FVNQHLCGSHLVEALYLVC | RKKR | EAEDLQVGQVELGGGP | RKRR | GIVEQCCTSICSL |
| | LALWGPDPAAA | GERGFFYTPKT | (SEQ ID | GAGSLQPLALEGSLQ | (SEQ ID NO: 55) | YQLENYCN |
| | | | NO: 54) | | | |
| 19 | MALWMRLLPLLAL | FVNQHLCGSHLVEALYLVC | RKKR | EAEDLQVGQVELGGGP | RRKR | GIVEQCCTSICSL |
| | LALWGPDPAAA | GERGFFYTPKT | (SEQ ID | GAGSLQPLALEGSLQ | (SEQ ID NO: 56) | YQLENYCN |
| | | | NO: 54) | | | |
| 20 | MALWMRLLPLLAL | FVNQHLCGSHLVEALYLVC | RKKR | EAEDLQVGQVELGGGP | RRRR | GIVEQCCTSICSL |
| | LALWGPDPAAA | GERGFFYTPKT | (SEQ ID | GAGSLQPLALEGSLQ | (SEQ ID NO: 57) | YQLENYCN |
| | | | NO: 54) | | | |
| 21 | MALWMRLLPLLAL | FVNQHLCGSHLVEALYLVC | RKRR | EAEDLQVGQVELGGGP | RHKR | GIVEQCCTSICSL |
| | LALWGPDPAAA | GERGFFYTPKT | (SEQ ID | GAGSLQPLALEGSLQ | (SEQ ID NO: 52) | YQLENYCN |
| | | | NO: 55) | | | |
| 22 | MALWMRLLPLLAL | FVNQHLCGSHLVEALYLVC | RKRR | EAEDLQVGQVELGGGP | RHRR | GIVEQCCTSICSL |
| | LALWGPDPAAA | GERGFFYTPKT | (SEQ ID | GAGSLQPLALEGSLQ | (SEQ ID NO: 53) | YQLENYCN |
| | | | NO: 55) | | | |
| 23 | MALWMRLLPLLAL | FVNQHLCGSHLVEALYLVC | RKRR | EAEDLQVGQVELGGGP | RKKR | GIVEQCCTSICSL |
| | LALWGPDPAAA | GERGFFYTPKT | (SEQ ID | GAGSLQPLALEGSLQ | (SEQ ID NO: 54) | YQLENYCN |
| | | | NO: 55) | | | |
| 24 | MALWMRLLPLLAL | FVNQHLCGSHLVEALYLVC | RKRR | EAEDLQVGQVELGGGP | RKRR | GIVEQCCTSICSL |
| | LALWGPDPAAA | GERGFFYTPKT | (SEQ ID | GAGSLQPLALEGSLQ | (SEQ ID NO: 55) | YQLENYCN |
| | | | NO: 55) | | | |
| 25 | MALWMRLLPLLAL | FVNQHLCGSHLVEALYLVC | RKRR | EAEDLQVGQVELGGGP | RRKR | GIVEQCCTSICSL |
| | LALWGPDPAAA | GERGFFYTPKT | (SEQ ID | GAGSLQPLALEGSLQ | (SEQ ID NO: 56) | YQLENYCN |
| | | | NO: 55) | | | |
| 26 | MALWMRLLPLLAL | FVNQHLCGSHLVEALYLVC | RKRR | EAEDLQVGQVELGGGP | RRRR | GIVEQCCTSICSL |
| | LALWGPDPAAA | GERGFFYTPKT | (SEQ ID | GAGSLQPLALEGSLQ | (SEQ ID NO: 57) | YQLENYCN |
| | | | NO: 55) | | | |
| 27 | MALWMRLLPLLAL | FVNQHLCGSHLVEALYLVC | RRKR | EAEDLQVGQVELGGGP | RHKR | GIVEQCCTSICSL |
| | LALWGPDPAAA | GERGFFYTPKT | (SEQ ID | GAGSLQPLALEGSLQ | (SEQ ID NO: 52) | YQLENYCN |
| | | | NO: 56) | | | |
| 28 | MALWMRLLPLLAL | FVNQHLCGSHLVEALYLVC | RRKR | EAEDLQVGQVELGGGP | RHRR | GIVEQCCTSICSL |
| | LALWGPDPAAA | GERGFFYTPKT | (SEQ ID | GAGSLQPLALEGSLQ | (SEQ ID NO: 53) | YQLENYCN |
| | | | NO: 56) | | | |
| 29 | MALWMRLLPLLAL | FVNQHLCGSHLVEALYLVC | RRKR | EAEDLQVGQVELGGGP | RKKR | GIVEQCCTSICSL |
| | LALWGPDPAAA | GERGFFYTPKT | (SEQ ID | GAGSLOPLALEGSLQ | (SEQ ID NO: 54) | YQLENYCN |
| | | | NO: 56) | | | |
| 30 | MALWMRLLPLLAL | FVNQHLCGSHLVEALYLVC | RRKR | EAEDLQVGQVELGGGP | RKRR | GIVEQCCTSICSL |
| | LALWGPDPAAA | GERGFFYTPKT | (SEQ ID | GAGSLQPLALEGSLQ | (SEQ ID NO: 55) | YQLENYCN |
| | | | NO: 56) | | | |
| 31 | MALWMRLLPLLAL | FVNQHLCGSHLVEALYLVC | RRKR | EAEDLQVGQVELGGGP | RRKR | GIVEQCCTSICSL |
| | LALWGPDPAAA | GERGFFYTPKT | (SEQ ID | GAGSLQPLALEGSLQ | (SEQ ID NO: 56) | YQLENYCN |
| | | | NO: 56) | | | |
| 32 | MALWMRLLPLLAL | FVNQHLCGSHLVEALYLVC | RRKR | EAEDLQVGQVELGGGP | RRRR | GIVEQCCTSICSL |
| | LALWGPDPAAA | GERGFFYTPKT | (SEQ ID | GAGSLQPLALEGSLQ | (SEQ ID NO: 57) | YQLENYCN |
| | | | NO: 56) | | | |
| 33 | MALWMRLLPLLAL | FVNQHLCGSHLVEALYLVC | RRRR | EAEDLQVGQVELGGGP | RHKR | GIVEQCCTSICSL |
| | LALWGPDPAAA | GERGFFYTPKT | (SEQ ID | GAGSLQPLALEGSLQ | (SEQ ID NO: 52) | YQLENYCN |
| | | | NO: 57) | | | |
| 34 | MALWMRLLPLLAL | FVNQHLCGSHLVEALYLVC | RRRR | EAEDLQVGQVELGGGP | RHRR | GIVEQCCTSICSL |
| | LALWGPDPAAA | GERGFFYTPKT | (SEQ ID | GAGSLQPLALEGSLQ | (SEQ ID NO: 53) | YQLENYCN |
| | | | NO: 57) | | | |
| 35 | MALWMRLLPLLAL | FVNQHLCGSHLVEALYLVC | RRRR | EAEDLQVGQVELGGGP | RKKR | GIVEQCCTSICSL |
| | LALWGPDPAAA | GERGFFYTPKT | (SEQ ID | GAGSLQPLALEGSLQ | (SEQ ID NO: 54) | YQLENYCN |
| | | | NO: 57) | | | |
| 36 | MALWMRLLPLLAL | FVNQHLCGSHLVEALYLVC | RRRR | EAEDLQVGQVELGGGP | RKRR | GIVEQCCTSICSL |
| | LALWGPDPAAA | GERGFFYTPKT | (SEQ ID | GAGSLQPLALEGSLQ | (SEQ ID NO: 55) | YQLENYCN |
| | | | NO: 57) | | | |
| 37 | MALWMRLLPLLAL | FVNQHLCGSHLVEALYLVC | RRRR | EAEDLQVGQVELGGGP | RRKR | GIVEQCCTSICSL |
| | LALWGPDPAAA | GERGFFYTPKT | (SEQ ID | GAGSLOPLALEGSLQ | (SEQ ID NO: 56) | YQLENYCN |
| | | | NO: 57) | | | |
| 38 | MALWMRLLPLLAL | FVNQHLCGSHLVEALYLVC | RRRR | EAEDLQVGQVELGGGP | RRRR | GIVEQCCTSICSL |
| | LALWGPDPAAA | GERGFFYTPKT | (SEQ ID | GAGSLOPLALEGSLQ | (SEQ ID NO: 57) | YQLENYCN |
| | | | NO: 57) | | | |
| SEQ | N-Terminal Signal | | | | | |
| ID | Sequence | B-Chain | B/C | C-Peptide | | A-Chain |
| NO | (SEQ ID NO: 65) | (SEQ ID NO: 66) | Junction | (SEQ ID NO: 67) | C/A Junction | (SEQ ID NO: 68) |
| 46 | MALWMRLLPLLAL | FVNQHLCGSHLVEALYLVC | RR | EVEDLQVRDVELAGAP | KR | GIVEQCCTSICSL |
| | LALWAPAPTRA | GERGFFYTPKA | (SEQ ID | GEGGLQPLALEGALQ | (SEQ ID NO: 63) | YQLENYCN |
| | | | NO: 62) | | | |
| 47 | MALWMRLLPLLAL | FVNQHLCGSHLVEALYLVC | RRKR | EVEDLQVRDVELAGAP | RRKR | GIVEQCCTSICSL |
| | LALWAPAPTRA | GERGFFYTPKA | (SEQ ID | GEGGLQPLALEGALQ | (SEQ ID NO: 56) | YQLENYCN |
| | | | NO: 56) | | | |
| SEQ | N-Terminal Signal | | | | | |
| ID | Sequences | B-Chain | B/C | C-Peptide | | A-Chain |
| NO | (SEQ ID NO: 69) | (SEQ ID NO: 66) | Junction | (SEQ ID NO: 70) | C/A Junction | (SEQ ID NO: 71) |
| 49 | MAPWTRLLPLLAL | FVNQHLCGSHLVEALYLVC | RR | EAEDLQGKDAELGEAP | KR | GIVEQCCASVCSL |
| | LSLWIPAPTRA | GERGFFYTPKA | (SEQ ID | GAGGLQPSALEAPLQ | (SEQ ID NO: 63) | YQLEHYCN |
| | | | NO: 62) | | | |
| 50 | MAPWTRLLPLLAL | FVNQHLCGSHLVEALYLVC | RRKR | EAEDLQGKDAELGEAP | RRKR | GIVEQCCASVCSL |
| | LSLWIPAPTRA | GERGFFYTPKA | (SEQ ID | GAGGLQPSALEAPLQ | (SEQ ID NO: 56) | YQLEHYCN |
| | | | NO: 56) | | | |

### C. Host Cells, Vectors, And Packaging Cell Lines

The present disclosure provides host cells transduced with the constructs described herein. As used herein, the term “host cell” is used to refer to any prokaryotic or eukaryotic cell that contains the constructs of the present invention. This term also includes cells that have been genetically engineered to integrate the constructs of the present invention into the genome of the host cell.

As used herein, the term “nucleic acid construct” refers to an artificially designed nucleic acid molecule. Nucleic acid constructs may be part of a vector that is used, for example, to transform a cell. When referring to a nucleic acid molecule alone (as opposed to a viral particle), the term “vector” is used herein.

As used herein, the term “promoter” refers to a DNA sequence capable of controlling the expression of a coding sequence or functional RNA. In general, a coding sequence is located 3′ to a promoter sequence. Promoters may be derived in their entirety from a native gene or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters that cause a gene to be expressed in most cell types at most times are commonly referred to as “constitutive promoters”. Promoters that allow the selective expression of a gene in most cell types are referred to as “inducible promoters” or “tissue specific promoters”.

As used herein, the term “liver-specific promoter” is used to refer to a promoter that predominantly, if not only, drives expression of a functionally linked gene in liver cells (i.e., hepatocytes). A liver-specific promoter is used with the constructs of the present invention to ensure that production of insulin is restricted only to liver cells when the constructs are utilized in a gene therapy. Any constitutively active liver-specific promoter that is capable of driving sustained, moderate- to high-level transcription can be used in the constructs of the present invention. An example of such a promoter is alpha 1-antitrypsin inhibitor (Hafenrichter et al. (1994)). In some embodiments, the liver-specific promoter is an albumin promoter. For example, in some embodiments, the albumin promoter is the rat albumin promoter (which was produced as described in Alam, et al. (2002); Heard et al. (1987)).

As used herein, the term “translational enhancer” is used to refer to a sequence that promotes translation of a functionally linked gene. In some embodiments, the translational enhancer is a vascular endothelial growth factor (VEGF) translational enhancer. The VEGF translational enhancer acts as a ribosomal entry site; allowing it to increase the effectiveness of the translation process. Thus, its presence causes a larger amount of insulin protein production from a given amount of insulin mRNA.

As used herein, “vector” includes any genetic element, including, but not limited to, a plasmid, phage, transposon, cosmid, chromosome, artificial chromosome, minichromosome, expression vector, virus, virion, mRNA, etc., which is capable of replication when associated with the proper control elements and which can transfer nucleic acid molecules to cells. The term includes cloning and expression vectors, as well as viral vectors. The term includes the vector as a self-replicating nucleic acid structure that can be packaged into viral particles and can be expressed in dividing and non-dividing cells either extra chromosomally or integrated into the host cell genome. Certain vectors are capable of directing the expression of nucleic acids to which they are operatively linked. Such vectors are referred to as “expression vectors.”

As used herein, the term “virus particle” is used to refer to a virion consisting of nucleic acid surrounded by a protective coat of protein called a capsid. The term “viral vector” is used to describe a virus particle used to deliver genetic material (e.g., the constructs of the present invention) into cells. The shorthand “AAV vector” or “AAV8 vector” is commonly used to refer to a viral vector in the art.

In some embodiments, the invention comprises a vector. In some embodiments, a vector comprises a nucleic acid described herein. In some embodiments, the vector is a viral vector. In some embodiments, the vector is an adeno-associated virus (AAV) vector, an adenovirus vector, a retrovirus vector, a herpesvirus vector, or a pox virus vector. In some embodiments, the vector is an adeno-associated virus (AAV) vector. In some embodiments, the vector is a self-complementary adeno-associated virus (scAAV) vector. In some embodiments, the vector is an adeno-associated virus (AAV) vector having a capsid serotype selected from the group consisting of AAV1, AAV2, AAV3, AAV4, AAV5, AAV6, AAV7, AAV8, AAV9, AAV10, AAV11, AAV12, AAV13, and any variant thereof. In some embodiments, the AAV vector has a capsid serotype of AAV8. In some embodiments, the vector is a synthetic mRNA. In some embodiments, the vector is a self-replicating RNA.

In some embodiments, the vector comprises a nucleic acid molecule comprising a nucleic acid sequence of SEQ ID NO: 76 or SEQ ID NO: 77.

As used herein, the term “packaging cell line” is used to refer to a cell line that provides all the proteins necessary for virus production and maturation. Suitable packaging cell lines for use with the present disclosure include, without limitation, HEK 293T cells and HEK 293 cell variants.

The present disclosure also provides packaging cell lines for producing the virus particles described herein. The packaging cell line should be selected with the method of viral production in mind. For example, cells that have strong adhesion properties should be selected for growth in culture plates, whereas cells lacking adhesion properties should be selected for growth in suspension culture.

### D. Gene Therapy

The field of gene therapy has experienced recent successes in the treatment of single-gene disorders. Gene therapy generally involves using a carrier (e.g., a virus, liposome, etc.) to deliver a therapeutic gene (or genes) to target cells for expression of the encoded therapeutic agent(s) for the treatment and/or prevention of a disease or condition. The gene therapy of the present disclosure is designed to deliver an insulin-encoding nucleic acid molecule to a subject. In some embodiments, these gene therapies may be delivered to the liver of a subject and enable the hepatocytes (i.e., liver cells) to synthesize and secrete insulin, for example in response to changing glucose levels. A single intravenous treatment of these gene therapies may be adequate to control hyperglycemia in mice for the duration of a substantial portion of their lives. Thus, these gene therapies may provide long-term benefits to patients with type I diabetes.

In some embodiments, the nucleic acid construct is introduced to the diabetic patients in muscle or myoblast cells, wherein the cells are treated in vitro or in vivo to incorporate therein the DNA fragment, which results in the cells expressing the therapeutically effective amount of insulin in vivo in the diabetic patient. In some embodiments, the nucleic acid construct can be introduced into the cell by standard gene transfection methods, e.g., calcium phosphate precipitation or by a viral vector, e.g., adenoviral vector. Examples of suitable vectors include viral vectors (e.g. retroviral vectors, adenoviral vectors, adeno-associated viral vectors, sindbis viral vectors, and herpes viral vector), plasmids, cosmids, and yeast artificial chromosomes. In some embodiments, the nucleic acid construct can be introduced by a recombinant adeno-associated virus (AAV). In some embodiments, the nucleic acid construct can be introduced by an AAV serotype 8 (AAV8). The cells may be introduced to the host by standard transplantation techniques, or in a neoorgan, or in a matrix, e.g., microencapsulated in sodium alginate, or contained within an immunoprotected cell factory. The nucleic acid construct segment can also be introduced as infectious particles, e.g., DNA-ligand conjugates, calcium phosphate precipitates, and liposomes. In some embodiments, gene delivery comprises transfer of a synthetic mRNA. In some embodiments, gene delivery comprises transfer of self-replicating RNA.

In some embodiments, one or more of the vectors, or all of the vectors, may be DNA vectors. In some embodiments, one or more of the vectors, or all of the vectors, may be RNA vectors. In some embodiments, one or more of the vectors, or all of the vectors, may be circular. In other embodiments, one or more of the vectors, or all of the vectors, may be linear. In some embodiments, one or more of the vectors, or all of the vectors, may be enclosed in a lipid nanoparticle, liposome, non-lipid nanoparticle, or viral capsid.

Non-limiting exemplary viral vectors include adeno-associated virus (AAV) vector, lentivirus vectors, adenovirus vectors, helper-dependent adenoviral vectors (HDAd), herpes simplex virus (HSV-1) vectors, bacteriophage T4, baculovirus vectors, pox virus vectors, and retrovirus vectors. In some embodiments, the viral vector may be an AAV vector. In other embodiments, the viral vector may a lentivirus vector. In some embodiments, the lentivirus may be non-integrating. In some embodiments, the viral vector may be an adenovirus vector. In yet other embodiments, the viral vector may be an HSV-1 vector. In some embodiments, the HSV-1-based vector is helper dependent, and in other embodiments it is helper independent. In additional embodiments, the viral vector may be bacteriophage T4. In further embodiments, the viral vector may be a baculovirus vector. In yet further embodiments, the viral vector may be a retrovirus vector.

Adeno-associated virus (AAV) is a preferred gene therapy vector because of its proven gene delivery effect, low immunogenicity, and apparent lack of pathogenicity. In various embodiments, an AAV vector may comprise a nucleic acid molecule enclosed in an AAV viral capsid. The encapsulated nucleic acid molecule may comprise AAV inverted terminal repeats (ITRs) positioned at each termini. AAV ITRs may be derived from any number of AAV serotypes, including AAV2. AAV vector genomes may comprise single-stranded or double-stranded DNA. AAV ITRs can form hairpin structures and are involved in AAV proviral integration and vector packaging. An AAV vector may comprise a capsid protein from any one of the AAV serotypes, including, but not limited to AAV1, AAV2, AAV3, AAV4, AAV5, AAV6, AAV7, AAV8, AAV9, AAV10, AAV11, AAV12, AAV13, and variant capsids based on any serotype modified to target a specific cell type. In some embodiments, an AAV vector comprises a nucleic acid molecule with AAV2 ITRs and enclosed in an AAV8 viral capsid.

AAV vectors may be prepared using any one of the number of methods available to those of ordinary skill in the art. Hermonat, et al. (1984); Liu, et al. (2001); Grimm, et al. (1998); Neyns, et al. (2001); Cecchini, et al. (2011).

In some embodiments, a vector comprises a nucleic acid molecule encoding a transgene that is operatively linked to a promoter. The phrases “operatively positioned,” “operatively linked,” “under control,” or “under transcriptional control” means that a promoter is in the correct location and orientation in relation to the nucleic acid molecule to control RNA polymerase initiation and expression of the transgene.

As used herein, the term “promoter enhancer” is used to refer to a sequence that promotes transcription of a functionally linked gene by enhancing promoter function. Any promoter enhancer that enhances the activity of the liver-specific promoter included in the construct may be used with the present invention. In some embodiments, the promoter enhancer is an alpha-fetoprotein enhancer. In these embodiments, the alpha-fetoprotein enhancer increases the effectiveness of albumin promoter and increases the binding of RNA polymerase complex, thereby causing an increase in mRNA production, and ultimately leading to an increase in protein production. In wildtype, endogenous transcription factors present in liver cells interact with the alpha-fetoprotein enhancer region, activating the alpha-fetoprotein promoter. It should be noted, in the fully developed liver, the alpha-fetoprotein enhancer can also be repressed. Accordingly, in some embodiments, the region associated with this repression is not included in the alpha-fetoprotein enhancer sequence, allowing enhancer activity to persist in fully developed liver cells.

As used herein, the term “glucose inducible regulatory element” or “GIRE” refers to a glucose-responsive DNA motif found in the promoter region of glucose-inducible genes, such as LPK, S14, fatty acid synthase, and acetyl-CoA carboxylase. In some embodiments, the GIRE is composed of two tandem repeats of 5′-CACGTG (known as E boxes), separated by five base pairs. When a specific transcription factor, Carbohydrate Response Element-binding Protein (ChREBP), recognizes these E box sequences, it results in glucose-responsive control of gene transcription.

In some embodiments, the invention further comprises a promoter. In some embodiments, the nucleic acid molecule further comprises a promoter operatively linked to the nucleic acid sequence encoding the variant preproinsulin polypeptide. In some embodiments, the promoter is a constitutive promoter. In some embodiments, the promoter is a regulated promoter. In some embodiments, the promoter is an albumin promoter. In some embodiments, the nucleic acid molecule further comprises at least one GIRE element.

In some embodiments, the vector may be capable of driving expression of a coding sequence of a transgene disclosed herein, in a host cell, either in vivo, ex vivo, or in vitro. In some embodiments, the host cell is a eukaryotic cell, such as, e.g., a yeast, plant, insect, or mammalian cell. In some embodiments, the host cell is a mammalian cell. In some embodiments, the host cell is a rodent cell. In some embodiments, the host cell is a human cell. In some embodiments, the host cell is a canine cell. In some embodiments, the host cell is a feline cell. In some embodiments, the host cell is a smooth muscle cell, a cardiac muscle cell, a fibroblast cell, a liver hepatocyte, or an immune cell (e.g., a monocyte, a macrophage, a dendritic cell, a T-cell, or a B-cell). Breuer et al. 2020 demonstrates that AAV vectors can mediate transduction of both intracellular and transmembrane proteins in multiple immune cell types, including CD4+ T cells, CD8+ T cells, B cells, macrophages, and dendritic cells in mice. In some embodiments, the cells may be used to manufacture insulin for medical use.

### E. Cell Replacement Therapy

Hepatocytes have long been a preferred target as sources of surrogate p cells. They are long-lived and robust protein factories that are capable of the production and secretion of therapeutic proteins. Additionally, hepatocytes receive an extensive blood supply and are accessible to blood-borne particles, such as viruses. Moreover, they express glucose-sensing molecules nearly identical to those in the pancreas (i.e., GLUT2, glucokinase), and thus have the intrinsic ability to respond to changes in blood glucose concentration.

This disclosure also provides a cultured host cell comprising a nucleic acid molecule described herein, the variant preproinsulin polypeptide described herein, or the vector described herein.

In some embodiments, the invention includes a method of treating a subject with diabetes comprising administering to the subject the cultured host cell as described herein.

In some embodiments, the cultured host cell, is administered to the subject via intravenous injection, arterial injection, intramuscular injection, intradermal injection, intraperitoneal injection, and/or subcutaneous injection.

### F. Exemplary Pharmaceutical Compositions and Uses

“Pharmaceutical composition” refers to a preparation which is in such form as to permit administration of a therapeutic agent and other component(s) contained therein to a subject and does not contain components that are unacceptably toxic to a subject.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. A pharmaceutical composition may be in the form of solid, semisolid, liquid, cream, gel, capsule, or patch. The pharmaceutical composition may be in a form that allows for slow release or delayed release of a therapeutic agent.

Pharmaceutically acceptable carrier encompasses any of the agents approved by a regulatory agency of the US Federal government or listed in the US Pharmacopeia for use in animals, including humans.

Examples of pharmaceutically acceptable carriers include alumina; aluminum stearate; lecithin; serum proteins, such as human serum albumin, canine or other animal albumin; buffers such as phosphate, citrate, tromethamine or HEPES buffers; glycine; sorbic acid; potassium sorbate; partial glyceride mixtures of saturated vegetable fatty acids; water; salts or electrolytes, such as protamine sulfate, disodium hydrogen phosphate, potassium hydrogen phosphate, sodium chloride, zinc salts, colloidal silica, or magnesium trisilicate; polyvinyl pyrrolidone, cellulose-based substances; polyethylene glycol; sucrose; mannitol; or amino acids including, but not limited to, arginine. The carrier may be Zolgensma buffer.

“Therapeutic agent,” as used herein, refers to an agent used for the treatment or prevention of a disease, condition, or disorder. A therapeutic agent may include a nucleic acid molecule, polypeptide, vector, viral vector, and/or cell disclosed herein.

The pH of a pharmaceutical composition is typically in the range of from about pH 6 to pH 8 when administered, for example about 6, about 6.2, about 6.4, about 6.6, about 6.8, about 7, about 7.2. Pharmaceutical compositions may be sterilized if they are to be used for therapeutic purposes. Sterility can be achieved by any of several means known in the art, including by filtration through sterile filtration membranes (e.g., 0.2 micron membranes). Sterility may be maintained with or without anti-bacterial agents.

One aspect of this disclosure provides a method of treating a subject with diabetes comprising administering to the subject a nucleic acid described herein, a variant preproinsulin polypeptide described herein, a vector described herein, a viral vector described herein, a cultured host cell described herein, or a pharmaceutical composition described herein.

This disclosure further provides a method of producing insulin in a cell, the method comprising transducing, transfecting, or transforming the cell with a nucleic acid molecule described herein or a vector described herein.

In some embodiments, the invention is a method of treating a subject with diabetes comprising administering to the subject a nucleic acid, a variant preproinsulin polypeptide, a vector, a viral vector, a cultured cell, and/or a pharmaceutical composition described herein, wherein the variant preproinsulin polypeptide, the vector, the viral vector, the cultured host cell, and/or the pharmaceutical composition is administered to the subject via intravenous injection, arterial injection, intramuscular injection, intradermal injection, intraperitoneal injection, and/or subcutaneous injection.

In some embodiments, the subject is a human, a dog, or a cat. In some embodiments, the subject is a human subject. In some embodiments, the diabetes is Type 1 diabetes.

In some embodiments, the vector is administered to the subject at a dose of about 1×10¹⁰ vector genomes per kilogram, about 1×10¹¹ vector genomes per kilogram, about 1×10¹² vector genomes per kilogram, about 1×10¹³ vector genomes per kilogram, about 1×10¹⁰ to about 1×10¹³ vector genomes per kilogram, about 1×10¹⁰ to about 1×10¹² vector genomes per kilogram, about 1×10¹¹ to about 1×10¹² vector genomes per kilogram, or about 1×10¹⁰ to about 1×10¹¹ vector genomes per kilogram.

### EXAMPLES

### Example 1. Analysis of Limitations on Detecting 1994 Groskreutz and 1992 Yanagita Preproinsulin Design Products

Commercially available ELISA assays that are capable of detecting the fully and/or partially processed protein products generated from processing rat and/or human proinsulin proteins were considered.

### A. 1994 Groskreutz-Rat

The protein products produced using the 1994 Groskreutz-Rat design (a construct encoding rat preproinsulin mutated to contain two, four-amino acid furin cleavage sites-one spanning the B-chain and B/C junction and a second spanning the C-peptide and C/A junction; SEQ ID NO: 42) were characterized by ELISA. The following ELISA kits were capable of detecting the partially and fully processed proinsulin protein products produced from the 1994 Groskreutz-Rat construct: Mercodia Ultrasensitive Rat Insulin ELISA (Catalog No. 10-1251-01), Promega Lumit Insulin Immunoassay, and Mercodia Rat C-peptide ELISA (Catalog No. 10-1172-01). Additional ELISA kits tested included the Mercodia Ultrasensitive Insulin ELISA (Catalog No. 10-1132-01) and the Mercodia C-peptide ELISA (Catalog No. 10-1136-01), but as expected these human specific ELISAs were unable to detect the Groskreutz-Rat products as the antibodies in those kits are specific for human insulin and human C-peptide and do no cross react with the corresponding rat peptides.

### B. 1994 Groskreutz-Human

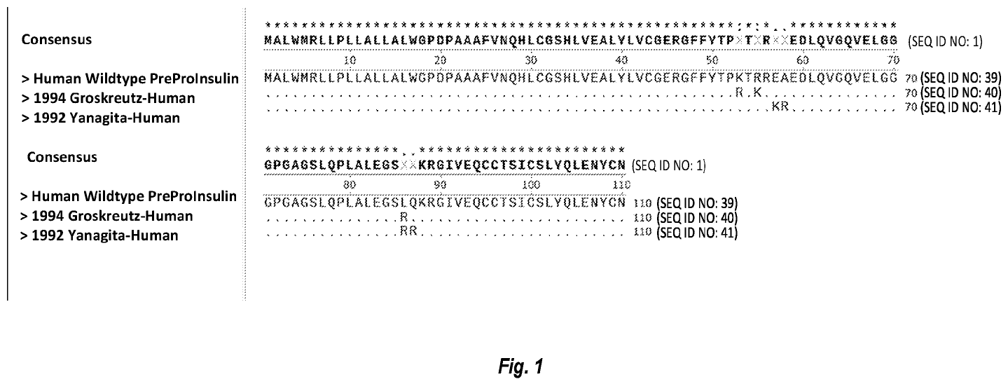

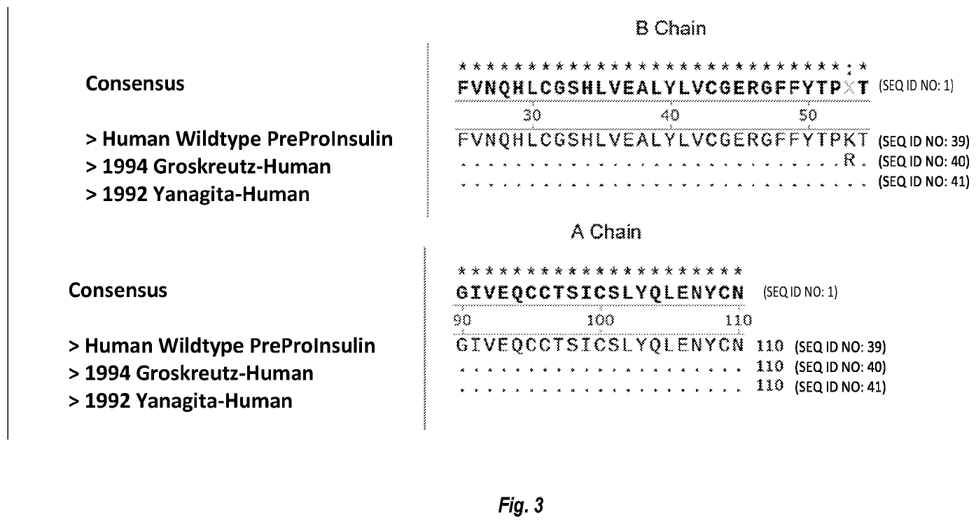

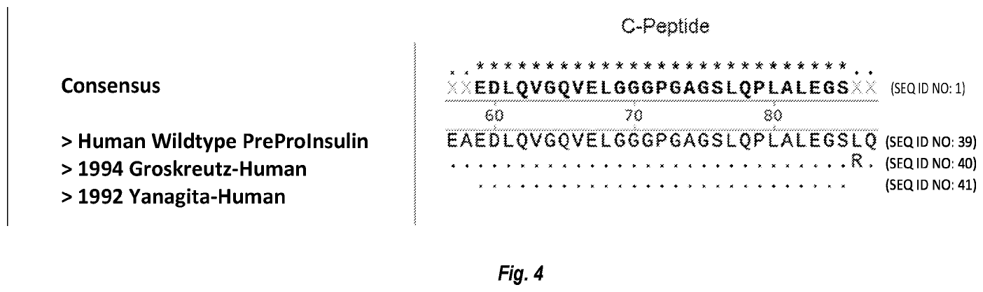

The 1994 Groskreutz-Human construct has the same style of amino acid substitutions as the 1994 Groskreutz-Rat construct (see above), but is based on the wild-type human preproinsulin sequence. FIG. 1 provides an alignment of the human wildtype preproinsulin sequences as compared to the 1994 Groskreutz-Human. As shown, there are three amino acid changes between wildtype human preproinsulin and 1994 Groskreutz-Human preproinsulin. Furthermore, following processing, the mature insulin generated by 1994 Groskreutz-Human contains a single conservative amino acid change (K to R) at the penultimate amino acid of the B-chain and a single nonconservative amino acid change (L to R) at the penultimate amino acid of the C-peptide. See FIGS. 3 and 4 (showing 1994 Groskreutz-Human B-chain compared to human wildtype B-chain and 1994 Groskreutz-Human C-peptide comparted to human wildtype C-peptide, respectively).

The processed proinsulin protein products produced from the 1994 Groskreutz-Human (m-human insulin; SEQ ID NO: 40) construct were also characterized by ELISA. Commercially available ELISA assays that are capable of detecting the fully and/or partially processed protein products generated from processing rat and/or human proinsulin proteins were considered. For the 1994 Groskreutz-Human construct assays, the following ELISA kits detected insulin products: Mercodia Ultrasensitive Rat Insulin ELISA (Catalog No. 10-1251-01) and Promega Lumit Insulin Immunoassay. The Ultrasensitive Rat Insulin ELISA kit is a pan-insulin kit, meaning it recognizes insulin from many different species, and also recognizes partially processed forms of insulin. The Promega Lumit Insulin Immunoassay kit is also a pan-insulin kit.

Unexpectedly, the following human insulin and C-peptide specific kits did not detect protein products of 1994 Groskreutz-Human: Mercodia Ultrasensitive Insulin ELISA (Catalog no. 10-1132-01) and Mercodia Ultrasensitive C-peptide ELISA (Catalog no. 10-1141-01). As noted above, the penultimate amino acids are mutated for both the human C-peptides and human insulin B-chains produced by the 1994 Groskreutz-Human construct. See FIG. 3 and FIG. 4, showing modification of the products of 1994 Groskreutz-Human compared to the products of human wildtype preproinsulin (SEQ ID NO: 39). The Mercodia Ultrasensitive Insulin and the Mercodia Ultrasensitive C-peptide ELISAs were unable to detect the products of 1994 Groskreutz-Human because the mutations present in the B-chain and C-peptide interfere with antibody binding using the corresponding kit (information courtesy of Mercodia).

Upon further analysis of the 1994 Groskreutz-Human and 1994 Groskreutz-Rat constructs, it became clear that the generation of variant C-peptides and variant B-chains was problematic because commercially available kits specific for human C-peptide and human insulin were unable to detect and monitor the products produced.

### C. 1992 Yanagita-Human

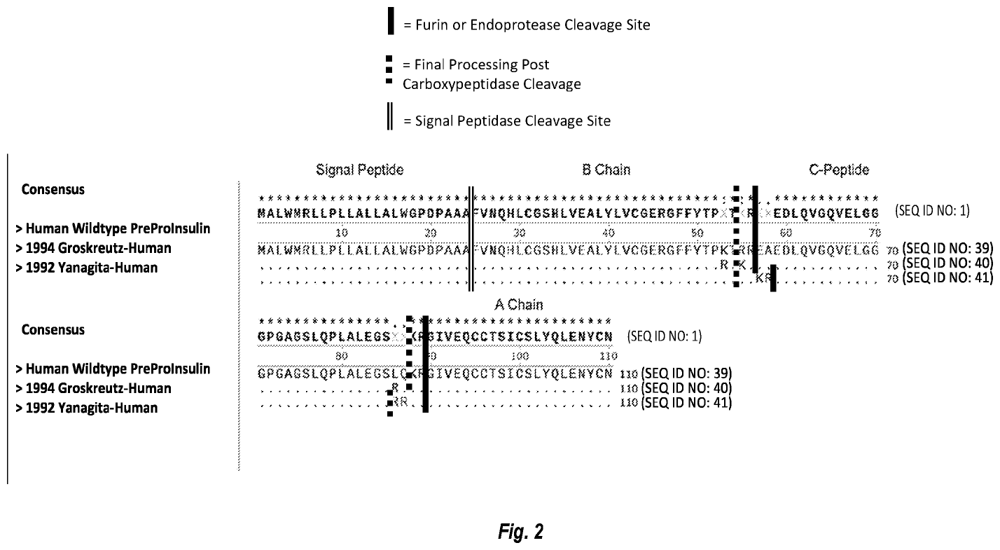

Another common design for furin cleavable insulin is the 1992 Yanagita design. As detailed in FIG. 1, there are four amino acid substitutions between the wildtype human proinsulin sequence and 1992 Yanagita-Human sequence. As shown in FIG. 2, for the 1992 Yanagita-Human construct, the changes made to create the first of the two artificial furin cleavage sites, in the area of the B/C junction, results in a shift of cleavage by two amino acids compared to the endoprotease cleavage site of wildtype human proinsulin. This change results in two more amino acids being removed by carboxypeptidase from the 1992 Yanagita-Human B-chain compared to the wildtype human sequence to generate a mature B-chain. This difference also results in a truncation of the amino-terminus of the C-peptides generated by the 1992 Yanagita-Human construct by two amino acids compared to wildtype C-peptide. FIG. 2 also shows how the changes made to create the second of the two artificial furin cleavage sites at the C/A junction in the 1992 Yanagita-Human construct results in an additional two additional amino acids being removed from the C-peptide at the carboxy-terminus by carboxypeptidase compared to the wildtype human C-peptide. As a result, the mature insulin generated by 1992 Yanagita-Human is identical to mature wildtype human insulin, but the mature C-peptide generated by processing 1992 Yanagita-Human is four amino acids smaller than the mature C-peptide generated by processing wildtype human insulin (but otherwise has the identical amino acid sequence). Unlike 1994 Groskreutz-Human, a fully processed 1992 Yanagita-Human proinsulin protein generates wildtype insulin protein having a wildtype B-chain sequence and wild-type A-chain sequence.

The processed insulin protein products produced from the 1992 Yanagita-Human (SEQ ID NO: 41) were also characterized by ELISA. For the 1992 Yanagita-Human construct assays, the following ELISA kits detected insulin products: the Mercodia Ultrasensitive Rat Insulin ELISA (Catalog No. 10-1251-01) and the Promega Lumit Insulin Immunoassay. Again, this result was expected as both the Mercodia Ultrasensitive Rat Insulin ELISA kit and the Promega Lumit Insulin Immunoassay kit are pan-insulin ELISA kits. Additionally, as expected, the Mercodia Human Ultrasensitive Insulin ELISA (Catalog no. 10-1132-01) was able to recognize the mature wildtype human insulin generated by the 1992 Yanagita-Human design. On the other hand, the Mercodia Human Ultrasensitive C-peptide ELISA (Catalog no. 10-1141-01) was unable to detect the mature but truncated C-peptide produced by the 1992 Yanagita-Human design. This appears to be due to one of the C-peptide epitopes recognized by the Mercodia Human Ultrasensitive C-peptide ELISA being absent in the truncated C-peptide generated by the 1992 Yanagita-Human design (information courtesy of Mercodia).

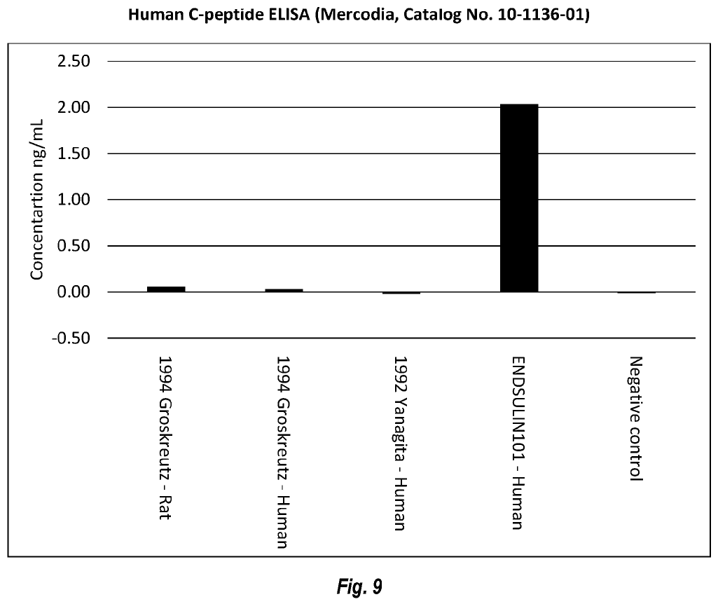

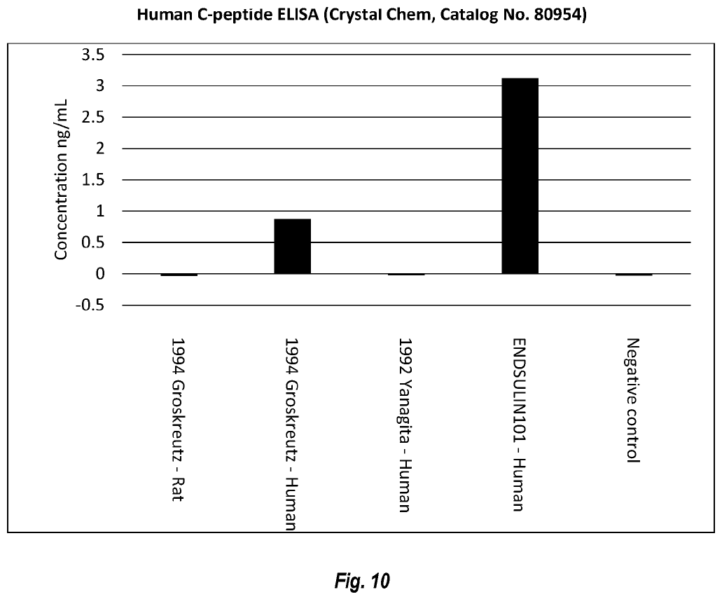

As shown by the comparison of the 1992 Yanagita-Human and 1994 Groskreutz-Human constructs to the wildtype human preproinsulin sequence in FIGS. 1-4, the mutation of amino acids within the B-chain and/or C-peptide to generate furin cleavage sites results in the production of variant forms of the final processed proteins that are mutated or truncated in vivo and in vitro. Furthermore, the ELISA analysis detailed here demonstrates that the mutations or truncations in these variant products can render them undetectable using traditional insulin and C-peptide detection ELISAs. See, e.g., FIG. 9-10, 12-14. To address these problems, alternative furin cleavable insulin constructs were designed that can be expressed and processed into both wildtype human insulin and wildtype human C-peptide allowing for detection using commercially available research and clinical assays.

### Example 2. Analysis of Furin Cleavage Sites and Processing Levels

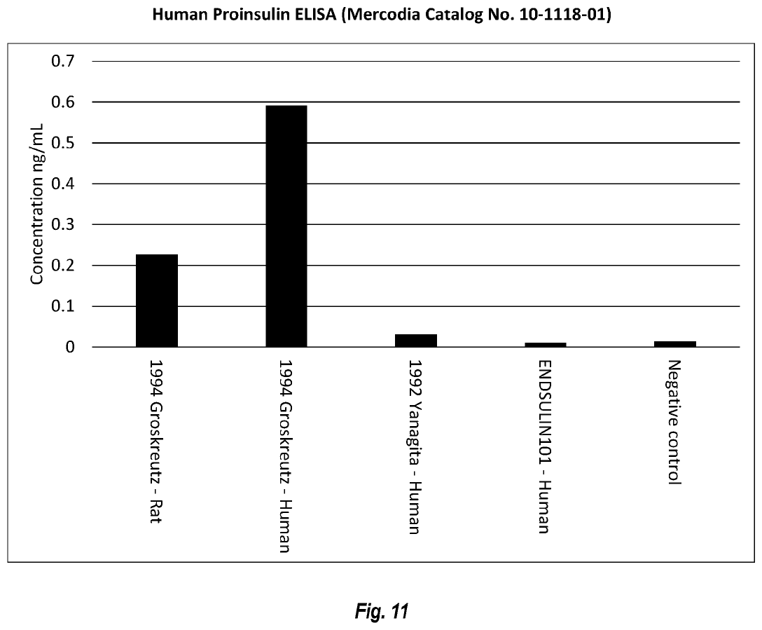

The percentage of proinsulin produced by the 1994 Groskreutz and 1992 Yanagita designs that undergoes full processing to produce mature insulin and C-peptide is less than that of wild-type proinsulin. Therefore, a percentage of the proinsulin produced by the 1994 Groskreutz and 1992 Yanagita designs accumulates as unprocessed or partially processed proinsulin (See FIG. 7B). The Mercodia Human Proinsulin ELISA (Catalog No. 10-1118-01) was used to assess the level of processing by detecting unprocessed and partially processed proinsulin produced by the 1994 Groskreutz-Rat, 1994 Groskreutz-Human and 1992 Yanagita-Human designs (FIG. 11). The processing levels reported in the literature and those obtained using the test methods of Example 4E, suggest that depending on the testing conditions, between 5-95% of the proinsulin produced using the 1994 Groskreutz and 1992 Yanagita designs does not undergo processing to form mature products.

Constructs that result in the production and accumulation of improperly processed and mutated proinsulin may be problematic when considering use as a human therapeutic. For example, cells producing these misprocessed non-wildtype proteins may become immune targets or may be subject to ER stress and cell death as seen with other misprocessed proinsulins. See FIGS. 1-4, and 6; Liu, et al. (2010); Fonseca, et al. (2011). The ELISA results of Example 4 support that not only is there a need for alternative furin cleavable insulin construct designs which can be processed into both wildtype human C-peptide and wildtype human insulin but also the need for designs where the proinsulin undergoes full and consistent processing into mature insulin and C-peptide.

The entire furin cleavage site motif is a 20-amino acid sequence represented by relative positions (e.g., P and P′) from the point of cleavage. Tian, et al. (2012). In the case of 1992 Yanagita-Human and 1994 Groskreutz-Human designs, the engineered furin cleavage sites were characterized as only a four amino acid sequence comprising the two original amino acids of the human B/C or C/A junctions, plus two flanking amino acids within either the original B-chain or C-peptide that were mutated to generate a four amino acid furin cleavage site. See Groskreutz, et al. (1994); Yanagita, et al. (1992). However, it is important to consider an engineered furin cleavage site in the larger context of the twenty amino acids surrounding the point of cleavage. The twenty amino acid proinsulin furin cleavage sites for the existing designs and exemplary inventive designs described herein, which include the original insulin B/C and C/A junction sites, are shown in Table 5. Furin is understood to cleave between P1 and P1′ of these sequences. The entirety of the 20 amino acid furin cleavage site was considered when designing the furin cleavable insulin constructs disclosed herein.

There are several “regions” to the furin cleavage site motif. The region represented by P14-11 is considered outside the binding pocket and is understood to be the solvent accessible region. The region represented by P10-P7 is also outside the furin binding pocket, and is understood to be another solvent accessible region, where small and hydrophilic residues are favored, and the polar side chains may form weak interactions with the polar surface of furin. The region represented by P6-P1 and P1′-P2′ is considered the furin binding pocket. Changes to individual amino acids in this region can have significant effects on cleavage and binding. For example, the furin binding pocket region is generally a positively charged region, wherein the presence of cysteine may reduce furin cleavage efficiency. The region represented by P3′-P6′ is considered outside the furin binding pocket and is another solvent accessible region. This region favors small and hydrophilic residues or polar side chains which may form weak interactions with the polar surface of furin.

To examine the impact of different amino acids in different positions along the sequence, the amino acids at each position, particularly in the sequence of P6-P5-P4-P3-P2-P1, were considered. The presence of specific amino acids at specific positions is thought to dictate furin binding efficiency. For example, O-linked glycosylation in the furin binding pocket (P6-P1 and P1′-P2′ region) can alter the physical properties of this region and effect furin binding strength. Significantly, the 1994 Groskreutz-Rat, 1994 Groskreutz-Human and 1992 Yanagita-Human designs are all predicted to have O-linked glycosylation modifications on the T located in either position P6 or P5 of the 20 amino acid furin cleavage site that spans their respective B-chain, B/C junction, and C-peptide sequences. Steentoft, et al. (2013). This predicted O-linked glycosylation could interfere with furin binding and cleavage and may cause decreased proinsulin processing. There are also other amino acids in this same region in the different variants which conflict with the 20 amino acid furin cleavage consensus sequence. For example, the presence of proline (P) in the P5 position of the 1994 Groskreutz-Rat and 1994 Groskreutz-Human constructs is hypothesized to be overly rigid and may disrupt the necessary structure or conformation needed for furin cleavage. While the presence of aspartic acid (D) in the P2′ position of the 1992 Yanagita-Human design is thought to dramatically increase the amount of overall negative charge in this region possibly resulting in reduced binding.

TABLE 5
Twenty-Amino Acid Furin Cleavage Sites

| Sequence | P14 | P13 | P12 | P11 | P10 | P9 | P8 | P7 | P6 | P5 | P4 | P3 | P2 | P1 | P1′ | P2′ | P3′ | P4′ | P5′ | P6′ |
|---|---|---|---|---|---|---|---|---|---|---|---|---|---|---|---|---|---|---|---|---|
| Wildtype_31_32 | C | G | E | R | G | F | F | Y | T | P | K | T | R | R | E | A | E | D | L | Q |
| SEQ ID NO: 137 | | | | | | | | | | | | | | | | | | | | |
| Wildtype_64_65 | S | L | Q | P | L | A | L | E | G | S | L | Q | K | R | G | I | V | E | Q | C |
| SEQ ID NO: 138 | | | | | | | | | | | | | | | | | | | | |
| 1994 Groskreutz- | C | G | E | R | G | F | F | Y | T | P | R | T | K | R | E | V | E | D | P | Q |
| Rat_31_32 | | | | | | | | | | | | | | | | | | | | |
| SEQ ID NO: 139 | | | | | | | | | | | | | | | | | | | | |
| 1994 Groskreutz- | D | L | Q | T | L | A | L | E | V | A | R | Q | K | R | G | I | V | D | Q | C |
| Rat_64_65 | | | | | | | | | | | | | | | | | | | | |
| SEQ ID NO: 140 | | | | | | | | | | | | | | | | | | | | |
| 1994 Groskreutz- | C | G | E | R | G | F | F | Y | T | P | R | T | K | R | E | A | E | D | L | Q |
| Human_31_32 | | | | | | | | | | | | | | | | | | | | |
| SEQ ID NO: 141 | | | | | | | | | | | | | | | | | | | | |
| 1994 Groskreutz- | S | L | Q | P | L | A | L | E | G | S | R | Q | K | R | G | I | V | E | Q | C |
| Human_64_65 | | | | | | | | | | | | | | | | | | | | |
| SEQ ID NO: 142 | | | | | | | | | | | | | | | | | | | | |
| 1992 Yanagita- | E | R | G | F | F | Y | T | P | K | T | R | R | K | R | E | D | L | Q | V | G |
| Human_31_32 | | | | | | | | | | | | | | | | | | | | |
| SEQ ID NO: 143 | | | | | | | | | | | | | | | | | | | | |
| 1992 Yanagita- | S | L | Q | P | L | A | L | E | G | S | R | R | K | R | G | I | V | E | Q | C |
| Human_64_65 | | | | | | | | | | | | | | | | | | | | |
| SEQ ID NO: 144 | | | | | | | | | | | | | | | | | | | | |
| ENDSULIN101_31_ | E | R | G | F | F | Y | T | P | K | T | R | R | K | R | E | A | E | D | L | Q |
| 32 | | | | | | | | | | | | | | | | | | | | |
| SEQ ID NO: 145 | | | | | | | | | | | | | | | | | | | | |
| ENDSULIN_64_65 | Q | P | L | A | L | E | G | S | L | Q | R | R | K | R | G | I | V | E | Q | C |
| SEQ ID NO: 146 | | | | | | | | | | | | | | | | | | | | |

### Example 3. Design of ENDSULIN101-Human

There are no furin cleavable insulin designs in the art that consistently undergo appropriate and safe proinsulin processing to generate wildtype A-chain, B-chain, and C-peptide protein products. As such, there existed a need for an engineered insulin protein which could be administered to a patient safely, was processed into wildtype insulin products, and could be monitored using existing detection methods.

To solve this problem, variant preproinsulin proteins were designed with two additional basic amino acids inserted (a) between the last amino acid of the mature B-chain and the first amino acid of the mature C-peptide (within the B/C junction); and (b) between the last amino acid of the mature C-peptide and the first amino acid of the mature A-chain (within the C/A junction), to generate functional enzymatic cleavage recognition site(s). The variant proteins were designed to be processed into wildtype insulin and wildtype C-peptide. Variant preproinsulin constructs based on this design, including SEQ ID NOs: 3-38 contain four-amino acid furin cleavage sites at both the B/C and C/A junctions. ENDSULIN 101-Human (SEQ ID NO: 31), which contains the P4-P1 Furin Cleavage Sequence 5, RRKR (SEQ ID NO: 56), as both the B/C and C/A junctions, was chosen for further development.

As part of the design process, ENDSULIN101-Human was also assessed for O-linked glycosylation and, unlike the existing designs, the only predicted O-linked glycosylation site in the B/C or C/A junctions of ENDSULIN101 is at P8 of the B/C junction which is outside the furin binding pocket (P6-P1 and P1′-P2′) and thus outside the area predicted to be negatively effected by O-linked glycosylation.

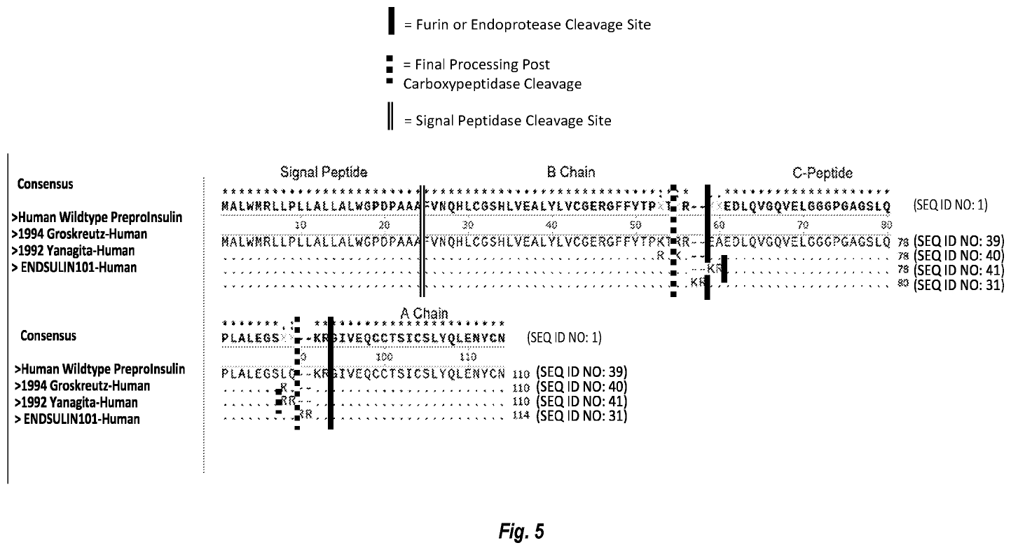

FIG. 5 shows the processing of ENDSULIN101-Human proinsulin (SEQ ID NO: 31), one of several exemplary variant proinsulin constructs described herein. As shown, the amino acid sequence of wildtype human proinsulin was modified at the processing sites such that the preproinsulin and proinsulin forms of ENDSULIN101-Human have four additional amino acids compared to wildtype human, 1994 Groskreutz-Human, and/or 1992 Yanagita-Human proinsulin constructs. However, unlike 1994 Groskreutz-Human and 1992 Yanagita-Human, the final mature insulin and mature C-peptide generated from ENDSULIN101 are identical to mature wildtype human insulin and mature wildtype human C-peptide.

Typically inserting amino acids in the middle of a protein, at one location much less two locations, results in disruption of proper protein structure, processing, and/or function. However, expression of the ENDSULIN101-Human construct, which includes the insertion of two amino acids within each junction, resulted in fully processed insulin (See Example 4E, below).

### Example 4. Expression Potency Assays

Huh-7 cells (Creative Bioarray, Catalog No. CSC-C9441L), a human liver carcinoma cell line, were cultured in a humidified incubator at 5% CO₂ and 37° C. using complete growth media (RPMI 1640 media, Gibco, Catalog No. 11875093) with the following additions: 10% heat inactivated FBS (Gibco, Catalog No. 10082147), 1× (50 U/ml) Penicillin Streptomycin (Gibco, Catalog No. 15070063) and 1× (2 mM) glutaMAX (Gibco, Catalog No. 35050061). Cells were passaged twice per week by digestion using Trypsin-EDTA (Gibco, Catalog No. 25200-056) for 5 min. at room temperature and plated at either 23,000 cells/cm² or 33,000 cells/cm² in new flasks (Corning, Catalog No. 430641U) depending on the time between passages. The day before the assay, the cells were passaged and plated into 12-well plates (GenClone, Catalog No. 25-106MP) at 86,000 cells/cm² and placed in an incubator overnight.

Using droplet digital PCR (ddPCR) titers, each vector stock was diluted to a concentration of 1e12 vg/mL in EZ-buffer (20 mM Tris-HCL pH 8, Invitrogen, Catalog No. 15568-025; 1 mM MgCl₂, Invitrogen, Catalog No. AM9530G; 200 mM NaCl, Invitrogen, Catalog No. AM9760G; and 0.005% Pluronic F-68 Polyol, MP Biomedicals, Catalog No. 2750049) before being diluted to a final dilution of 4.8e10 vg/mL. The final dilution represents a multiplicity of infection (MOI) of 1.6e5 vg/cell in transduction media (RPMI-1640 supplemented with 1× (2 mM) Glutamax and 0.001% Pluronic F-68 Polyol).

Vectors used for transduction were 1994 Groskreutz-Rat, 1994 Groskreutz-Human, 1992 Yanagita-Human, ENDSULIN101-Human, and a negative control vector. The plasmids used to generate the vector genomes of these viruses include the ITR-ITR nucleotide sequences of SEQ ID NO: 183 (1994 Groskreutz-Rat), SEQ ID NO: 184 (1994 Groskreutz-Human), SEQ ID NO: 185 (1992 Yanagita-Human), SEQ ID NO: 76 (ENDSULIN101-Human), and SEQ ID NO: 186 (negative control) and were all in a truncated pUC57 backbone except for 1994 Groskreutz-Rat whose entire vector sequences is from P210118US02 (SEQ ID NO:1). For transduction, the culture media was replaced with transduction media containing diluted vector and the cells were placed in the incubator for at least 20 h. The next day the transduction media was replaced with expression media (RPMI-1640 supplemented with 1× (2 mM) Glutamax) and placed in the incubator for 24 h. The following day the expression media was harvested and placed in 1.5 mL tubes.

Harvested samples were analyzed using ELISAs for the production of proinsulin and its various partially and fully processed forms. All experiments were performed more than once and the results presented here each represent the average of two replicates from a single experiment, The ELISAs were run according to the manufacturers' instructions with the following exceptions: the number of washes were increased to 12 for the Mercodia assays and to 8 for the Crystal Chem assay. Absorbances for all the ELISA assays were read according to the manufacturers' instructions using a Promega GloMax Discover Plate Reader. Sample concentrations were calculated as recommended in the manufacturers using the calibrators and cubic spline regressions.

### Example 5. Testing of Preproinsulin Constructs

### A. Assay Selection

The protein expression products generated using viruses containing the preproinsulin constructs were tested using a variety of ELISA kits to determine which unprocessed, partially processed, and fully processed forms of proinsulin were produced. In particular, the products from the 1994 Groskreutz-Rat, 1994 Groskreutz-Human, 1992 Yanagita-Human, and ENDSULIN101-Human (SEQ ID NOs: 42, 40, 41, and 31, respectively) viruses were analyzed for the quantity of mature human insulin (Insulin ELISA, Mercodia, Catalog No. 10-1113-01), human C-peptide (C-peptide ELISA, Mercodia, Catalog No. 10-1136-01 and C-peptide ELISA Kit, Crystal Chem, Catalog No. 80954), proinsulin (Proinsulin ELISA, Mercodia, Catalog No. 10-1118-01), all insulin species (Iso-Insulin ELISA, Mercodia, Catalog No. 10-1128-01), rat insulin (Rat Insulin ELISA, Mercodia, Catalog No. 10-1250-01), and rat C-peptide (Rat C-peptide ELISA, Mercodia, Catalog No. 10-1172-01) using ELISA assays. The specificities of the ELISA assays, where available, are provided in Table 6 below.

TABLE 6

| | Mature | | | | | | |
|---|---|---|---|---|---|---|---|
| | Human | Human C- | Human C- | | Iso- | Rat | Rat C- |
| | Insulin | peptide | peptide | Proinsulin | Insulin | Insulin | peptide |
| | ELISA | ELISA | ELISA | ELISA | ELISA | ELISA | ELISA |
| | (Mercodia | (Mercodia | (Crystal | (Mercodia | (Mercodia | (Mercodia | (Mercodia |
| Specificity | 10-1113-01) | 10-1136-01) | Chem 80954) | 10-1118-01) | 10-1128-01) | 10-1250-01) | 10-1172-01) |
| Human Insulin | 100% | <0.0006% | | <0.03% | 100% | 167% | |
| Human C-peptide | <0.01% | 100% | 100% | <0.006% | <0.1% | <0.05% | n.d. |
| Rat C-Peptide | | | | | | <0.001% | 100% |
| Mouse C-Peptide | | | | | | | 6% |
| Human Proinsulin | <0.01% | 2% | | 100% | 54% | 75% | |
| Rat Proinsulin (unspecified) | | | | | | 7% | 4.6% |
| Rat Proinsulin I | | | | 13% | | | |
| Rat Proinsulin II | | | | 10% | | | |
| Human Proinsulin des (31-32) | <0.5% | 3% | | 95% | 58% | | |
| Human Proinsulin split (32-33) | <0.5% | 2% | | 95% | 56% | | |
| Human Proinsulin des (64-65) | 98% | 74% | | 84% | 66% | | |
| Human Proinsulin split (65-66) | 56% | 10% | | 90% | 78% | | |
| Human Insulin lispro | n.d. | | | | 112% | 167% | |
| Human Insulin aspart | 4% | | | | 100% | | |
| Human Insulin determir | n.d. | | | | 28% | | |
| Human Insulin glargine | 24% | | | | 58% | | |
| Human Insulin glulisine | n.d. | | | | 123% | | |
| Human IGF-I | <0.02% | | | | <0.02% | | |
| Human IGF-II | <0.02% | | | | <0.02% | | |
| Porcine Proinsulin | | | | 4% | | | |
| Mouse Proinsulin I | | | | 3% | | | |
| Mouse Proinsulin II | | | | 16% | | | |
| Rat insulin | 0.7% | | | | 71% | 100% | n.d. |
| Mouse insulin | 0.3% | | | | 49% | 75% | |
| Porcine insulin | 93% | | | | 76% | | |
| Ovine insulin | 48% | | | | 131% | | |
| Bovine insulin | 31% | | | | 58% | | |
| Porcine C-peptide | | | | | | | n.d. |

n.d. = not detected;
blank cell = data not available

### B. Mature Human Insulin ELISA (Mercodia, Catalog No. 10-1113-01)

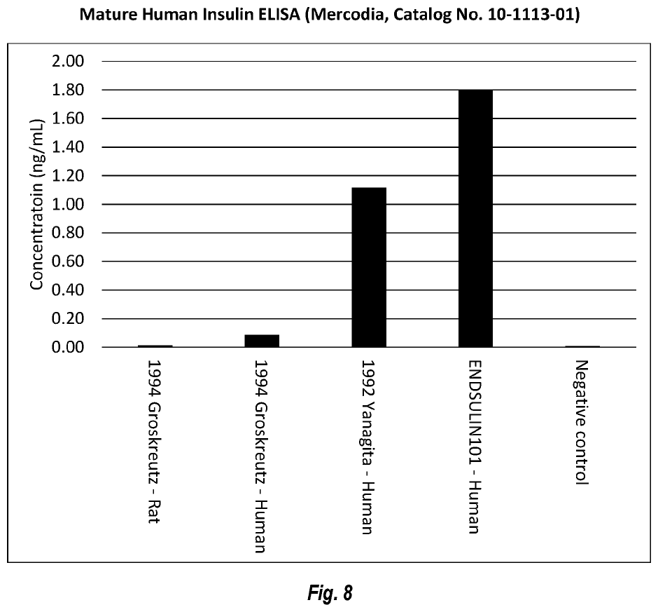

The protein expression products generated using viruses containing the preproinsulin constructs 1994 Groskreutz-Rat, 1994 Groskreutz-Human, 1992 Yanagita-Human, ENDSULIN101-Human, and a negative control were tested using the Mercodia Insulin ELISA (Catalog No. 10-1113-01). FIG. 8 shows the results of the assay. Media from cells transduced with the 5 different vectors (x-axis) was quantified for the presence of mature human insulin in ng/mL (y-axis). As shown in FIG. 8, ENDSULIN101-Human and 1992 Yanagita-Human produced mature wildtype human insulin. The ELISA was unable to recognize the mature insulin produced by the 1994 Groskreutz-Human design due to the amino acid mutations in the B-chain (e.g., see FIG. 3).

### C. Human C-peptide ELISA (Mercodia, Catalog No. 10-1136-01)

The protein expression products generated using viruses containing the preproinsulin constructs 1994 Groskreutz-Rat, 1994 Groskreutz-Human, 1992 Yanagita-Human, ENDSULIN101-Human, and a negative control were tested using the Mercodia C-peptide ELISA (Catalog No. 10-1136-01), which is an immunoassay for specific quantitation of human C-peptide that can be used with serum, plasma, and urine samples. FIG. 9 shows the results of the assay. Media from cells transduced with 5 different vectors (x-axis) was quantified for the presence of human C-peptide in ng/mL (y-axis). As shown in FIG. 9, ENDSULIN101-Human produced mature human C-peptide. This ELISA was unable to recognize the C-peptide produced from the 1994 Groskreutz-Human design due to mutations in the amino acid sequence of the C-peptide (e.g., see FIG. 4). Additionally, the ELISA was unable to recognize the C-peptide produced from the 1992 Yanagita-Human design due to truncations in the C-peptide (e.g., see FIG. 4).

### D. Human C-peptide ELISA (Crystal Chem, Catalog No. 80954)

The protein expression products generated using viruses containing the preproinsulin constructs 1994 Groskreutz-Rat, 1994 Groskreutz-Human, 1992 Yanagita-Human, ENDSULIN101-Human, and a negative control were tested using the Crystal Chem C-peptide ELISA (Catalog No. 80954), a highly sensitive assay used to quantify levels of human C-peptide in serum and plasma. FIG. 10 shows the results of the assay. Media from cells transduced with 5 different vectors (x-axis) was quantified for the presence of human C-peptide in ng/mL (y-axis). As shown in FIG. 10, ENDSULIN101-Human produced mature human C-peptide. This ELISA was not able to recognize the truncated C-peptide of the 1992 Yanagita-Human design, but did recognize the mutated C-peptide of the 1994 Groskreutz-Human design suggesting that the epitopes recognized by this ELISA do not include the amino acids mutated in the 1994 Groskreutz-Human design.

### E. Human Proinsulin ELISA (Mercodia Catalog No. 10-1118-01)

The protein expression products generated using viruses containing the preproinsulin constructs 1994 Groskreutz-Rat, 1994 Groskreutz-Human, 1992 Yanagita-Human, ENDSULIN101-Human, and a negative control were tested in a Mercodia Proinsulin ELISA (Catalog No. 10-1118-01), which is an assay used for quantifying the levels of human proinsulin in serum or plasma. FIG. 11 shows the results of the assay. Media from cells transduced with 5 different vectors (x-axis) was quantified for the presence of proinsulin in ng/mL (y-axis). As shown in FIG. 11, the low level of proinsulin detected from the ENDSULIN101-Human design using this proinsulin ELISA was less than the background level detected for the negative control construct suggesting complete or near complete processing, while 1994 Groskreutz-Human, 1994 Groskreutz-Rat, and 1992 Yanagita-Human designs all demonstrated notable amounts of unprocessed or partially processed proinsulin which is consistent with prior published data from multiple groups.

### F. Iso-Insulin ELISA (Mercodia, Catalog No. 10-1128-01)

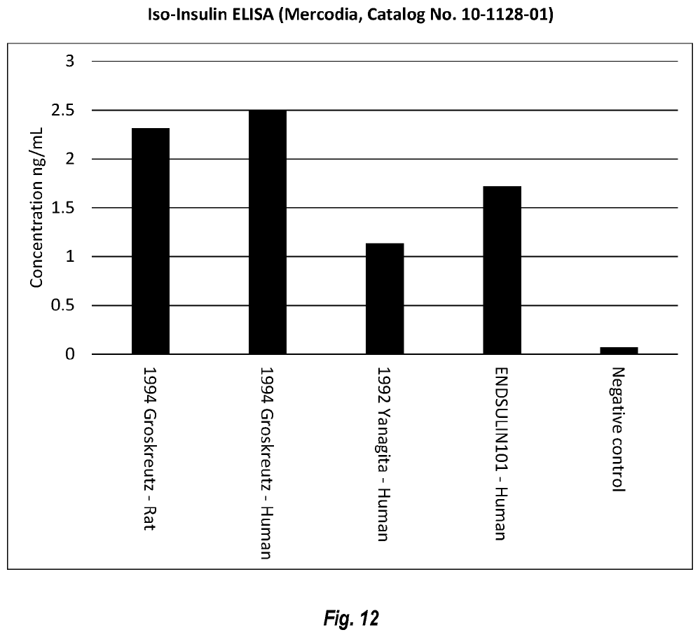

The protein expression products generated using viruses containing the preproinsulin constructs 1994 Groskreutz-Rat, 1994 Groskreutz-Human, 1992 Yanagita-Human, ENDSULIN101-Human, and a negative control were tested in a Mercodia Iso-Insulin ELISA (Catalog No. 10-1128-01). FIG. 12 shows the results of the assay. Media from cells transduced with 5 different vectors (x-axis) was quantified for the presence of any insulin species regardless of processing or species. The y-axis represents the concentration in ng/mL. As shown in FIG. 12, the Iso-Insulin ELISA showed that cells transduced with all of the designs strongly produced various forms of insulin compared to the negative control design (1994 Groskreutz-Human ATG minus).

It is important to note that the Iso-Insulin ELISA not only recognized mature insulin, but also recognized proinsulin, and all of the processing intermediates. Furthermore, each of these forms is recognized by the ELISA with a different binding affinity (Table 7). Additionally, this ELISA recognizes the products of the various constructs with different binding affinities due to species' differences (rat vs. human) and possibly also due to differences in how the antibodies bind the mutated or unmutated forms present. For example, the Iso-Insulin ELISA kit detects 1 ng/mL of human insulin as 1 ng/mL, but 1 ng/mL of rat insulin is only detected as approximately 0.71 ng/mL (see Table 6 listing specificity for human insulin as 100% versus 71% specificity for rat insulin). Thus, while the production of various forms of insulin is detected for all four constructs, direct comparisons of the relative protein production levels from the different designs is not possible.

### G. Rat Insulin ELISA (Mercodia, Catalog No. 10-1250-01)

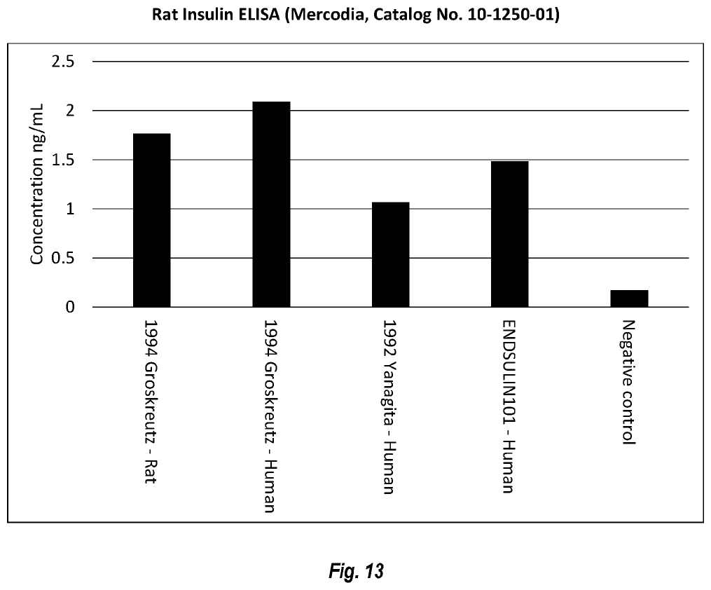

The protein expression products generated using viruses containing the preproinsulin constructs 1994 Groskreutz-Rat, 1994 Groskreutz-Human, 1992 Yanagita-Human, ENDSULIN101-Human, and a negative control were also tested using the Mercodia Rat insulin Insulin ELISA (Catalog No. 10-1250-01) which is a pan-insulin ELISA. FIG. 13 shows the results of the assay. Media from cells transduced with 5 different vectors (x-axis) was quantified for the presence of rat or human insulin in ng/mL using the Mercodia Rat insulin ELISA assay. The y-axis represents the concentration in ng/mL.

As shown in FIG. 13, the Rat Insulin ELISA showed that cells transduced with each of the designs besides the negative control design (1994 Groskreutz-Human-ATG minus) strongly produced various forms of insulin compared to the negative control design (1994 Groskreutz-Human ATG minus). The Rat Insulin ELISA is understood to be closely related to the Iso-Insulin ELISA (Mercodia, Catalog No. 10-1128-01) and thus likely also recognizes the partially processed forms of proinsulin (information courtesy of Mercodia). Direct comparisons of the relative protein production levels from the different designs are also not possible using this ELISA.

### H. Rat C-peptide ELISA (Mercodia, Catalog No. 10-1172-01)

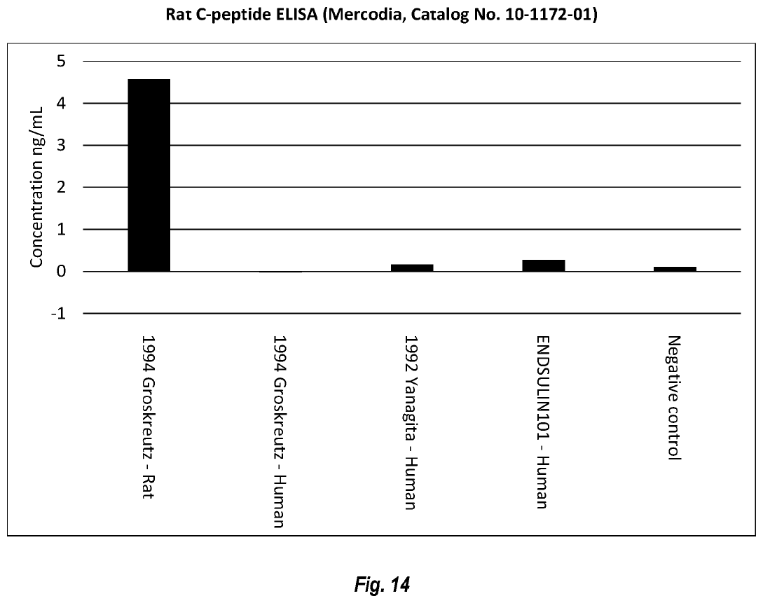

The protein expression products generated using viruses containing the preproinsulin constructs 1994 Groskreutz-Rat, 1994 Groskreutz-Human, 1992 Yanagita-Human, ENDSULIN101-Human, and a negative control were further tested in a Mercodia Rat C-peptide ELISA (Catalog No. 10-1172-01). This ELISA is specific to rat C-peptide and its partially processed forms and is not expected to cross-react with any of the unprocessed, partially processed or fully processed human C-peptide. FIG. 14 shows the results of the assay. Media from cells transduced with 5 different vectors (x-axis) was quantified for the presence of rat C-peptide in ng/mL (y-axis). As shown in FIG. 14, the result confirmed that the 1994 Groskreutz-Rat design produced at least partially processed rat C-peptide.

### I. Examination of Additional ENDSULIN101-Like Constructs

It is understood that the additional proinsulin constructs with SEQ ID NOs: 3-30 and 32-38 could be tested in the assays described above.

Because the proinsulin constructs given by SEQ ID NOs: 3-30 and 32-38 comprise a similar design to ENDSULIN101-Human (SEQ ID NO: 31), that is, comprise basic amino acids that are inserted into each of the B/C and C/A junctions of a proinsulin construct to generate new proteins which could be processed in vivo and in vitro into wildtype insulin and C-peptide, it is understood that the constructs are expected to have similar results to ENDSULIN101-Human. For each of SEQ ID NOs: 3-30 and 32-38, the final mature insulin and mature C-peptide generated should be identical to mature wildtype human insulin and mature wildtype human C-peptide.

### Example 6. ENDSULIN101 Analog Constructs

The results reported in Example 4 suggest that ENDSULIN101-Human generates mature wildtype insulin and mature C-peptide while simultaneously accumulating less than 1% unprocessed proinsulin. Additional human preproinsulin constructs were designed based on the ENDSULIN101-Human format. For these designs, the variant B/C and the variant C/A junctions, which replace the 2 amino acid wildtype B/C and C/A junctions, each comprise from 4 to 10 amino acids selected from histidine, lysine, and arginine, wherein the variant B/C and the variant C/A junctions each comprise a functional enzymatic cleavage site capable of being cleaved after the C-terminal amino acid of the junction. The following formula may be used for designing variant preproinsulin and proinsulin constructs having variant B/C and C/A junctions with four-amino acid furin cleavage sites: XₙRX₁X₂R, wherein Xₙ is 0, 1, 2, 3, 4, 5, or 6 amino acids each selected from H, K, and R; wherein X₁ is H, K, or R; and wherein X₂ is R or K. Exemplary constructs are included in Table 1 (e.g., SEQ ID Nos: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 47,50, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 74, or 75).

Examples of functional variant B/C and variant C/A junctions, including possible 4-6 amino acid junctions, are recited in Table 7. In the B/C and C/A examples shown below, the bolded residues indicate the four amino acids at the C-terminal end of the junctions that correspond to the P4-P1 residues of the 20 amino acid furin cleavage site. Besides the six different four-amino acid exemplary variant junctions (SEQ ID 52-57), Table 7 also includes exemplary additions of 1 or 2 basic amino acids to the amino end of the six possible combinations of four P4-P1 residues (SEQ ID 52-57). The addition of 3, 4, 5, or 6 amino acids at the amino end of the six four-amino acid exemplary variant junctions containing the P4-P1 residues would follow the same approach. For example, 0, 1, 2, 3, 4, 5, or 6 amino acids may be added at the N-terminus of each of the four amino acid furin cleavable variant exemplary junctions provided the additional residues are selected from histidine, lysine, and arginine, which are the targets of carboxypeptidase. Additionally, the specificity of the cleavage may be more accurate when the 4 C-terminal amino acids of the variant B/C junction and the 4 C-terminal amino acids of the variant C/A junction each are the only amino acids in the junction that generate a predicted enzymatic cleavage site and there are no other predicted enzymatic cleavage sites within each variant junction. Since the junctions were shown with the ENDSULIN101 design to accommodate additional amino acids without compromising protein processing, variant junctions comprising a track of 4-10 basic amino acids may form a flexible, disordered region (i.e., not an alpha helix or beta sheet) at the junctions, much like a HIS-tag, and have minimal effect on protein folding, processing, and/or activity of the variant preproinsulin and proinsulin constructs.

TABLE 7
Modified Furin Cleaveage Sites for ENSULIN101-Human

| Description | Sequence | SEQ ID NO |
|---|---|---|
| Exemplary Variant Junction Sequence 1 | RHKR | 52 |
| Exemplary Variant Junction Sequence 2 | RHRR | 53 |
| Exemplary Variant Junction Sequence 3 | RKKR | 54 |
| Exemplary Variant Junction Sequence 4 | RKRR | 55 |
| Exemplary Variant Junction Sequence 5 | RRKR | 56 |
| Exemplary Variant Junction Sequence 6 | RRRR | 57 |
| Exemplary Variant Junction Sequence 7 | HRHKR | 78 |
| Exemplary Variant Junction Sequence 8 | HRHRR | 79 |
| Exemplary Variant Junction Sequence 9 | HRKKR | 80 |
| Exemplary Variant Junction Sequence 10 | HRKRR | 81 |
| Exemplary Variant Junction Sequence 11 | HRRKR | 82 |
| Exemplary Variant Junction Sequence 12 | HRRRR | 83 |
| Exemplary Variant Junction Sequence 13 | KRHKR | 84 |
| Exemplary Variant Junction Sequence 14 | KRHRR | 85 |
| Exemplary Variant Junction Sequence 15 | KRKKR | 86 |
| Exemplary Variant Junction Sequence 16 | KRKRR | 87 |
| Exemplary Variant Junction Sequence 17 | KRRKR | 88 |
| Exemplary Variant Junction Sequence 18 | KRRRR | 89 |
| Exemplary Variant Junction Sequence 19 | RRHKR | 90 |
| Exemplary Variant Junction Sequence 20 | RRHRR | 91 |
| Exemplary Variant Junction Sequence 21 | RRKKR | 92 |
| Exemplary Variant Junction Sequence 22 | RRRKR | 93 |
| Exemplary Variant Junction Sequence 23 | RRKRR | 187 |
| Exemplary Variant Junction Sequence 24 | RRRRR | 188 |
| Exemplary Variant Junction Sequence 25 | HHRHKR | 94 |
| Exemplary Variant Junction Sequence 26 | HHRHRR | 95 |
| Exemplary Variant Junction Sequence 27 | HHRKKR | 96 |
| Exemplary Variant Junction Sequence 28 | HHRKRR | 97 |
| Exemplary Variant Junction Sequence 29 | HHRRKR | 98 |
| Exemplary Variant Junction Sequence 30 | HHRRRR | 99 |
| Exemplary Variant Junction Sequence 31 | HKRHKR | 100 |
| Exemplary Variant Junction Sequence 32 | HKRHRR | 101 |
| Exemplary Variant Junction Sequence 33 | HKRKKR | 102 |
| Exemplary Variant Junction Sequence 34 | HKRKRR | 103 |
| Exemplary Variant Junction Sequence 35 | HKRRKR | 104 |
| Exemplary Variant Junction Sequence 36 | HKRRRR | 105 |
| Exemplary Variant Junction Sequence 37 | HRRHKR | 106 |
| Exemplary Variant Junction Sequence 38 | HRRHRR | 107 |
| Exemplary Variant Junction Sequence 39 | HRRKKR | 108 |
| Exemplary Variant Junction Sequence 40 | HRRRKR | 109 |
| Exemplary Variant Junction Sequence 41 | KHRHKR | 110 |
| Exemplary Variant Junction Sequence 42 | KHRHRR | 111 |
| Exemplary Variant Junction Sequence 43 | KHRKKR | 112 |
| Exemplary Variant Junction Sequence 44 | KHRKRR | 113 |
| Exemplary Variant Junction Sequence 45 | KHRRKR | 114 |
| Exemplary Variant Junction Sequence 46 | KHRRRR | 115 |
| Exemplary Variant Junction Sequence 47 | KKRHKR | 116 |
| Exemplary Variant Junction Sequence 48 | KKRHRR | 117 |
| Exemplary Variant Junction Sequence 49 | KKRKKR | 118 |
| Exemplary Variant Junction Sequence 50 | KKRKRR | 119 |
| Exemplary Variant Junction Sequence 51 | KKRRKR | 120 |
| Exemplary Variant Junction Sequence 52 | KKRRRR | 121 |
| Exemplary Variant Junction Sequence 53 | KRRHKR | 122 |
| Exemplary Variant Junction Sequence 54 | KRRHRR | 123 |
| Exemplary Variant Junction Sequence 55 | KRRKKR | 124 |
| Exemplary Variant Junction Sequence 56 | KRRRKR | 125 |
| Exemplary Variant Junction Sequence 57 | RHRHKR | 126 |
| Exemplary Variant Junction Sequence 58 | RHRHRR | 127 |
| Exemplary Variant Junction Sequence 59 | RHRKKR | 128 |
| Exemplary Variant Junction Sequence 60 | RHRKRR | 129 |
| Exemplary Variant Junction Sequence 61 | RKRHKR | 130 |
| Exemplary Variant Junction Sequence 62 | RKRHRR | 131 |
| Exemplary Variant Junction Sequence 63 | RKRKKR | 132 |
| Exemplary Variant Junction Sequence 64 | RKRKRR | 133 |
| Exemplary Variant Junction Sequence 65 | RRRHKR | 134 |
| Exemplary Variant Junction Sequence 66 | RRRHRR | 135 |
| Exemplary Variant Junction Sequence 67 | RRRKKR | 136 |
| Exemplary Variant Junction Sequence 68 | HRRKRR | 189 |
| Exemplary Variant Junction Sequence 69 | HRRRRR | 190 |
| Exemplary Variant Junction Sequence 70 | KRRKRR | 191 |
| Exemplary Variant Junction Sequence 71 | KRRRRR | 192 |
| Exemplary Variant Junction Sequence 72 | RHRRKR | 193 |
| Exemplary Variant Junction Sequence 73 | RHRRRR | 194 |
| Exemplary Variant Junction Sequence 74 | RKRRKR | 195 |
| Exemplary Variant Junction Sequence 75 | RKRRRR | 196 |
| Exemplary Variant Junction Sequence 76 | RRRKRR | 197 |
| Exemplary Variant Junction Sequence 77 | RRRRKR | 198 |
| Exemplary Variant Junction Sequence 78 | RRRRRR | 199 |

Additionally, under some circumstances, modifications to the human wildtype insulin sequence may also be desired. One such modification is the His-to-Asp mutation at position 10 in the B-chain which is believed to increase the stability of mature insulin and increase insulin's affinity for its receptor. Groskreutz et al. (1994).

Cat and dog variant preproinsulin and proinsulin constructs were also designed with variant B/C and C/A junctions using the following formula: XₙRX₁X₂R, wherein Xₙ is 0, 1, 2, 3, 4, 5, or 6 amino acids each selected from H, K, and R; wherein X₁ is H, K, or R; and wherein X₂ is R or K. For example, see Table 1, SEQ ID NO: 47, 50, 74, or 75.

### Example 7. Processing Ratios of Various Constructs

Liquid chromatography with tandem mass spectrometry (LC/MS-MS) is a powerful analytical chemistry technique that combines the physical separation capabilities of liquid chromatography with the mass analysis capabilities of mass spectrometry. Concentrated protein expression products generated using viral vectors containing the 1994 Groskreutz-Rat, 1994 Groskreutz-Human, 1992 Yanagita-Human, ENDSULIN101-Human, and negative control constructs are analyzed using an LC/MS-MS protocol to identify the various protein products produced, as well as their relative ratios, and to assist in determining the presence of any posttranslational modifications.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

### REFERENCES

The complete disclosures of all publications cited herein are incorporated herein by reference in their entireties as if each were individually set forth in full herein and incorporated.
Alam, Tausif, and Hans W. Sollinger. “Glucose-regulated insulin production in hepatocytes1.” Transplantation 74.12 (2002): 1781-1787.
Bergeron, F., R. Leduc, and R. Day. “Subtilase-like pro-protein convertases: from molecular specificity to therapeutic applications.” Journal of Molecular Endocrinology 24.1 (2000): 1-22.
Breuer C B, et al, “In vivo engineering of lymphocytes after systemic exosome-associated AAV delivery.” Sci Rep. 2020, 10(1):4544, doi: 10.1038/s41598-020 61518-w.
Cecchini S, et al., “Reproducible high yields of recombinant adeno-associated virus produced using invertebrate cells in 0.02- to 200-liter cultures.” Hum Gene Ther. 2011, 22(8):1021-30.
Carson, Mike, et al. “His-tag impact on structure.” Acta Crystallographica Section D: Biological Crystallography 63.3 (2007): 295-301.
Duckert, Peter, Soren Brunak, and Nikolaj Blom. “Prediction of proprotein convertase cleavage sites.” Protein Engineering Design and Selection 17.1 (2004): 107-112.
Fonseca, Sonya G., Jesper Gromada, and Fumihiko Urano. “Endoplasmic reticulum stress and pancreatic β-cell death.” Trends in Endocrinology & Metabolism 22.7 (2011): 266-274.
Grimm D, et al., “Novel tools for production and purification of recombinant adenoassociated virus vectors.” Hum Gene Ther. 1998, 9(18):2745-60.
Groskreutz, Debyra J., Mark X. Sliwkowski, and Cornelia M. Gorman. “Genetically engineered proinsulin constitutively processed and secreted as mature, active insulin.” Journal of Biological Chemistry 269.8 (1994): 6241-6245.
Hafenrichter, D. G., Wu, X., Rettinger, S. D., Kennedy, S. C., Flye, M. W., & Ponder, K. P. (1994). Quantitative evaluation of liver-specific promoters from retroviral vectors after in vivo transduction of hepatocytes. Blood, 84(10), 3394-3404.
Hay, Colin William, and Kevin Docherty. “Enhanced expression of a furin-cleavable proinsulin.” Journal of molecular endocrinology 31.3 (2003): 597-607.
Heard, Jean-Michel, et al. “Determinants of rat albumin promoter tissue specificity analyzed by an improved transient expression system.” Molecular and Cellular Biology 7.7 (1987): 2425-2434.
Hermonat P L, et al., “The packaging capacity of adeno-associated virus (AAV) and the potential for wild-type-plus AAV gene therapy vectors.” FEBS Lett. 1997c, 407(1):78-84.
Iwaki, Hirohisa, et al. “Fluorescence Probes for Imaging Basic Carboxypeptidase Activity in Living Cells with High Intracellular Retention.” Analytical Chemistry 93.7 (2021): 3470-3476.
Lacy, Paul E., and David W. Scharp. “Islet transplantation in treating diabetes.” Annual review of medicine 37.1 (1986): 33-40.
Liu, Ming, et al. “Mutant INS-gene induced diabetes of youth: proinsulin cysteine residues impose dominant-negative inhibition on wild-type proinsulin transport.” PloS one 5.10 (2010): e13333.
Nakayama, K. Furin: a mammalian subtilisin/Kex2p-like endoprotease involved in processing of a wide variety of precursor proteins. Biochem. J. 327 (Pt3), 625-635 (1997).
Riu, Efren, et al. “Counteraction of type 1 diabetic alterations by engineering skeletal muscle to produce insulin: insights from transgenic mice.” Diabetes 51.3 (2002): 704-711.
Shirley, Jamie L., et al. “Immune responses to viral gene therapy vectors.” Molecular Therapy 28.3 (2020): 709-722.
Short, Daniel K., et al. “Adenovirus-mediated transfer of a modified human proinsulin gene reverses hyperglycemia in diabetic mice.” American Journal of Physiology-Endocrinology andMetabolism 275.5 (1998): E748-E756.
Simonson, Gregg D., et al. “Synthesis and processing of genetically modified human proinsulin by rat myoblast primary cultures.” Human Gene Therapy 7.1 (1996): 71-78.
Skidgel, Randal A. “Basic carboxypeptidases: regulators of peptide hormone activity.” Trends in pharmacological sciences 9.8 (1988): 299-304.
Steentoft, Catharina, et al. “Precision mapping of the human O-GalNAc glycoproteome through SimpleCell technology.” The EMBO journal 32.10 (2013): 1478-1488.
Støy, Julie, et al. “Insulin gene mutations as a cause of permanent neonatal diabetes.” Proceedings of the National Academy of Sciences 104.38 (2007): 15040-15044.
Tian, Sun, Wang Huajun, and Jianhua Wu. “Computational prediction of furin cleavage sites by a hybrid method and understanding mechanism underlying diseases.” Scientific reports 2.1 (2012): 1-7.
Yamasaki, Koichiro, et al. “Differentiation-induced insulin secretion from nonendocrine cells with engineered human proinsulin cDNA.” Biochemical and biophysical research communications 265.2 (1999): 361-365.
Yanagita, Masahiko, Kazuhisa Nakayama, and Toshiyuki Takeuchi. “Processing of mutated proinsulin with tetrabasic cleavage sites to bioactive insulin in the non-endocrine cell line, COS-7.” FEBS letters 311.1 (1992): 55-59.
Yang, Yanwu, et al. “Solution structure of proinsulin: connecting domain flexibility and prohormone processing.” Journal of Biological Chemistry 285.11 (2010): 7847-7851.

What is claimed is:

1. A nucleic acid molecule comprising a nucleic acid sequence encoding a variant preproinsulin polypeptide comprising:
a) an N-terminal signal sequence,
b) a wildtype B-chain or full-length variant thereof comprising at least one amino acid substitution,
c) a variant B/C junction comprising at least 4 amino acids selected from histidine, lysine, and arginine,
d) a wildtype C-peptide or full-length variant thereof comprising at least one amino acid substitution,
e) a variant C/A junction comprising at least 4 amino acids selected from histidine, lysine, and arginine, and
f) a wildtype A-chain or full-length variant thereof comprising at least one amino acid substitution,
wherein the variant B/C junction and the variant C/A junction each comprise an enzymatic cleavage site for a target proteolytic enzyme to cleave immediately after the C-terminal amino acid of the junction.

2. The nucleic acid molecule of claim 1, wherein the enzymatic cleavage site is a subtilisin-like proprotein convertase cleavage site.

3. The nucleic acid molecule of claim 1 or claim 2, wherein the enzymatic cleavage site is a furin cleavage site.

4. The nucleic acid molecule of any one of the preceding claims, wherein the preproinsulin polypeptide is capable of being processed into a mature wildtype insulin protein or full-length variant thereof comprising at least one amino acid substitution, and a mature wildtype C-peptide or full-length variant thereof comprising at least one amino acid substitution.

5. The nucleic acid molecule of any one of the preceding claims, wherein the preproinsulin polypeptide is capable of being processed by furin and a carboxypeptidase into a mature wildtype insulin protein or full-length variant thereof comprising at least one amino acid substitution, and a mature wildtype C-peptide or full-length variant thereof comprising at least one amino acid substitution.

6. The nucleic acid molecule of claim 4 or claim 5, wherein the mature wildtype insulin protein is a mature wildtype human insulin protein, mature wildtype canine insulin protein, or mature wildtype feline insulin protein; and wherein the mature wildtype C-peptide is a mature wildtype human C-peptide, mature wildtype canine C-peptide, or mature wildtype feline C-peptide.

7. The nucleic acid molecule of any one of claims 4 to 6, wherein the mature wildtype insulin protein is a mature wildtype human insulin protein; and wherein the mature wildtype C-peptide is a mature wildtype human C-peptide.

8. The nucleic acid molecule of any one of the preceding claims, wherein the enzymatic cleavage site comprises an amino acid sequence of RX₁X₂R (SEQ ID NO: 45), wherein X₁ is histidine, lysine, or arginine and X₂ is lysine or arginine.

9. The nucleic acid molecule of any one of the preceding claims, wherein each enzymatic cleavage site comprises an amino acid sequence selected from RHKR (SEQ ID NO: 52), RHRR (SEQ ID NO: 53), RKKR (SEQ ID NO: 54), RKRR (SEQ ID NO: 55), RRKR (SEQ ID NO: 56), and RRRR (SEQ ID NO: 57).

10. The nucleic acid molecule of any one of the preceding claims, wherein each enzymatic cleavage site comprises an amino acid sequence of RRKR (SEQ ID NO: 56).

11. The nucleic acid molecule of any one of the preceding claims, wherein the variant B/C junction and the variant C/A junction each comprise 4 to 10 amino acids selected from histidine, lysine, and arginine.

12. The nucleic acid molecule of any one of the preceding claims, wherein the variant B/C junction and the variant C/A junction each comprise 4 to 6 amino acids selected from histidine, lysine, and arginine.

13. The nucleic acid molecule of any one of the preceding claims, wherein the variant B/C junction and the variant C/A junction each comprise 4 amino acids selected from histidine, lysine, and arginine.

14. The nucleic acid molecule of any one of the preceding claims, wherein the variant B/C junction comprises an amino acid sequence of XₙRX₁X₂R (SEQ ID NO: 64), wherein Xₙ is 0, 1, 2, 3, 4, 5, or 6 amino acids each chosen from histidine, lysine, or arginine, X₁ is histidine, lysine, or arginine, and X₂ is lysine or arginine.

15. The nucleic acid molecule of any one of the preceding claims, wherein the variant B/C junction comprises an amino acid sequence of SEQ ID NO: 52, SEQ ID NO: 53, SEQ ID NO: 54, SEQ ID NO: 55, SEQ ID NO: 56, SEQ ID NO: 57, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, or SEQ ID NO: 199.

16. The nucleic acid molecule of any one of the preceding claims, wherein the variant B/C junction comprises an amino acid sequence of SEQ ID NO: 52, SEQ ID NO: 53, SEQ ID NO: 54, SEQ ID NO: 55, SEQ ID NO: 56, SEQ ID NO: 57, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, or SEQ ID NO: 136.

17. The nucleic acid molecule of any one of the preceding claims, wherein the variant B/C junction comprises an amino acid sequence of RRKR (SEQ ID NO: 56).

18. The nucleic acid molecule of any one of the preceding claims, wherein the variant C/A junction comprises an amino acid sequence of XₙRX₁X₂R (SEQ ID NO: 64), wherein Xₙ is 0, 1, 2, 3, 4, 5, or 6 amino acids each chosen from histidine, lysine, or arginine, X₁ is histidine, lysine, or arginine, and X₂ is lysine or arginine.

19. The nucleic acid molecule of any one of the preceding claims, wherein the variant C/A junction comprises an amino acid sequence of SEQ ID NO: 52, SEQ ID NO: 53, SEQ ID NO: 54, SEQ ID NO: 55, SEQ ID NO: 56, SEQ ID NO: 57, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 187, SEQ ID NO: 188, SEQ ID NO: 189, SEQ ID NO: 190, SEQ ID NO: 191, SEQ ID NO: 192, SEQ ID NO: 193, SEQ ID NO: 194, SEQ ID NO: 195, SEQ ID NO: 196, SEQ ID NO: 197, SEQ ID NO: 198, or SEQ ID NO: 199.

20. The nucleic acid molecule of any one of the preceding claims, wherein the variant C/A junction comprises an amino acid sequence of SEQ ID NO: 52, SEQ ID NO: 53, SEQ ID NO: 54, SEQ ID NO: 55, SEQ ID NO: 56, SEQ ID NO: 57, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, or SEQ ID NO: 136.

21. The nucleic acid molecule of any one of the preceding claims, wherein the variant C/A junction comprises an amino acid sequence of RRKR (SEQ ID NO: 56).

22. The nucleic acid molecule of any one of the preceding claims, wherein the wildtype B-chain is a wildtype human B-chain, a wildtype canine B-chain, or a wildtype feline B-chain.

23. The nucleic acid molecule of any one of the preceding claims, wherein the wildtype B-chain comprises an amino acid sequence of SEQ ID NO: 59 or SEQ ID NO: 66.

24. The nucleic acid molecule of any one of the preceding claims, wherein the wildtype B-chain is a wildtype human B-chain.

25. The nucleic acid molecule of any one of the preceding claims, wherein the wildtype B-chain or full-length variant thereof comprises an amino acid sequence of a or SEQ ID NO: 203.

26. The nucleic acid molecule of any one of the preceding claims, wherein the wildtype C-peptide is a wildtype human C-peptide, a wildtype canine C-peptide, or a wildtype feline C-peptide.

27. The nucleic acid molecule of any one of the preceding claims, wherein the wildtype C-peptide comprises an amino acid sequence of SEQ ID NO: 60, SEQ ID NO: 67, or SEQ ID NO: 70.

28. The nucleic acid molecule of any one of the preceding claims, wherein the wildtype C-peptide is a wildtype human C-peptide.

29. The nucleic acid molecule of any one of the preceding claims, wherein the wildtype C-peptide comprises an amino acid sequence of SEQ ID NO: 60.

30. The nucleic acid molecule of any one of the preceding claims, wherein the wildtype A-chain is a wildtype human A-chain, a wildtype canine A-chain, or a wildtype feline A-chain.

31. The nucleic acid molecule of any one of the preceding claims, wherein the wildtype A-chain comprises an amino acid sequence of SEQ ID NO: 61, SEQ ID NO: 68, or SEQ ID NO: 71.

32. The nucleic acid molecule of any one of the preceding claims, wherein the wildtype A-chain is a wildtype human A-chain.

33. The nucleic acid molecule of any one of the preceding claims, wherein the wildtype A-chain comprises an amino acid sequence of SEQ ID NO: 61.

34. The nucleic acid molecule of any one of the preceding claims, wherein the preproinsulin polypeptide is capable of being processed into a proinsulin polypeptide comprising an amino acid sequence of SEQ ID NO: 2, SEQ ID NO: 72, or SEQ ID NO: 73.

35. The nucleic acid molecule of any one of the preceding claims, wherein the preproinsulin polypeptide is capable of being processed into a proinsulin polypeptide comprising an amino acid sequence of SEQ ID NO: 2.

36. The nucleic acid molecule of any one of the preceding claims, wherein the preproinsulin polypeptide is capable of being processed into a proinsulin polypeptide comprising an amino acid sequence of SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 74, or SEQ ID NO: 75.

37. An nucleic acid molecule comprising a nucleic acid sequence encoding a proinsulin polypeptide comprising an amino acid sequence of SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 74, or SEQ ID NO: 75.

38. The nucleic acid molecule of any one of the preceding claims, wherein the preproinsulin polypeptide is capable of being processed into a proinsulin polypeptide comprising an amino acid sequence of SEQ ID NO: 175.

39. A nucleic acid molecule comprising a nucleic acid sequence encoding a proinsulin polypeptide comprising an amino acid sequence of SEQ ID NO: 175.

40. The nucleic acid molecule of any one of the preceding claims, wherein the N-terminal signal sequence comprises a wildtype N-terminal signal sequence.

41. The nucleic acid molecule of any one of the preceding claims, wherein the N-terminal signal sequence comprises a wildtype human N-terminal signal sequence, a wildtype canine N-terminal signal sequence, or a wildtype feline N-terminal signal sequence.

42. The nucleic acid molecule of any one of the preceding claims, wherein the N-terminal signal sequence comprises an amino acid sequence of SEQ ID NO: 43, SEQ ID NO: 65, or SEQ ID NO: 69.

43. The nucleic acid molecule of any one of the preceding claims, wherein the N-terminal signal sequence comprises a wildtype human N-terminal signal sequence.

44. The nucleic acid molecule of any one of the preceding claims, wherein the N-terminal signal sequence comprises an amino acid sequence of SEQ ID NO: 43.

45. The nucleic acid molecule of any one of the preceding claims, wherein the variant preproinsulin polypeptide comprises an amino acid sequence of SEQ ID NO: 1, SEQ ID NO: 48, or SEQ ID NO: 51.

46. The nucleic acid molecule of any one of the preceding claims, wherein the variant preproinsulin polypeptide comprises an amino acid sequence of SEQ ID NO: 1.

47. The nucleic acid molecule of any one of the preceding claims, wherein the variant preproinsulin polypeptide comprises an amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 47, or SEQ ID NO: 50.

48. A nucleic acid molecule comprising a nucleic acid sequence encoding a variant preproinsulin polypeptide comprising an amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 47, or SEQ ID NO: 50.

49. The nucleic acid molecule of any one of the preceding claims, wherein the variant preproinsulin polypeptide comprises an amino acid sequence of SEQ ID NO: 31.

50. An nucleic acid molecule encoding a variant preproinsulin polypeptide comprising an amino acid sequence of SEQ ID NO: 31.

51. The nucleic acid molecule of any one of the preceding claims comprising a nucleic acid sequence of SEQ ID NO: 77.

52. An nucleic acid molecule comprising a nucleic acid sequence of SEQ ID NO: 77.

53. The nucleic acid molecule of any one of claims 1 to 39, wherein the N-terminal signal sequence comprises a variant N-terminal signal sequence.

54. A variant preproinsulin polypeptide comprising:
a) an N-terminal signal sequence,
b) a wildtype B-chain or full-length variant thereof comprising at least one amino acid substitution,
c) a variant B/C junction comprising at least 4 amino acids selected from histidine, lysine, and arginine,
d) a wildtype C-peptide or full-length variant thereof comprising at least one amino acid substitution,
e) a variant C/A junction comprising at least 4 amino acids selected from histidine, lysine, and arginine, and
f) a wildtype A-chain or full-length variant thereof comprising at least one amino acid substitution,
wherein the variant B/C junction and the variant C/A junction each comprise an enzymatic cleavage site for a target proteolytic enzyme to cleave immediately after the C-terminal amino acid of the junction.

55. The variant preproinsulin polypeptide of claim 54, wherein the enzymatic cleavage site is a subtilisin-like proprotein convertase cleavage site.

56. The variant preproinsulin polypeptide of claim 54 or claim 55, wherein the enzymatic cleavage site is a furin cleavage site.

57. The variant preproinsulin polypeptide of any one of claims 54 to 56, wherein the preproinsulin polypeptide is capable of being processed into a mature wildtype insulin protein or full-length variant thereof comprising at least one amino acid substitution, and a mature wildtype C-peptide or full-length variant thereof comprising at least one amino acid substitution.

58. The variant preproinsulin polypeptide of any one of claims 54 to 57, wherein the preproinsulin polypeptide is capable of being processed by furin and a carboxypeptidase into a mature wildtype insulin protein or full-length variant thereof comprising at least one amino acid substitution, and a mature wildtype C-peptide or full-length variant thereof comprising at least one amino acid substitution.

59. The variant preproinsulin polypeptide of claim 57 or claim 58, wherein the mature wildtype insulin protein is a mature wildtype human insulin protein, mature wildtype canine insulin protein, or mature wildtype feline insulin protein; and wherein the mature wildtype C-peptide is a mature wildtype human C-peptide, mature wildtype canine C-peptide, or mature wildtype feline C-peptide.

60. The variant preproinsulin polypeptide of any one of claims 57 to 59, wherein the mature wildtype insulin protein is a mature wildtype human insulin protein; and wherein the mature wildtype C-peptide is a mature wildtype human C-peptide.

61. The variant preproinsulin polypeptide of any one of claims 54 to 60, wherein the enzymatic cleavage site comprises an amino acid sequence of RX₁X₂R (SEQ ID NO: 45), wherein X₁ is histidine, lysine, or arginine and X₂ is lysine or arginine.

62. The variant preproinsulin polypeptide of any one of claims 54 to 61, wherein each enzymatic cleavage site comprises an amino acid sequence selected from RHKR (SEQ ID NO: 52), RHRR (SEQ ID NO: 53), RKKR (SEQ ID NO: 54), RKRR (SEQ ID NO: 55), RRKR (SEQ ID NO: 56), and RRRR (SEQ ID NO: 57).

63. The variant preproinsulin polypeptide of any one of claims 64 to 62, wherein each enzymatic cleavage site comprises an amino acid sequence of RRKR (SEQ ID NO: 56).

64. The variant preproinsulin polypeptide of any one of claims 54 to 63, wherein the variant B/C junction and the variant C/A junction each comprise 4 to 10 amino acids selected from histidine, lysine, and arginine.

65. The variant preproinsulin polypeptide of any one of claims 54 to 64, wherein the variant B/C junction and the variant C/A junction each comprise 4 to 6 amino acids selected from histidine, lysine, and arginine.

66. The variant preproinsulin polypeptide of any one of claims 54 to 65, wherein the variant B/C junction and the variant C/A junction each comprise 4 amino acids selected from histidine, lysine, and arginine.

67. The variant preproinsulin polypeptide of any one of claims 54 to 66, wherein the variant B/C junction comprises an amino acid sequence of XₙRX₁X₂R (SEQ ID NO: 64), wherein Xₙ is 0, 1, 2, 3, 4, 5, or 6 amino acids each chosen from histidine, lysine, or arginine, X₁ is histidine, lysine, or arginine, and X₂ is lysine or arginine.

68. The variant preproinsulin polypeptide of any one of claims 54 to 67, wherein the variant B/C junction comprises an amino acid sequence of SEQ ID NO: 52, SEQ ID NO: 53, SEQ ID NO: 54, SEQ ID NO: 55, SEQ ID NO: 56, SEQ ID NO: 57, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, or SEQ ID NO: 136.

69. The variant preproinsulin polypeptide of any one of claims 54 to 68, wherein the variant B/C junction comprises an amino acid sequence of RRKR (SEQ ID NO: 56).

70. The variant preproinsulin polypeptide of any one of claims 54 to 69, wherein the variant C/A junction comprises an amino acid sequence of XₙRX₁X₂R (SEQ ID NO: 64), wherein Xₙ is 0, 1, 2, 3, 4, 5, or 6 amino acids each chosen from histidine, lysine, or arginine, X₁ is histidine, lysine, or arginine, and X₂ is lysine or arginine.

71. The variant preproinsulin polypeptide of any one of claims 54 to 70, wherein the variant C/A junction comprises an amino acid sequence of SEQ ID NO: 52, SEQ ID NO: 53, SEQ ID NO: 54, SEQ ID NO: 55, SEQ ID NO: 56, SEQ ID NO: 57, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, or SEQ ID NO: 136.

72. The variant preproinsulin polypeptide of any one of claims 54 to 71, wherein the variant C/A junction comprises an amino acid sequence of RRKR (SEQ ID NO: 56).

73. The variant preproinsulin polypeptide of any one of claims 54 to 72, wherein the wildtype B-chain is a wildtype human B-chain, a wildtype canine B-chain, or a wildtype feline B-chain.

74. The variant preproinsulin polypeptide of any one of claims 54 to 73, wherein the wildtype B-chain comprises an amino acid sequence of SEQ ID NO: 59 or SEQ ID NO: 66.

75. The variant preproinsulin polypeptide of any one of claims 54 to 74, wherein the wildtype B-chain is a wildtype human B-chain.

76. The variant preproinsulin polypeptide of any one of claims 54 to 75, wherein the wildtype B-chain or full-length variant thereof comprises an amino acid sequence of SEQ ID NO: 59 or SEQ ID NO: 203.

77. The variant preproinsulin polypeptide of any one of claims 54 to 76, wherein the wildtype C-peptide is a wildtype human C-peptide, a wildtype canine C-peptide, or a wildtype feline C-peptide.

78. The variant preproinsulin polypeptide of any one of claims 54 to 77, wherein the wildtype C-peptide comprises an amino acid sequence of SEQ ID NO: 60, SEQ ID NO: 67, or SEQ ID NO: 70.

79. The variant preproinsulin polypeptide of any one of claims 54 to 78, wherein the wildtype C-peptide is a wildtype human C-peptide.

80. The variant preproinsulin polypeptide of any one of claims 54 to 79, wherein the wildtype C-peptide comprises an amino acid sequence of SEQ ID NO: 60.

81. The variant preproinsulin polypeptide of any one of claims 54 to 80, wherein the wildtype A-chain is a wildtype human A-chain, a wildtype canine A-chain, or a wildtype feline A-chain.

82. The variant preproinsulin polypeptide of any one of claims 54 to 81, wherein the wildtype A-chain comprises an amino acid sequence of SEQ ID NO: 61, SEQ ID NO: 68, or SEQ ID NO: 71.

83. The variant preproinsulin polypeptide of any one of claims 54 to 82, wherein the wildtype A-chain is a wildtype human A-chain.

84. The variant preproinsulin polypeptide of any one of claims 54 to 83, wherein the wildtype A-chain comprises an amino acid sequence of SEQ ID NO: 61.

85. The variant preproinsulin polypeptide of any one of claims 54 to 84, wherein the preproinsulin polypeptide is capable of being processed into a proinsulin polypeptide comprising an amino acid sequence of SEQ ID NO: 2, SEQ ID NO: 72, or SEQ ID NO: 73.

86. The variant preproinsulin polypeptide of any one of claims 54 to 85, wherein the preproinsulin polypeptide is capable of being processed into a proinsulin polypeptide comprising an amino acid sequence of SEQ ID NO: 2.

87. The variant preproinsulin polypeptide of any one of claims 54 to 86, wherein the preproinsulin polypeptide is capable of being processed into a proinsulin polypeptide comprising an amino acid sequence of SEQ ID NO: 147, SEQ ID NO: 148, SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160, SEQ ID NO: 161, SEQ ID NO: 162, SEQ ID NO: 163, SEQ ID NO: 164, SEQ ID NO: 165, SEQ ID NO: 166, SEQ ID NO: 167, SEQ ID NO: 168, SEQ ID NO: 169, SEQ ID NO: 170, SEQ ID NO: 171, SEQ ID NO: 172, SEQ ID NO: 173, SEQ ID NO: 174, SEQ ID NO: 175, SEQ ID NO: 176, SEQ ID NO: 177, SEQ ID NO: 178, SEQ ID NO: 179, SEQ ID NO: 180, SEQ ID NO: 181, SEQ ID NO: 182, SEQ ID NO: 74, or SEQ ID NO: 75.

88. The variant preproinsulin polypeptide of any one of claims 54 to 87, wherein the preproinsulin polypeptide is capable of being processed into a proinsulin polypeptide comprising an amino acid sequence of SEQ ID NO: 175.

89. The variant preproinsulin polypeptide of any one of claims 54 to 88, wherein the N-terminal signal sequence comprises a wildtype N-terminal signal sequence.

90. The variant preproinsulin polypeptide of any one of claims 54 to 89, wherein the N-terminal signal sequence comprises a wildtype human N-terminal signal sequence, a wildtype canine N-terminal signal sequence, or a wildtype feline N-terminal signal sequence.

91. The variant preproinsulin polypeptide of any one of claims 54 to 90, wherein the N-terminal signal sequence comprises an amino acid sequence of SEQ ID NO: 43, SEQ ID NO: 65, or SEQ ID NO: 69.

92. The variant preproinsulin polypeptide of any one of claims 54 to 91, wherein the N-terminal signal sequence comprises a wildtype human N-terminal signal sequence.

93. The variant preproinsulin polypeptide of any one of claims 54 to 92, wherein the N-terminal signal sequence comprises an amino acid sequence of SEQ ID NO: 43.

94. The variant preproinsulin polypeptide of any one of claims 54 to 93, comprising an amino acid sequence of SEQ ID NO: 1, SEQ ID NO: 48, or SEQ ID NO: 51.

95. The variant preproinsulin polypeptide of any one of claims 54 to 94, comprising an amino acid sequence of SEQ ID NO: 1.

96. The variant preproinsulin polypeptide of any one of claims 54 to 95, comprising an amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 47, or SEQ ID NO: 50.

97. A variant preproinsulin polypeptide comprising an amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 47, or SEQ ID NO: 50.

98. The variant preproinsulin polypeptide of any one of claims 54 to 97, comprising an amino acid sequence of SEQ ID NO: 31.

99. A variant preproinsulin polypeptide comprising an amino acid sequence of SEQ ID NO: 31.

100. The variant preproinsulin polypeptide of any one of claims 54 to 88, wherein the N-terminal signal sequence comprises a variant N-terminal signal sequence.

101. An nucleic acid molecule comprising a nucleic acid sequence encoding the variant preproinsulin polypeptide of any one of claims 54 to 100.

102. The nucleic acid molecule of any one of claims 1 to 53 or claim 101, further comprising a promoter operatively linked to the nucleic acid sequence encoding the variant preproinsulin polypeptide.

103. The nucleic acid molecule of claim 102, wherein the promoter is a constitutive promoter.

104. The nucleic acid molecule of claim 102, wherein the promoter is a regulated promoter.

105. The nucleic acid molecule of claim 102, wherein the promoter is an albumin promoter.

106. The nucleic acid molecule of any one of claims 1 to 53 or any one of claims 101 to 105, further comprising at least one GIRE element.

107. A vector comprising the nucleic acid of any one of claims 1 to 53 or any one of claims 101 to 106.

108. A vector comprising a nucleic acid comprising the nucleic acid sequence of SEQ ID NO: 76 or SEQ ID NO: 77.

109. The vector of claim 107 or claim 108, wherein the vector is a viral vector.

110. The vector of any one of claims 107 to 109, wherein the vector is an adeno-associated virus (AAV) vector, an adenovirus vector, a retrovirus vector, a herpesvirus vector, a pox virus vector, a synthetic mRNA, or a self-replicating RNA.

111. The vector of any one of claims 107 to 110, wherein the vector is an adeno-associated virus (AAV) vector.

112. The vector of any one of claims 107 to 111, wherein the vector is a self-complementary adeno-associated virus (scAAV) vector.

113. The vector of any one of claims 107 to 112, wherein the vector is an adeno-associated virus (AAV) vector having a capsid serotype selected from the group consisting of AAV1, AAV2, AAV3, AAV4, AAV5, AAV6, AAV7, AAV8, AAV9, AAV10, AAV11, AAV12, AAV13, and any variant thereof.

114. The vector of claim 113, wherein the AAV vector has a capsid serotype of AAV8.

115. A cultured host cell comprising the nucleic acid of any one of claims 1 to 53 or any one of claims 101 to 106, the variant preproinsulin polypeptide of any one of claims 54 to 100, or the vector of any one of claims 107 to 114.

116. A pharmaceutical composition comprising the nucleic acid of any one of claims 1 to 53 or any one of claims 101 to 106, the variant preproinsulin polypeptide of any one of claims 54 to 100, the vector of any one of claims 107 to 114, or the cultured host cell of claim 115 and a pharmaceutically acceptable carrier.

117. A method of treating a subject with diabetes comprising administering to the subject the nucleic acid of any one of claims 1 to 53 or any one of claims 101 to 106, the variant preproinsulin polypeptide of any one of claims 54 to 100, the vector of any one of claims 107 to 114, the cultured host cell of claim 115, or the pharmaceutical composition of claim 116.

118. The method of claim 117, wherein the nucleic acid, the variant preproinsulin polypeptide, the vector, the cultured host cell, and/or the pharmaceutical composition is administered to the subject via intravenous injection, arterial injection, intramuscular injection, intradermal injection, intraperitoneal injection, and/or subcutaneous injection.

119. The method of claim 117 or claim 118, wherein the vector is administered to the subject at a dose of about 1×10¹⁰ vector genomes per kilogram, about 1×10¹¹ vector genomes per kilogram, about 1×10¹² vector genomes per kilogram, about 1×10¹³ vector genomes per kilogram, about 1×10¹⁰ to about 1×10¹³ vector genomes per kilogram, about 1×10¹⁰ to about 1×10¹² vector genomes per kilogram, about 1×10¹¹ to about 1×10¹² vector genomes per kilogram, about 1×10¹⁰ to about 1×10¹¹ vector genomes per kilogram or about 1×10¹² to about 1×10¹³ vector genomes per kilogram.

120. The method of any one of claims 117 to 119, wherein the subject is a human, a dog, or a cat.

121. The method of any one of claims 117 to 120, wherein the subject is a human subject.

122. The method of claim 117, wherein the diabetes in Type 1 diabetes.

123. A method of producing insulin in a cell, the method comprising transducing, transfecting, or transforming the cell with the nucleic acid of any one of claims 1 to 53 or any one of claims 101 to 106 or the vector of any one of claims 107 to 114.

124. The method of claim 123, wherein the cell is exposed to the nucleic acid or the vector ex vivo.

125. The method of claim 123, wherein the cell is exposed to the nucleic acid or the vector in vivo.

126. The method of any one of claims 123 to 125, wherein the cell is a human cell, a canine cell, or a feline cell.

127. The method of any one of claims 123 to 126, wherein the cell is a liver cell or a muscle cell.


SEQUENCE LISTING (207 sequences)

SEQ ID NO: 1 - protein, 126 aa, synthetic construct
  REGION 1..126: note = Synthetic: Exemplary Variant Human Preproinsulin; Consensus based on ENDSULIN101 Preproinsulin
  VARIANT 55..60: note = X may or may not be present; if present, X is H, K or R
  VARIANT 62: note = X is H, K, or R
  VARIANT 63: note = X is R or K
  VARIANT 96..101: note = X may or may not be present; if present, X is H, K or R
  VARIANT 103: note = X is H, K, or R
  VARIANT 104: note = X is R or K
MALWMRLLPLLALLALWGPDPAAAFVNQHLCGSHLVEALYLVCGERGFFYTPKTXXXXXXRXXREAEDLQVGQVELGGGPGAGSLQPLALEGSLQXXXXXXRXXRGIVEQCCTSICSLYQLENYCN

SEQ ID NO: 2 - protein, 102 aa, synthetic construct
  REGION 1..102: note = Synthetic: Exemplary Variant Human Proinsulin ; Consensus based on ENDSULIN101 Proinsulin
  VARIANT 31..36: note = X may or may not be present; if present, X is H, K or R
  VARIANT 38: note = X is H, K, or R
  VARIANT 39: note = X is R or K
  VARIANT 72..77: note = X may or may not be present; if present, X is H, K or R
  VARIANT 79: note = X is H, K, or R
  VARIANT 80: note = X is R or K
FVNQHLCGSHLVEALYLVCGERGFFYTPKTXXXXXXRXXREAEDLQVGQVELGGGPGAGSLQPLALEGSLQXXXXXXRXXRGIVEQCCTSICSLYQLENYCN

SEQ ID NO: 3 - protein, 114 aa, synthetic construct
  REGION 1..114: note = Synthetic: Exemplary preproinsulin 1
MALWMRLLPLLALLALWGPDPAAAFVNQHLCGSHLVEALYLVCGERGFFYTPKTRHKREAEDLQVGQVELGGGPGAGSLQPLALEGSLQRHKRGIVEQCCTSICSLYQLENYCN

SEQ ID NO: 4 - protein, 114 aa, synthetic construct
  REGION 1..114: note = Synthetic: Exemplary preproinsulin 2
MALWMRLLPLLALLALWGPDPAAAFVNQHLCGSHLVEALYLVCGERGFFYTPKTRHKREAEDLQVGQVELGGGPGAGSLQPLALEGSLQRHRRGIVEQCCTSICSLYQLENYCN

SEQ ID NO: 5 - protein, 114 aa, synthetic construct
  REGION 1..114: note = Synthetic: Exemplary preproinsulin 3
MALWMRLLPLLALLALWGPDPAAAFVNQHLCGSHLVEALYLVCGERGFFYTPKTRHKREAEDLQVGQVELGGGPGAGSLQPLALEGSLQRKKRGIVEQCCTSICSLYQLENYCN

SEQ ID NO: 6 - protein, 114 aa, synthetic construct
  REGION 1..114: note = Synthetic: Exemplary preproinsulin 4
MALWMRLLPLLALLALWGPDPAAAFVNQHLCGSHLVEALYLVCGERGFFYTPKTRHKREAEDLQVGQVELGGGPGAGSLQPLALEGSLQRKRRGIVEQCCTSICSLYQLENYCN

SEQ ID NO: 7 - protein, 114 aa, synthetic construct
  REGION 1..114: note = Synthetic: Exemplary preproinsulin 5
MALWMRLLPLLALLALWGPDPAAAFVNQHLCGSHLVEALYLVCGERGFFYTPKTRHKREAEDLQVGQVELGGGPGAGSLQPLALEGSLQRRKRGIVEQCCTSICSLYQLENYCN

SEQ ID NO: 8 - protein, 114 aa, synthetic construct
  REGION 1..114: note = Synthetic: Exemplary preproinsulin 6
MALWMRLLPLLALLALWGPDPAAAFVNQHLCGSHLVEALYLVCGERGFFYTPKTRHKREAEDLQVGQVELGGGPGAGSLQPLALEGSLQRRRRGIVEQCCTSICSLYQLENYCN

SEQ ID NO: 9 - protein, 114 aa, synthetic construct
  REGION 1..114: note = Synthetic: Exemplary preproinsulin 7
MALWMRLLPLLALLALWGPDPAAAFVNQHLCGSHLVEALYLVCGERGFFYTPKTRHRREAEDLQVGQVELGGGPGAGSLQPLALEGSLQRHKRGIVEQCCTSICSLYQLENYCN

SEQ ID NO: 10 - protein, 114 aa, synthetic construct
  REGION 1..114: note = Synthetic: Exemplary preproinsulin 8
MALWMRLLPLLALLALWGPDPAAAFVNQHLCGSHLVEALYLVCGERGFFYTPKTRHRREAEDLQVGQVELGGGPGAGSLQPLALEGSLQRHRRGIVEQCCTSICSLYQLENYCN

SEQ ID NO: 11 - protein, 114 aa, synthetic construct
  REGION 1..114: note = Synthetic: Exemplary preproinsulin 9
MALWMRLLPLLALLALWGPDPAAAFVNQHLCGSHLVEALYLVCGERGFFYTPKTRHRREAEDLQVGQVELGGGPGAGSLQPLALEGSLQRKKRGIVEQCCTSICSLYQLENYCN

SEQ ID NO: 12 - protein, 114 aa, synthetic construct
  REGION 1..114: note = Synthetic: Exemplary preproinsulin 10
MALWMRLLPLLALLALWGPDPAAAFVNQHLCGSHLVEALYLVCGERGFFYTPKTRHRREAEDLQVGQVELGGGPGAGSLQPLALEGSLQRKRRGIVEQCCTSICSLYQLENYCN

SEQ ID NO: 13 - protein, 114 aa, synthetic construct
  REGION 1..114: note = Synthetic: Exemplary preproinsulin 11
MALWMRLLPLLALLALWGPDPAAAFVNQHLCGSHLVEALYLVCGERGFFYTPKTRHRREAEDLQVGQVELGGGPGAGSLQPLALEGSLQRRKRGIVEQCCTSICSLYQLENYCN

SEQ ID NO: 14 - protein, 114 aa, synthetic construct
  REGION 1..114: note = Synthetic: Exemplary preproinsulin 12
MALWMRLLPLLALLALWGPDPAAAFVNQHLCGSHLVEALYLVCGERGFFYTPKTRHRREAEDLQVGQVELGGGPGAGSLQPLALEGSLQRRRRGIVEQCCTSICSLYQLENYCN

SEQ ID NO: 15 - protein, 114 aa, synthetic construct
  REGION 1..114: note = Synthetic: Exemplary preproinsulin 13
MALWMRLLPLLALLALWGPDPAAAFVNQHLCGSHLVEALYLVCGERGFFYTPKTRKKREAEDLQVGQVELGGGPGAGSLQPLALEGSLQRHKRGIVEQCCTSICSLYQLENYCN

SEQ ID NO: 16 - protein, 114 aa, synthetic construct
  REGION 1..114: note = Synthetic: Exemplary preproinsulin 14
MALWMRLLPLLALLALWGPDPAAAFVNQHLCGSHLVEALYLVCGERGFFYTPKTRKKREAEDLQVGQVELGGGPGAGSLQPLALEGSLQRHRRGIVEQCCTSICSLYQLENYCN

SEQ ID NO: 17 - protein, 114 aa, synthetic construct
  REGION 1..114: note = Synthetic: Exemplary preproinsulin 15
MALWMRLLPLLALLALWGPDPAAAFVNQHLCGSHLVEALYLVCGERGFFYTPKTRKKREAEDLQVGQVELGGGPGAGSLQPLALEGSLQRKKRGIVEQCCTSICSLYQLENYCN

SEQ ID NO: 18 - protein, 114 aa, synthetic construct
  REGION 1..114: note = Synthetic: Exemplary preproinsulin 16
MALWMRLLPLLALLALWGPDPAAAFVNQHLCGSHLVEALYLVCGERGFFYTPKTRKKREAEDLQVGQVELGGGPGAGSLQPLALEGSLQRKRRGIVEQCCTSICSLYQLENYCN

SEQ ID NO: 19 - protein, 114 aa, synthetic construct
  REGION 1..114: note = Synthetic: Exemplary preproinsulin 17
MALWMRLLPLLALLALWGPDPAAAFVNQHLCGSHLVEALYLVCGERGFFYTPKTRKKREAEDLQVGQVELGGGPGAGSLQPLALEGSLQRRKRGIVEQCCTSICSLYQLENYCN

SEQ ID NO: 20 - protein, 114 aa, synthetic construct
  REGION 1..114: note = Synthetic: Exemplary preproinsulin 18
MALWMRLLPLLALLALWGPDPAAAFVNQHLCGSHLVEALYLVCGERGFFYTPKTRKKREAEDLQVGQVELGGGPGAGSLQPLALEGSLQRRRRGIVEQCCTSICSLYQLENYCN

SEQ ID NO: 21 - protein, 114 aa, synthetic construct
  REGION 1..114: note = Synthetic: Exemplary preproinsulin 19
MALWMRLLPLLALLALWGPDPAAAFVNQHLCGSHLVEALYLVCGERGFFYTPKTRKRREAEDLQVGQVELGGGPGAGSLQPLALEGSLQRHKRGIVEQCCTSICSLYQLENYCN

SEQ ID NO: 22 - protein, 114 aa, synthetic construct
  REGION 1..114: note = Synthetic: Exemplary preproinsulin 20
MALWMRLLPLLALLALWGPDPAAAFVNQHLCGSHLVEALYLVCGERGFFYTPKTRKRREAEDLQVGQVELGGGPGAGSLQPLALEGSLQRHRRGIVEQCCTSICSLYQLENYCN

SEQ ID NO: 23 - protein, 114 aa, synthetic construct
  REGION 1..114: note = Synthetic: Exemplary preproinsulin 21
MALWMRLLPLLALLALWGPDPAAAFVNQHLCGSHLVEALYLVCGERGFFYTPKTRKRREAEDLQVGQVELGGGPGAGSLQPLALEGSLQRKKRGIVEQCCTSICSLYQLENYCN

SEQ ID NO: 24 - protein, 114 aa, synthetic construct
  REGION 1..114: note = Synthetic: Exemplary preproinsulin 22
MALWMRLLPLLALLALWGPDPAAAFVNQHLCGSHLVEALYLVCGERGFFYTPKTRKRREAEDLQVGQVELGGGPGAGSLQPLALEGSLQRKRRGIVEQCCTSICSLYQLENYCN

SEQ ID NO: 25 - protein, 114 aa, synthetic construct
  REGION 1..114: note = Synthetic: Exemplary preproinsulin 23
MALWMRLLPLLALLALWGPDPAAAFVNQHLCGSHLVEALYLVCGERGFFYTPKTRKRREAEDLQVGQVELGGGPGAGSLQPLALEGSLQRRKRGIVEQCCTSICSLYQLENYCN

SEQ ID NO: 26 - protein, 114 aa, synthetic construct
  REGION 1..114: note = Synthetic: Exemplary preproinsulin 24
MALWMRLLPLLALLALWGPDPAAAFVNQHLCGSHLVEALYLVCGERGFFYTPKTRKRREAEDLQVGQVELGGGPGAGSLQPLALEGSLQRRRRGIVEQCCTSICSLYQLENYCN

SEQ ID NO: 27 - protein, 114 aa, synthetic construct
  REGION 1..114: note = Synthetic: Exemplary preproinsulin 25
MALWMRLLPLLALLALWGPDPAAAFVNQHLCGSHLVEALYLVCGERGFFYTPKTRRKREAEDLQVGQVELGGGPGAGSLQPLALEGSLQRHKRGIVEQCCTSICSLYQLENYCN

SEQ ID NO: 28 - protein, 114 aa, synthetic construct
  REGION 1..114: note = Synthetic: Exemplary preproinsulin 26
MALWMRLLPLLALLALWGPDPAAAFVNQHLCGSHLVEALYLVCGERGFFYTPKTRRKREAEDLQVGQVELGGGPGAGSLQPLALEGSLQRHRRGIVEQCCTSICSLYQLENYCN

SEQ ID NO: 29 - protein, 114 aa, synthetic construct
  REGION 1..114: note = Synthetic: Exemplary preproinsulin 27
MALWMRLLPLLALLALWGPDPAAAFVNQHLCGSHLVEALYLVCGERGFFYTPKTRRKREAEDLQVGQVELGGGPGAGSLQPLALEGSLQRKKRGIVEQCCTSICSLYQLENYCN

SEQ ID NO: 30 - protein, 114 aa, synthetic construct
  REGION 1..114: note = Synthetic: Exemplary preproinsulin 28
MALWMRLLPLLALLALWGPDPAAAFVNQHLCGSHLVEALYLVCGERGFFYTPKTRRKREAEDLQVGQVELGGGPGAGSLQPLALEGSLQRKRRGIVEQCCTSICSLYQLENYCN

SEQ ID NO: 31 - protein, 114 aa, synthetic construct
  REGION 1..114: note = Synthetic: Exemplary preproinsulin 29 ENDSULIN101-Human sequence
MALWMRLLPLLALLALWGPDPAAAFVNQHLCGSHLVEALYLVCGERGFFYTPKTRRKREAEDLQVGQVELGGGPGAGSLQPLALEGSLQRRKRGIVEQCCTSICSLYQLENYCN

SEQ ID NO: 32 - protein, 114 aa, synthetic construct
  REGION 1..114: note = Synthetic: Exemplary preproinsulin 30
MALWMRLLPLLALLALWGPDPAAAFVNQHLCGSHLVEALYLVCGERGFFYTPKTRRKREAEDLQVGQVELGGGPGAGSLQPLALEGSLQRRRRGIVEQCCTSICSLYQLENYCN

SEQ ID NO: 33 - protein, 114 aa, synthetic construct
  REGION 1..114: note = Synthetic: Exemplary preproinsulin 31
MALWMRLLPLLALLALWGPDPAAAFVNQHLCGSHLVEALYLVCGERGFFYTPKTRRRREAEDLQVGQVELGGGPGAGSLQPLALEGSLQRHKRGIVEQCCTSICSLYQLENYCN

SEQ ID NO: 34 - protein, 114 aa, synthetic construct
  REGION 1..114: note = Synthetic: Exemplary preproinsulin 32
MALWMRLLPLLALLALWGPDPAAAFVNQHLCGSHLVEALYLVCGERGFFYTPKTRRRREAEDLQVGQVELGGGPGAGSLQPLALEGSLQRHRRGIVEQCCTSICSLYQLENYCN

SEQ ID NO: 35 - protein, 114 aa, synthetic construct
  REGION 1..114: note = Synthetic: Exemplary preproinsulin 33
MALWMRLLPLLALLALWGPDPAAAFVNQHLCGSHLVEALYLVCGERGFFYTPKTRRRREAEDLQVGQVELGGGPGAGSLQPLALEGSLQRKKRGIVEQCCTSICSLYQLENYCN

SEQ ID NO: 36 - protein, 114 aa, synthetic construct
  REGION 1..114: note = Synthetic: Exemplary preproinsulin 34
MALWMRLLPLLALLALWGPDPAAAFVNQHLCGSHLVEALYLVCGERGFFYTPKTRRRREAEDLQVGQVELGGGPGAGSLQPLALEGSLQRKRRGIVEQCCTSICSLYQLENYCN

SEQ ID NO: 37 - protein, 114 aa, synthetic construct
  REGION 1..114: note = Synthetic: Exemplary preproinsulin 35
MALWMRLLPLLALLALWGPDPAAAFVNQHLCGSHLVEALYLVCGERGFFYTPKTRRRREAEDLQVGQVELGGGPGAGSLQPLALEGSLQRRKRGIVEQCCTSICSLYQLENYCN

SEQ ID NO: 38 - protein, 114 aa, synthetic construct
  REGION 1..114: note = Synthetic: Exemplary preproinsulin 36
MALWMRLLPLLALLALWGPDPAAAFVNQHLCGSHLVEALYLVCGERGFFYTPKTRRRREAEDLQVGQVELGGGPGAGSLQPLALEGSLQRRRRGIVEQCCTSICSLYQLENYCN

SEQ ID NO: 39 - protein, 110 aa, Homo sapiens
  REGION 1..110: note = misc_feature - Human Wildtype Preproinsulin
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 40 - protein, 110 aa, synthetic construct
  REGION 1..110: note = Synthetic: 1994 Groskreutz-Human Preproinsulin
MALWMRLLPLLALLALWGPDPAAAFVNQHLCGSHLVEALYLVCGERGFFYTPRTKREAEDLQVGQVELGGGPGAGSLQPLALEGSRQKRGIVEQCCTSICSLYQLENYCN

SEQ ID NO: 41 - protein, 110 aa, synthetic construct
  REGION 1..110: note = Synthetic: 1992 Yanagita-Human Preproinsulin
MALWMRLLPLLALLALWGPDPAAAFVNQHLCGSHLVEALYLVCGERGFFYTPKTRRKREDLQVGQVELGGGPGAGSLQPLALEGSRRKRGIVEQCCTSICSLYQLENYCN

SEQ ID NO: 42 - protein, 110 aa, synthetic construct
  REGION 1..110: note = Synthetic: 1994 Groskreutz-Rat preproinsulin
MALWMRFLPLLALLVLWEPKPAQAFVKQHLCGPHLVEALYLVCGERGFFYTPRTKREVEDPQVPQLELGGGPEAGDLQTLALEVARQKRGIVDQCCTSICSLYQLENYCN

SEQ ID NO: 43 - protein, 24 aa, Homo sapiens
  REGION 1..24: note = misc_feature - Human Wildtype N-terminal signal sequence (24 AA)
MALWMRLLPLLALLALWGPDPAAA

SEQ ID NO: 44-45 - skipped (2 entries)

SEQ ID NO: 46 - protein, 110 aa, unidentified
  REGION 1..110: note = Canine Wildtype Preproinsulin
MALWMRLLPLLALLALWAPAPTRAFVNQHLCGSHLVEALYLVCGERGFFYTPKARREVEDLQVRDVELAGAPGEGGLQPLALEGALQKRGIVEQCCTSICSLYQLENYCN

SEQ ID NO: 47 - protein, 114 aa, synthetic construct
  REGION 1..114: note = Synthetic: Exemplary variant canine preproinsulin; ENDSULIN101-Canine Preproinsulin
MALWMRLLPLLALLALWAPAPTRAFVNQHLCGSHLVEALYLVCGERGFFYTPKARRKREVEDLQVRDVELAGAPGEGGLQPLALEGALQRRKRGIVEQCCTSICSLYQLENYCN

SEQ ID NO: 48 - protein, 126 aa, synthetic construct
  REGION 1..126: note = Synthetic: Exemplary Variant Canine Preproinsulin; Consensus based on ENDSULIN101-Canine
  VARIANT 55..60: note = X may or may not be present; if present, X is H, K or R
  VARIANT 62: note = X is H, K, or R
  VARIANT 63: note = X is R or K
  VARIANT 96..101: note = X may or may not be present; if present, X is H, K or R
  VARIANT 103: note = X is H, K, or R
  VARIANT 104: note = X is R or K
MALWMRLLPLLALLALWAPAPTRAFVNQHLCGSHLVEALYLVCGERGFFYTPKAXXXXXXRXXREVEDLQVRDVELAGAPGEGGLQPLALEGALQXXXXXXRXXRGIVEQCCTSICSLYQLENYCN

SEQ ID NO: 49 - protein, 110 aa, unidentified
  REGION 1..110: note = Feline Wildtype Preproinsulin
MAPWTRLLPLLALLSLWIPAPTRAFVNQHLCGSHLVEALYLVCGERGFFYTPKARREAEDLQGKDAELGEAPGAGGLQPSALEAPLQKRGIVEQCCASVCSLYQLEHYCN

SEQ ID NO: 50 - protein, 114 aa, synthetic construct
  REGION 1..114: note = Synthetic: Exemplary variant feline preproinsulin; ENDSULIN101-Feline Preproinsulin
MAPWTRLLPLLALLSLWIPAPTRAFVNQHLCGSHLVEALYLVCGERGFFYTPKARRKREAEDLQGKDAELGEAPGAGGLQPSALEAPLQRRKRGIVEQCCASVCSLYQLEHYCN

SEQ ID NO: 51 - protein, 126 aa, synthetic construct
  REGION 1..126: note = Synthetic: Exemplary Variant Feline Preproinsulin; Consensus based on ENDSULIN101-Feline
  VARIANT 55..60: note = X may or may not be present; if present, X is H, K or R
  VARIANT 62: note = X is H, K, or R
  VARIANT 63: note = X is R or K
  VARIANT 96..101: note = X may or may not be present; if present, X is H, K or R
  VARIANT 103: note = X is H, K, or R
  VARIANT 104: note = X is R or K
MAPWTRLLPLLALLSLWIPAPTRAFVNQHLCGSHLVEALYLVCGERGFFYTPKAXXXXXXRXXREAEDLQGKDAELGEAPGAGGLQPSALEAPLQXXXXXXRXXRGIVEQCCASVCSLYQLEHYCN

SEQ ID NO: 52 - protein, 4 aa, synthetic construct
  REGION 1..4: note = Synthetic: Exemplary Variant Junction Sequence 1
RHKR

SEQ ID NO: 53 - protein, 4 aa, synthetic construct
  REGION 1..4: note = Synthetic: Exemplary Variant Junction Sequence 2
RHRR

SEQ ID NO: 54 - protein, 4 aa, synthetic construct
  REGION 1..4: note = Synthetic: Exemplary Variant Junction Sequence 3
RKKR

SEQ ID NO: 55 - protein, 4 aa, synthetic construct
  REGION 1..4: note = Synthetic: Exemplary Variant Junction Sequence 4
RKRR

SEQ ID NO: 56 - protein, 4 aa, synthetic construct
  REGION 1..4: note = Synthetic: Exemplary Variant Junction Sequence 5
RRKR

SEQ ID NO: 57 - protein, 4 aa, synthetic construct
  REGION 1..4: note = Synthetic: Exemplary Variant Junction Sequence 6
RRRR

SEQ ID NO: 58 - skipped

SEQ ID NO: 59 - protein, 30 aa, Homo sapiens
  REGION 1..30: note = misc_feature - Human Wildtype B-chain
FVNQHLCGSHLVEALYLVCGERGFFYTPKT

SEQ ID NO: 60 - protein, 31 aa, Homo sapiens
  REGION 1..31: note = misc_feature - Human Wildtype C-peptide
EAEDLQVGQVELGGGPGAGSLQPLALEGSLQ

SEQ ID NO: 61 - protein, 21 aa, Homo sapiens
  REGION 1..21: note = misc_feature - Human Wildtype A-chain
GIVEQCCTSICSLYQLENYCN

SEQ ID NO: 62-64 - skipped (3 entries)

SEQ ID NO: 65 - protein, 24 aa, unidentified
  REGION 1..24: note = Canine ; Wildtype N-terminal signal sequence
MALWMRLLPLLALLALWAPAPTRA

SEQ ID NO: 66 - protein, 30 aa, unidentified
  REGION 1..30: note = Canine and Feline; Wildtype B-Chain
FVNQHLCGSHLVEALYLVCGERGFFYTPKA

SEQ ID NO: 67 - protein, 31 aa, unidentified
  REGION 1..31: note = Canine ; Wildtype C-Peptide
EVEDLQVRDVELAGAPGEGGLQPLALEGALQ

SEQ ID NO: 68 - protein, 21 aa, unidentified
  REGION 1..21: note = Canine ; Wildtype A-Chain
GIVEQCCTSICSLYQLENYCN

SEQ ID NO: 69 - protein, 24 aa, unidentified
  REGION 1..24: note = Feline ; N-terminal signal sequence
MAPWTRLLPLLALLSLWIPAPTRA

SEQ ID NO: 70 - protein, 31 aa, unidentified
  REGION 1..31: note = Feline ; Wildtype C-Peptide
EAEDLQGKDAELGEAPGAGGLQPSALEAPLQ

SEQ ID NO: 71 - protein, 21 aa, unidentified
  REGION 1..21: note = Feline ; Wildtype A-Chain
GIVEQCCASVCSLYQLEHYCN

SEQ ID NO: 72 - protein, 102 aa, synthetic construct
  REGION 1..102: note = Synthetic: Variant Canine Proinsulin; Consensus based on ENDSULIN101-Canine
  VARIANT 31..36: note = X may or may not be present; if present, X is H, K or R
  VARIANT 38: note = X is H, K, or R
  VARIANT 39: note = X is R or K
  VARIANT 72..77: note = X may or may not be present; if present, X is H, K or R
  VARIANT 79: note = X is H, K, or R
  VARIANT 80: note = X is R or K
FVNQHLCGSHLVEALYLVCGERGFFYTPKAXXXXXXRXXREVEDLQVRDVELAGAPGEGGLQPLALEGALQXXXXXXRXXRGIVEQCCTSICSLYQLENYCN

SEQ ID NO: 73 - protein, 102 aa, synthetic construct
  REGION 1..102: note = Synthetic: Variant Feline Proinsulin; Consensus based on ENDSULIN101-Feline
  VARIANT 31..36: note = X may or may not be present; if present, X is H, K or R
  VARIANT 38: note = X is H, K, or R
  VARIANT 39: note = X is R or K
  VARIANT 72..77: note = X may or may not be present; if present, X is H, K or R
  VARIANT 79: note = X is H, K, or R
  VARIANT 80: note = X is R or K
FVNQHLCGSHLVEALYLVCGERGFFYTPKAXXXXXXRXXREAEDLQGKDAELGEAPGAGGLQPSALEAPLQXXXXXXRXXRGIVEQCCASVCSLYQLEHYCN

SEQ ID NO: 74 - protein, 90 aa, synthetic construct
  REGION 1..90: note = Synthetic: ENDSULIN101-Canine Proinsulin
FVNQHLCGSHLVEALYLVCGERGFFYTPKARRKREVEDLQVRDVELAGAPGEGGLQPLALEGALQRRKRGIVEQCCTSICSLYQLENYCN

SEQ ID NO: 75 - protein, 90 aa, synthetic construct
  REGION 1..90: note = Synthetic: ENDSULIN101-Feline Proinsulin
FVNQHLCGSHLVEALYLVCGERGFFYTPKARRKREAEDLQGKDAELGEAPGAGGLQPSALEAPLQRRKRGIVEQCCASVCSLYQLEHYCN

SEQ ID NO: 76 - DNA, 2355 bp, synthetic construct
  misc_feature 1..2355: note = Synthetic: Human ENDSULIN101 ITR to ITR
  source 1..2355: mol_type = other DNA
cagctgcgcgctcgctcgctcactgaggccgcccgggcaaagcccgggcgtcgggcgacctttggtcgcccggcctcagtgagcgagcgagcgcgcagagagggagtggggttcctgctattcccatatgattctagagctcgtcgacccgcccccttcaccaagatctttttgatggcagagttcagtttaccgggtcacattgtacctgggaagattcaaggatttatggaaaaagtcaacaacaggagtcagagcagccggaaaagcatggactctgtacttaggactgcgctttgagcaatggcacagcaagctttaaccctgtttgcagtcagcacacaaactgtggttcaaagctccactttatctcttcttgtggaattcagatatcagatcagtttaaaccttgcggccgccagttctcacgtggtggccacgtgcttgggcactctagtgctcaaatgggagacaaagagattaagctcttatgtaaaatttgctgttttacataactttaatgaatggacaaagtcttgtgcatgggggtgggggtggggttagaggggaacagctccagatggcaaacatacgcaagggatttagtcaaacaactttttggcaaagatggtatgattttgtaatggggtaggaaccaatgaaatgcgaggtaagtatggttaataatctacagttattggttaaagaagtatattagagcgagtctttctgcacacagatcaccttcctatcaaccccactagcctctggcaaaggtaccagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccggaagagagaggcagaggacctgcaggtggggcaggtggagctgggcgggggccctggtgcaggcagcctgcagcccttggccctggaggggtccctgcagagacggaagcgtggcattgtggaacaatgctgtaccagcatctgctccctctaccagctggagaactactgcaactagacgcagcctgcaggcagcgtcgagtaacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctatcgattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaagttatggattataaacattcaaaataatattttgacattatgataattctgaataaaagaacaaaaaccatggtataggtaaggaatataaaacatggcttttaccttagaaaaaacaattctaaaattcatatgattctagaactagtggggtaccgaattcagatcgagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcg

SEQ ID NO: 77 - DNA, 345 bp, synthetic construct
  misc_feature 1..345: note = Synthetic: Human ENDSULIN101 Encoding Sequence
  source 1..345: mol_type = other DNA
atggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccggaagagagaggcagaggacctgcaggtggggcaggtggagctgggcgggggccctggtgcaggcagcctgcagcccttggccctggaggggtccctgcagagacggaagcgtggcattgtggaacaatgctgtaccagcatctgctccctctaccagctggagaactactgcaactag

SEQ ID NO: 78 - protein, 5 aa, synthetic construct
  REGION 1..5: note = Synthetic: Exemplary Variant Junction Sequence 7
HRHKR

SEQ ID NO: 79 - protein, 5 aa, synthetic construct
  REGION 1..5: note = Synthetic: Exemplary Variant Junction Sequence 8
HRHRR

SEQ ID NO: 80 - protein, 5 aa, synthetic construct
  REGION 1..5: note = Synthetic: Exemplary Variant Junction Sequence 9
HRKKR

SEQ ID NO: 81 - protein, 5 aa, synthetic construct
  REGION 1..5: note = Synthetic: Exemplary Variant Junction Sequence 10
HRKRR

SEQ ID NO: 82 - protein, 5 aa, synthetic construct
  REGION 1..5: note = Synthetic: Exemplary Variant Junction Sequence 11
HRRKR

SEQ ID NO: 83 - protein, 5 aa, synthetic construct
  REGION 1..5: note = Synthetic: Exemplary Variant Junction Sequence 12
HRRRR

SEQ ID NO: 84 - protein, 5 aa, synthetic construct
  REGION 1..5: note = Synthetic: Exemplary Variant Junction Sequence 13
KRHKR

SEQ ID NO: 85 - protein, 5 aa, synthetic construct
  REGION 1..5: note = Synthetic: Exemplary Variant Junction Sequence 14
KRHRR

SEQ ID NO: 86 - protein, 5 aa, synthetic construct
  REGION 1..5: note = Synthetic: Exemplary Variant Junction Sequence 15
KRKKR

SEQ ID NO: 87 - protein, 5 aa, synthetic construct
  REGION 1..5: note = Synthetic: Exemplary Variant Junction Sequence 16
KRKRR

SEQ ID NO: 88 - protein, 5 aa, synthetic construct
  REGION 1..5: note = Synthetic: Exemplary Variant Junction Sequence 17
KRRKR

SEQ ID NO: 89 - protein, 5 aa, synthetic construct
  REGION 1..5: note = Synthetic: Exemplary Variant Junction Sequence 18
KRRRR

SEQ ID NO: 90 - protein, 5 aa, synthetic construct
  REGION 1..5: note = Synthetic: Exemplary Variant Junction Sequence 19
RRHKR

SEQ ID NO: 91 - protein, 5 aa, synthetic construct
  REGION 1..5: note = Synthetic: Exemplary Variant Junction Sequence 20
RRHRR

SEQ ID NO: 92 - protein, 5 aa, synthetic construct
  REGION 1..5: note = Synthetic: Exemplary Variant Junction Sequence 21
RRKKR

SEQ ID NO: 93 - protein, 5 aa, synthetic construct
  REGION 1..5: note = Synthetic: Exemplary Variant Junction Sequence 22
RRRKR

SEQ ID NO: 94 - protein, 6 aa, synthetic construct
  REGION 1..6: note = Synthetic: Exemplary Variant Junction Sequence 25
HHRHKR

SEQ ID NO: 95 - protein, 6 aa, synthetic construct
  REGION 1..6: note = Synthetic: Exemplary Variant Junction Sequence 26
HHRHRR

SEQ ID NO: 96 - protein, 6 aa, synthetic construct
  REGION 1..6: note = Synthetic: Exemplary Variant Junction Sequence 27
HHRKKR

SEQ ID NO: 97 - protein, 6 aa, synthetic construct
  REGION 1..6: note = Synthetic: Exemplary Variant Junction Sequence 28
HHRKRR

SEQ ID NO: 98 - protein, 6 aa, synthetic construct
  REGION 1..6: note = Synthetic: Exemplary Variant Junction Sequence 29
HHRRKR

SEQ ID NO: 99 - protein, 6 aa, synthetic construct
  REGION 1..6: note = Synthetic: Exemplary Variant Junction Sequence 30
HHRRRR

SEQ ID NO: 100 - protein, 6 aa, synthetic construct
  REGION 1..6: note = Synthetic: Exemplary Variant Junction Sequence 31
HKRHKR

SEQ ID NO: 101 - protein, 6 aa, synthetic construct
  REGION 1..6: note = Synthetic: Exemplary Variant Junction Sequence 32
HKRHRR

SEQ ID NO: 102 - protein, 6 aa, synthetic construct
  REGION 1..6: note = Synthetic: Exemplary Variant Junction Sequence 33
HKRKKR

SEQ ID NO: 103 - protein, 6 aa, synthetic construct
  REGION 1..6: note = Synthetic: Exemplary Variant Junction Sequence 34
HKRKRR

SEQ ID NO: 104 - protein, 6 aa, synthetic construct
  REGION 1..6: note = Synthetic: Exemplary Variant Junction Sequence 35
HKRRKR

SEQ ID NO: 105 - protein, 6 aa, synthetic construct
  REGION 1..6: note = Synthetic: Exemplary Variant Junction Sequence 36
HKRRRR

SEQ ID NO: 106 - protein, 6 aa, synthetic construct
  REGION 1..6: note = Synthetic: Exemplary Variant Junction Sequence 37
HRRHKR

SEQ ID NO: 107 - protein, 6 aa, synthetic construct
  REGION 1..6: note = Synthetic: Exemplary Variant Junction Sequence 38
HRRHRR

SEQ ID NO: 108 - protein, 6 aa, synthetic construct
  REGION 1..6: note = Synthetic: Exemplary Variant Junction Sequence 39
HRRKKR

SEQ ID NO: 109 - protein, 6 aa, synthetic construct
  REGION 1..6: note = Synthetic: Exemplary Variant Junction Sequence 40
HRRRKR

SEQ ID NO: 110 - protein, 6 aa, synthetic construct
  REGION 1..6: note = Synthetic: Exemplary Variant Junction Sequence 41
KHRHKR

SEQ ID NO: 111 - protein, 6 aa, synthetic construct
  REGION 1..6: note = Synthetic: Exemplary Variant Junction Sequence 42
KHRHRR

SEQ ID NO: 112 - protein, 6 aa, synthetic construct
  REGION 1..6: note = Synthetic: Exemplary Variant Junction Sequence 43
KHRKKR

SEQ ID NO: 113 - protein, 6 aa, synthetic construct
  REGION 1..6: note = Synthetic: Exemplary Variant Junction Sequence 44
KHRKRR

SEQ ID NO: 114 - protein, 6 aa, synthetic construct
  REGION 1..6: note = Synthetic: Exemplary Variant Junction Sequence 45
KHRRKR

SEQ ID NO: 115 - protein, 6 aa, synthetic construct
  REGION 1..6: note = Synthetic: Exemplary Variant Junction Sequence 46
KHRRRR

SEQ ID NO: 116 - protein, 6 aa, synthetic construct
  REGION 1..6: note = Synthetic: Exemplary Variant Junction Sequence 47
KKRHKR

SEQ ID NO: 117 - protein, 6 aa, synthetic construct
  REGION 1..6: note = Synthetic: Exemplary Variant Junction Sequence 48
KKRHRR

SEQ ID NO: 118 - protein, 6 aa, synthetic construct
  REGION 1..6: note = Synthetic: Exemplary Variant Junction Sequence 49
KKRKKR

SEQ ID NO: 119 - protein, 6 aa, synthetic construct
  REGION 1..6: note = Synthetic: Exemplary Variant Junction Sequence 50
KKRKRR

SEQ ID NO: 120 - protein, 6 aa, synthetic construct
  REGION 1..6: note = Synthetic: Exemplary Variant Junction Sequence 51
KKRRKR

SEQ ID NO: 121 - protein, 6 aa, synthetic construct
  REGION 1..6: note = Synthetic: Exemplary Variant Junction Sequence 52
KKRRRR

SEQ ID NO: 122 - protein, 6 aa, synthetic construct
  REGION 1..6: note = Synthetic: Exemplary Variant Junction Sequence 53
KRRHKR

SEQ ID NO: 123 - protein, 6 aa, synthetic construct
  REGION 1..6: note = Synthetic: Exemplary Variant Junction Sequence 54
KRRHRR

SEQ ID NO: 124 - protein, 6 aa, synthetic construct
  REGION 1..6: note = Synthetic: Exemplary Variant Junction Sequence 55
KRRKKR

SEQ ID NO: 125 - protein, 6 aa, synthetic construct
  REGION 1..6: note = Synthetic: Exemplary Variant Junction Sequence 56
KRRRKR

SEQ ID NO: 126 - protein, 6 aa, synthetic construct
  REGION 1..6: note = Synthetic: Exemplary Variant Junction Sequence 57
RHRHKR

SEQ ID NO: 127 - protein, 6 aa, synthetic construct
  REGION 1..6: note = Synthetic: Exemplary Variant Junction Sequence 58
RHRHRR

SEQ ID NO: 128 - protein, 6 aa, synthetic construct
  REGION 1..6: note = Synthetic: Exemplary Variant Junction Sequence 59
RHRKKR

SEQ ID NO: 129 - protein, 6 aa, synthetic construct
  REGION 1..6: note = Synthetic: Exemplary Variant Junction Sequence 60
RHRKRR

SEQ ID NO: 130 - protein, 6 aa, synthetic construct
  REGION 1..6: note = Synthetic: Exemplary Variant Junction Sequence 61
RKRHKR

SEQ ID NO: 131 - protein, 6 aa, synthetic construct
  REGION 1..6: note = Synthetic: Exemplary Variant Junction Sequence 62
RKRHRR

SEQ ID NO: 132 - protein, 6 aa, synthetic construct
  REGION 1..6: note = Synthetic: Exemplary Variant Junction Sequence 63
RKRKKR

SEQ ID NO: 133 - protein, 6 aa, synthetic construct
  REGION 1..6: note = Synthetic: Exemplary Variant Junction Sequence 64
RKRKRR

SEQ ID NO: 134 - protein, 6 aa, synthetic construct
  REGION 1..6: note = Synthetic: Exemplary Variant Junction Sequence 65
RRRHKR

SEQ ID NO: 135 - protein, 6 aa, synthetic construct
  REGION 1..6: note = Synthetic: Exemplary Variant Junction Sequence 66
RRRHRR

SEQ ID NO: 136 - protein, 6 aa, synthetic construct
  REGION 1..6: note = Synthetic: Exemplary Variant Junction Sequence 67
RRRKKR

SEQ ID NO: 137 - protein, 20 aa, unidentified
  REGION 1..20: note = Wildtype _31_32
CGERGFFYTPKTRREAEDLQ

SEQ ID NO: 138 - protein, 20 aa, unidentified
  REGION 1..20: note = Wildtype _64_65
SLQPLALEGSLQKRGIVEQC

SEQ ID NO: 139 - protein, 20 aa, synthetic construct
  REGION 1..20: note = Synthetic: 1994 Groskreutz-Rat _31_32
CGERGFFYTPRTKREVEDPQ

SEQ ID NO: 140 - protein, 20 aa, synthetic construct
  REGION 1..20: note = Synthetic: 1994 Groskreutz-Rat _64_65
DLQTLALEVARQKRGIVDQC

SEQ ID NO: 141 - protein, 20 aa, synthetic construct
  REGION 1..20: note = Synthetic: 1994 Groskreutz-Human _31_32
CGERGFFYTPRTKREAEDLQ

SEQ ID NO: 142 - protein, 20 aa, synthetic construct
  REGION 1..20: note = Synthetic: 1994 Groskreutz-Human _64_65
SLQPLALEGSRQKRGIVEQC

SEQ ID NO: 143 - protein, 20 aa, synthetic construct
  REGION 1..20: note = Synthetic: 1992 Yanagita-Human _31_32
ERGFFYTPKTRRKREDLQVG

SEQ ID NO: 144 - protein, 20 aa, synthetic construct
  REGION 1..20: note = Synthetic: 1992 Yanagita-Human _64_65
SLQPLALEGSRRKRGIVEQC

SEQ ID NO: 145 - protein, 20 aa, synthetic construct
  REGION 1..20: note = Synthetic: ENDSULIN101_31_32
ERGFFYTPKTRRKREAEDLQ

SEQ ID NO: 146 - protein, 20 aa, synthetic construct
  REGION 1..20: note = Synthetic: ENDSULIN101_64_65
QPLALEGSLQRRKRGIVEQC

SEQ ID NO: 147 - protein, 90 aa, synthetic construct
  REGION 1..90: note = Synthetic: Exemplary proinsulin 1
FVNQHLCGSHLVEALYLVCGERGFFYTPKTRHKREAEDLQVGQVELGGGPGAGSLQPLALEGSLQRHKRGIVEQCCTSICSLYQLENYCN

SEQ ID NO: 148 - protein, 90 aa, synthetic construct
  REGION 1..90: note = Synthetic: Exemplary proinsulin 2
FVNQHLCGSHLVEALYLVCGERGFFYTPKTRHKREAEDLQVGQVELGGGPGAGSLQPLALEGSLQRHRRGIVEQCCTSICSLYQLENYCN

SEQ ID NO: 149 - protein, 90 aa, synthetic construct
  REGION 1..90: note = Synthetic: Exemplary proinsulin 3
FVNQHLCGSHLVEALYLVCGERGFFYTPKTRHKREAEDLQVGQVELGGGPGAGSLQPLALEGSLQRKKRGIVEQCCTSICSLYQLENYCN

SEQ ID NO: 150 - protein, 90 aa, synthetic construct
  REGION 1..90: note = Synthetic: Exemplary proinsulin 4
FVNQHLCGSHLVEALYLVCGERGFFYTPKTRHKREAEDLQVGQVELGGGPGAGSLQPLALEGSLQRKRRGIVEQCCTSICSLYQLENYCN

SEQ ID NO: 151 - protein, 90 aa, synthetic construct
  REGION 1..90: note = Synthetic: Exemplary proinsulin 5
FVNQHLCGSHLVEALYLVCGERGFFYTPKTRHKREAEDLQVGQVELGGGPGAGSLQPLALEGSLQRRKRGIVEQCCTSICSLYQLENYCN

SEQ ID NO: 152 - protein, 90 aa, synthetic construct
  REGION 1..90: note = Synthetic: Exemplary proinsulin 6
FVNQHLCGSHLVEALYLVCGERGFFYTPKTRHKREAEDLQVGQVELGGGPGAGSLQPLALEGSLQRRRRGIVEQCCTSICSLYQLENYCN

SEQ ID NO: 153 - protein, 90 aa, synthetic construct
  REGION 1..90: note = Synthetic: Exemplary proinsulin 7
FVNQHLCGSHLVEALYLVCGERGFFYTPKTRHRREAEDLQVGQVELGGGPGAGSLQPLALEGSLQRHKRGIVEQCCTSICSLYQLENYCN

SEQ ID NO: 154 - protein, 90 aa, synthetic construct
  REGION 1..90: note = Synthetic: Exemplary proinsulin 8
FVNQHLCGSHLVEALYLVCGERGFFYTPKTRHRREAEDLQVGQVELGGGPGAGSLQPLALEGSLQRHRRGIVEQCCTSICSLYQLENYCN

SEQ ID NO: 155 - protein, 90 aa, synthetic construct
  REGION 1..90: note = Synthetic: Exemplary proinsulin 9
FVNQHLCGSHLVEALYLVCGERGFFYTPKTRHRREAEDLQVGQVELGGGPGAGSLQPLALEGSLQRKKRGIVEQCCTSICSLYQLENYCN

SEQ ID NO: 156 - protein, 90 aa, synthetic construct
  REGION 1..90: note = Synthetic: Exemplary proinsulin 10
FVNQHLCGSHLVEALYLVCGERGFFYTPKTRHRREAEDLQVGQVELGGGPGAGSLQPLALEGSLQRKRRGIVEQCCTSICSLYQLENYCN

SEQ ID NO: 157 - protein, 90 aa, synthetic construct
  REGION 1..90: note = Synthetic: Exemplary proinsulin 11
FVNQHLCGSHLVEALYLVCGERGFFYTPKTRHRREAEDLQVGQVELGGGPGAGSLQPLALEGSLQRRKRGIVEQCCTSICSLYQLENYCN

SEQ ID NO: 158 - protein, 90 aa, synthetic construct
  REGION 1..90: note = Synthetic: Exemplary proinsulin 12
FVNQHLCGSHLVEALYLVCGERGFFYTPKTRHRREAEDLQVGQVELGGGPGAGSLQPLALEGSLQRRRRGIVEQCCTSICSLYQLENYCN

SEQ ID NO: 159 - protein, 90 aa, synthetic construct
  REGION 1..90: note = Synthetic: Exemplary proinsulin 13
FVNQHLCGSHLVEALYLVCGERGFFYTPKTRKKREAEDLQVGQVELGGGPGAGSLQPLALEGSLQRHKRGIVEQCCTSICSLYQLENYCN

SEQ ID NO: 160 - protein, 90 aa, synthetic construct
  REGION 1..90: note = Synthetic: Exemplary proinsulin 14
FVNQHLCGSHLVEALYLVCGERGFFYTPKTRKKREAEDLQVGQVELGGGPGAGSLQPLALEGSLQRHRRGIVEQCCTSICSLYQLENYCN

SEQ ID NO: 161 - protein, 90 aa, synthetic construct
  REGION 1..90: note = Synthetic: Exemplary proinsulin 15
FVNQHLCGSHLVEALYLVCGERGFFYTPKTRKKREAEDLQVGQVELGGGPGAGSLQPLALEGSLQRKKRGIVEQCCTSICSLYQLENYCN

SEQ ID NO: 162 - protein, 90 aa, synthetic construct
  REGION 1..90: note = Synthetic: Exemplary proinsulin 16
FVNQHLCGSHLVEALYLVCGERGFFYTPKTRKKREAEDLQVGQVELGGGPGAGSLQPLALEGSLQRKRRGIVEQCCTSICSLYQLENYCN

SEQ ID NO: 163 - protein, 90 aa, synthetic construct
  REGION 1..90: note = Synthetic: Exemplary proinsulin 17
FVNQHLCGSHLVEALYLVCGERGFFYTPKTRKKREAEDLQVGQVELGGGPGAGSLQPLALEGSLQRRKRGIVEQCCTSICSLYQLENYCN

SEQ ID NO: 164 - protein, 90 aa, synthetic construct
  REGION 1..90: note = Synthetic: Exemplary proinsulin 18
FVNQHLCGSHLVEALYLVCGERGFFYTPKTRKKREAEDLQVGQVELGGGPGAGSLQPLALEGSLQRRRRGIVEQCCTSICSLYQLENYCN

SEQ ID NO: 165 - protein, 90 aa, synthetic construct
  REGION 1..90: note = Synthetic: Exemplary proinsulin 19
FVNQHLCGSHLVEALYLVCGERGFFYTPKTRKRREAEDLQVGQVELGGGPGAGSLQPLALEGSLQRHKRGIVEQCCTSICSLYQLENYCN

SEQ ID NO: 166 - protein, 90 aa, synthetic construct
  REGION 1..90: note = Synthetic: Exemplary proinsulin 20
FVNQHLCGSHLVEALYLVCGERGFFYTPKTRKRREAEDLQVGQVELGGGPGAGSLQPLALEGSLQRHRRGIVEQCCTSICSLYQLENYCN

SEQ ID NO: 167 - protein, 90 aa, synthetic construct
  REGION 1..90: note = Synthetic: Exemplary proinsulin 21
FVNQHLCGSHLVEALYLVCGERGFFYTPKTRKRREAEDLQVGQVELGGGPGAGSLQPLALEGSLQRKKRGIVEQCCTSICSLYQLENYCN

SEQ ID NO: 168 - protein, 90 aa, synthetic construct
  REGION 1..90: note = Synthetic: Exemplary proinsulin 22
FVNQHLCGSHLVEALYLVCGERGFFYTPKTRKRREAEDLQVGQVELGGGPGAGSLQPLALEGSLQRKRRGIVEQCCTSICSLYQLENYCN

SEQ ID NO: 169 - protein, 90 aa, synthetic construct
  REGION 1..90: note = Synthetic: Exemplary proinsulin 23
FVNQHLCGSHLVEALYLVCGERGFFYTPKTRKRREAEDLQVGQVELGGGPGAGSLQPLALEGSLQRRKRGIVEQCCTSICSLYQLENYCN

SEQ ID NO: 170 - protein, 90 aa, synthetic construct
  REGION 1..90: note = Synthetic: Exemplary proinsulin 24
FVNQHLCGSHLVEALYLVCGERGFFYTPKTRKRREAEDLQVGQVELGGGPGAGSLQPLALEGSLQRRRRGIVEQCCTSICSLYQLENYCN

SEQ ID NO: 171 - protein, 90 aa, synthetic construct
  REGION 1..90: note = Synthetic: Exemplary proinsulin 25
FVNQHLCGSHLVEALYLVCGERGFFYTPKTRRKREAEDLQVGQVELGGGPGAGSLQPLALEGSLQRHKRGIVEQCCTSICSLYQLENYCN

SEQ ID NO: 172 - protein, 90 aa, synthetic construct
  REGION 1..90: note = Synthetic: Exemplary proinsulin 26
FVNQHLCGSHLVEALYLVCGERGFFYTPKTRRKREAEDLQVGQVELGGGPGAGSLQPLALEGSLQRHRRGIVEQCCTSICSLYQLENYCN

SEQ ID NO: 173 - protein, 90 aa, synthetic construct
  REGION 1..90: note = Synthetic: Exemplary proinsulin 27
FVNQHLCGSHLVEALYLVCGERGFFYTPKTRRKREAEDLQVGQVELGGGPGAGSLQPLALEGSLQRKKRGIVEQCCTSICSLYQLENYCN

SEQ ID NO: 174 - protein, 90 aa, synthetic construct
  REGION 1..90: note = Synthetic: Exemplary proinsulin 28
FVNQHLCGSHLVEALYLVCGERGFFYTPKTRRKREAEDLQVGQVELGGGPGAGSLQPLALEGSLQRKRRGIVEQCCTSICSLYQLENYCN

SEQ ID NO: 175 - protein, 90 aa, synthetic construct
  REGION 1..90: note = Synthetic: Exemplary proinsulin 29 ENDSULIN101-Human sequence
FVNQHLCGSHLVEALYLVCGERGFFYTPKTRRKREAEDLQVGQVELGGGPGAGSLQPLALEGSLQRRKRGIVEQCCTSICSLYQLENYCN

SEQ ID NO: 176 - protein, 90 aa, synthetic construct
  REGION 1..90: note = Synthetic: Exemplary proinsulin 30
FVNQHLCGSHLVEALYLVCGERGFFYTPKTRRKREAEDLQVGQVELGGGPGAGSLQPLALEGSLQRRRRGIVEQCCTSICSLYQLENYCN

SEQ ID NO: 177 - protein, 90 aa, synthetic construct
  REGION 1..90: note = Synthetic: Exemplary proinsulin 31
FVNQHLCGSHLVEALYLVCGERGFFYTPKTRRRREAEDLQVGQVELGGGPGAGSLQPLALEGSLQRHKRGIVEQCCTSICSLYQLENYCN

SEQ ID NO: 178 - protein, 90 aa, synthetic construct
  REGION 1..90: note = Synthetic: Exemplary proinsulin 32
FVNQHLCGSHLVEALYLVCGERGFFYTPKTRRRREAEDLQVGQVELGGGPGAGSLQPLALEGSLQRHRRGIVEQCCTSICSLYQLENYCN

SEQ ID NO: 179 - protein, 90 aa, synthetic construct
  REGION 1..90: note = Synthetic: Exemplary proinsulin 33
FVNQHLCGSHLVEALYLVCGERGFFYTPKTRRRREAEDLQVGQVELGGGPGAGSLQPLALEGSLQRKKRGIVEQCCTSICSLYQLENYCN

SEQ ID NO: 180 - protein, 90 aa, synthetic construct
  REGION 1..90: note = Synthetic: Exemplary proinsulin 34
FVNQHLCGSHLVEALYLVCGERGFFYTPKTRRRREAEDLQVGQVELGGGPGAGSLQPLALEGSLQRKRRGIVEQCCTSICSLYQLENYCN

SEQ ID NO: 181 - protein, 90 aa, synthetic construct
  REGION 1..90: note = Synthetic: Exemplary proinsulin 35
FVNQHLCGSHLVEALYLVCGERGFFYTPKTRRRREAEDLQVGQVELGGGPGAGSLQPLALEGSLQRRKRGIVEQCCTSICSLYQLENYCN

SEQ ID NO: 182 - protein, 90 aa, synthetic construct
  REGION 1..90: note = Synthetic: Exemplary proinsulin 36
FVNQHLCGSHLVEALYLVCGERGFFYTPKTRRRREAEDLQVGQVELGGGPGAGSLQPLALEGSLQRRRRGIVEQCCTSICSLYQLENYCN

SEQ ID NO: 183 - DNA, 2343 bp, synthetic construct
  misc_feature 1..2343: note = Synthetic: 1994 Groskreutz-Rat from ITR to ITR
  source 1..2343: mol_type = other DNA
cagctgcgcgctcgctcgctcactgaggccgcccgggcaaagcccgggcgtcgggcgacctttggtcgcccggcctcagtgagcgagcgagcgcgcagagagggagtggggttcctgctattcccatatgattctagagctcgtcgacccgcccccttcaccaagatctttttgatggcagagttcagtttaccgggtcacattgtacctgggaagattcaaggatttatggaaaaagtcaacaacaggagtcagagcagccggaaaagcatggactctgtacttaggactgcgctttgagcaatggcacagcaagctttaaccctgtttgcagtcagcacacaaactgtggttcaaagctccactttatctcttcttgtggaattcagatatcagatcagtttaaaccttgcggccgccagttctcacgtggtggccacgtgcttgggcactctagtgctcaaatgggagacaaagagattaagctcttatgtaaaatttgctgttttacataactttaatgaatggacaaagtcttgtgcatgggggtgggggtggggttagaggggaacagctccagatggcaaacatacgcaagggatttagtcaaacaactttttggcaaagatggtatgattttgtaatggggtaggaaccaatgaaatgcgaggtaagtatggttaataatctacagttattggttaaagaagtatattagagcgagtctttctgcacacagatcaccttcctatcaaccccactagcctctggcaaaggtaccagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggctctgtggatgagattcctgcctctgctggccctgctggtgctgtgggaacctaagcctgcccaggccttcgtgaagcagcacctgtgtggaccccacctggtggaagccctgtacctcgtgtgtggcgagagaggcttcttctacacccctaggaccaagagagaggtggaagatccccaggtgccccagctggaactgggcggaggacctgaagctggcgacctgcagacactggccctggaagtggccagacagaaaaggggcatcgtggaccagtgctgcaccagcatctgcagcctgtatcagctggaaaactactgcaactgaacgcagcctgcaggcagcgtcgagtaacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctatcgattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaagttatggattataaacattcaaaataatattttgacattatgataattctgaataaaagaacaaaaaccatggtataggtaaggaatataaaacatggcttttaccttagaaaaaacaattctaaaattcatatgattctagaactagtggggtaccgaattcagatcgagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcg

SEQ ID NO: 184 - DNA, 2343 bp, synthetic construct
  misc_feature 1..2343: note = Synthetic: 1994 Groskreutz-Human from ITR to ITR
  source 1..2343: mol_type = other DNA
cagctgcgcgctcgctcgctcactgaggccgcccgggcaaagcccgggcgtcgggcgacctttggtcgcccggcctcagtgagcgagcgagcgcgcagagagggagtggggttcctgctattcccatatgattctagagctcgtcgacccgcccccttcaccaagatctttttgatggcagagttcagtttaccgggtcacattgtacctgggaagattcaaggatttatggaaaaagtcaacaacaggagtcagagcagccggaaaagcatggactctgtacttaggactgcgctttgagcaatggcacagcaagctttaaccctgtttgcagtcagcacacaaactgtggttcaaagctccactttatctcttcttgtggaattcagatatcagatcagtttaaaccttgcggccgccagttctcacgtggtggccacgtgcttgggcactctagtgctcaaatgggagacaaagagattaagctcttatgtaaaatttgctgttttacataactttaatgaatggacaaagtcttgtgcatgggggtgggggtggggttagaggggaacagctccagatggcaaacatacgcaagggatttagtcaaacaactttttggcaaagatggtatgattttgtaatggggtaggaaccaatgaaatgcgaggtaagtatggttaataatctacagttattggttaaagaagtatattagagcgagtctttctgcacacagatcaccttcctatcaaccccactagcctctggcaaaggtaccagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcccggcagaagcgtggcattgtggaacaatgctgtaccagcatctgctccctctaccagctggagaactactgcaactagacgcagcctgcaggcagcgtcgagtaacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctatcgattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaagttatggattataaacattcaaaataatattttgacattatgataattctgaataaaagaacaaaaaccatggtataggtaaggaatataaaacatggcttttaccttagaaaaaacaattctaaaattcatatgattctagaactagtggggtaccgaattcagatcgagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcg

SEQ ID NO: 185 - DNA, 2343 bp, synthetic construct
  misc_feature 1..2343: note = Synthetic: 1992 Yanagita-Human from ITR to ITR
  source 1..2343: mol_type = other DNA
cagctgcgcgctcgctcgctcactgaggccgcccgggcaaagcccgggcgtcgggcgacctttggtcgcccggcctcagtgagcgagcgagcgcgcagagagggagtggggttcctgctattcccatatgattctagagctcgtcgacccgcccccttcaccaagatctttttgatggcagagttcagtttaccgggtcacattgtacctgggaagattcaaggatttatggaaaaagtcaacaacaggagtcagagcagccggaaaagcatggactctgtacttaggactgcgctttgagcaatggcacagcaagctttaaccctgtttgcagtcagcacacaaactgtggttcaaagctccactttatctcttcttgtggaattcagatatcagatcagtttaaaccttgcggccgccagttctcacgtggtggccacgtgcttgggcactctagtgctcaaatgggagacaaagagattaagctcttatgtaaaatttgctgttttacataactttaatgaatggacaaagtcttgtgcatgggggtgggggtggggttagaggggaacagctccagatggcaaacatacgcaagggatttagtcaaacaactttttggcaaagatggtatgattttgtaatggggtaggaaccaatgaaatgcgaggtaagtatggttaataatctacagttattggttaaagaagtatattagagcgagtctttctgcacacagatcaccttcctatcaaccccactagcctctggcaaaggtaccagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccatggccctgtggatgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaagacccgccggaagagagaggacctgcaggtggggcaggtggagctgggcgggggccctggtgcaggcagcctgcagcccttggccctggaggggtcccggcggaagcgtggcattgtggaacaatgctgtaccagcatctgctccctctaccagctggagaactactgcaactagacgcagcctgcaggcagcgtcgagtaacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctatcgattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaagttatggattataaacattcaaaataatattttgacattatgataattctgaataaaagaacaaaaaccatggtataggtaaggaatataaaacatggcttttaccttagaaaaaacaattctaaaattcatatgattctagaactagtggggtaccgaattcagatcgagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcg

SEQ ID NO: 186 - DNA, 2343 bp, synthetic construct
  misc_feature 1..2343: note = Synthetic: Negative Control from ITR to ITR
  source 1..2343: mol_type = other DNA
cagctgcgcgctcgctcgctcactgaggccgcccgggcaaagcccgggcgtcgggcgacctttggtcgcccggcctcagtgagcgagcgagcgcgcagagagggagtggggttcctgctattcccatatgattctagagctcgtcgacccgcccccttcaccaagatctttttgatggcagagttcagtttaccgggtcacattgtacctgggaagattcaaggatttatggaaaaagtcaacaacaggagtcagagcagccggaaaagcatggactctgtacttaggactgcgctttgagcaatggcacagcaagctttaaccctgtttgcagtcagcacacaaactgtggttcaaagctccactttatctcttcttgtggaattcagatatcagatcagtttaaaccttgcggccgccagttctcacgtggtggccacgtgcttgggcactctagtgctcaaatgggagacaaagagattaagctcttatgtaaaatttgctgttttacataactttaatgaatggacaaagtcttgtgcatgggggtgggggtggggttagaggggaacagctccagatggcaaacatacgcaagggatttagtcaaacaactttttggcaaagatggtatgattttgtaatggggtaggaaccaatgaaatgcgaggtaagtatggttaataatctacagttattggttaaagaagtatattagagcgagtctttctgcacacagatcaccttcctatcaaccccactagcctctggcaaaggtaccagcgcagaggcttggggcagccgagcggcagccaggccccggcccgggcctcggttccagaagggagaggagcccgccaaggcgcgcaagagagcgggctgcctcgcagtccgagccggagagggagcgcgagccgcgccggccccggacggcctccgaaaccttggccctgtggttgcgcctcctgcccctgctggcgctgctggccctctggggacctgacccagccgcagcctttgtgaaccaacacctgtgcggctcacacctggtggaagctctctacctagtgtgcggggaacgaggcttcttctacacacccaggaccaagcgggaggcagaggacctgcaggtggggcaggtggagctgggcgggggccctggtgcaggcagcctgcagcccttggccctggaggggtcccggcagaagcgtggcattgtggaacaatgctgtaccagcatctgctccctctaccagctggagaactactgcaactagacgcagcctgcaggcagcgtcgagtaacatcacatttaaaagcatctcaggtaactatattttgaattttttaaaaaagtaactataatagttattattaaaatagcaaagattgaccatttccaagagccatatagaccagcaccgaccactattctaaactatttatgtatgtaaatattagcttttaaaattctcaaaatagttgctgagttgggaaccactattatttctatcgattcagcagccgtaagtctaggacaggcttaaattgttttcactggtgtaaattgcagaaagatgatctaagtaatttggcatttattttaataggtttgaaaaacacatgccattttacaaataagacttatatttgtccttttgtttttcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaagttatggattataaacattcaaaataatattttgacattatgataattctgaataaaagaacaaaaaccatggtataggtaaggaatataaaacatggcttttaccttagaaaaaacaattctaaaattcatatgattctagaactagtggggtaccgaattcagatcgagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcg

SEQ ID NO: 187 - protein, 5 aa, synthetic construct
  REGION 1..5: note = Synthetic: Exemplary Variant Junction Sequence 23
RRKRR

SEQ ID NO: 188 - protein, 5 aa, synthetic construct
  REGION 1..5: note = Synthetic: Exemplary Variant Junction Sequence 24
RRRRR

SEQ ID NO: 189 - protein, 6 aa, synthetic construct
  REGION 1..6: note = Synthetic: Exemplary Variant Junction Sequence 68
HRRKRR

SEQ ID NO: 190 - protein, 6 aa, synthetic construct
  REGION 1..6: note = Synthetic: Exemplary Variant Junction Sequence 69
HRRRRR

SEQ ID NO: 191 - protein, 6 aa, synthetic construct
  REGION 1..6: note = Synthetic: Exemplary Variant Junction Sequence 70
KRRKRR

SEQ ID NO: 192 - protein, 6 aa, synthetic construct
  REGION 1..6: note = Synthetic: Exemplary Variant Junction Sequence 71
KRRRRR

SEQ ID NO: 193 - protein, 6 aa, synthetic construct
  REGION 1..6: note = Synthetic: Exemplary Variant Junction Sequence 72
RHRRKR

SEQ ID NO: 194 - protein, 6 aa, synthetic construct
  REGION 1..6: note = Synthetic: Exemplary Variant Junction Sequence 73
RHRRRR

SEQ ID NO: 195 - protein, 6 aa, synthetic construct
  REGION 1..6: note = Synthetic: Exemplary Variant Junction Sequence 74
RKRRKR

SEQ ID NO: 196 - protein, 6 aa, synthetic construct
  REGION 1..6: note = Synthetic: Exemplary Variant Junction Sequence 75
RKRRRR

SEQ ID NO: 197 - protein, 6 aa, synthetic construct
  REGION 1..6: note = Synthetic: Exemplary Variant Junction Sequence 76
RRRKRR

SEQ ID NO: 198 - protein, 6 aa, synthetic construct
  REGION 1..6: note = Synthetic: Exemplary Variant Junction Sequence 77
RRRRKR

SEQ ID NO: 199 - protein, 6 aa, synthetic construct
  REGION 1..6: note = Synthetic: Exemplary Variant Junction Sequence 78
RRRRRR

SEQ ID NO: 200 - protein, 86 aa, Homo sapiens
  REGION 1..86: note = misc_feature - Human Wildtype Proinsulin
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 201 - protein, 86 aa, unidentified
  REGION 1..86: note = Canine Wildtype Proinsulin
FVNQHLCGSHLVEALYLVCGERGFFYTPKARREVEDLQVRDVELAGAPGEGGLQPLALEGALQKRGIVEQCCTSICSLYQLENYCN

SEQ ID NO: 202 - protein, 86 aa, unidentified
  REGION 1..86: note = Feline Wildtype Proinsulin
FVNQHLCGSHLVEALYLVCGERGFFYTPKARREAEDLQGKDAELGEAPGAGGLQPSALEAPLQKRGIVEQCCASVCSLYQLEHYCN

SEQ ID NO: 203 - protein, 30 aa, synthetic construct
  REGION 1..30: note = Synthetic: Exemplary Modified Human B-Chain
FVNQHLCGSDLVEALYLVCGERGFFYTPKT

SEQ ID NO: 204 - protein, 4 aa, synthetic construct
  REGION 1..4: note = Synthetic
  REGION 1..4: note = misc_feature - junction between the B-chain and C-peptide
KTRR

SEQ ID NO: 205 - protein, 4 aa, synthetic construct
  REGION 1..4: note = Synthetic
  REGION 1..4: note = misc_feature - junction between the B-chain and C-peptide
RTKR

SEQ ID NO: 206 - protein, 4 aa, synthetic construct
  REGION 1..4: note = Synthetic
  REGION 1..4: note = misc_feature - junction between the C-peptide and A-chain
LQKR

SEQ ID NO: 207 - protein, 4 aa, synthetic construct
  REGION 1..4: note = Synthetic
  REGION 1..4: note = misc_feature - junction between the C-peptide and A-chain
RQKR